(19) United States
(12) Patent Application Publication
(10) Pub. No.: US 2015/0099643 A1
(43) Pub. Date: Apr. 9, 2015

(54) BLOOD-BASED GENE EXPRESSION SIGNATURES IN LUNG CANCER

(71) Applicants: RHEINISCHE FRIEDRICH-WILHELMS-UNIVERSITAT BONN, Bonn (DE); UNIVERSITAT ZU KOLN, Koln (DE)

(72) Inventors: Andrea HOFMANN, Bonn (DE); Joachim L. SCHULTZE, Konigswinter (DE); Jurgen WOLF, Koln (DE); Andrea STARATSCHEK-JOX, Koln (DE); Thomas ZANDER, Bonn (DE)

(21) Appl. No.: 14/328,365

(22) Filed: Jul. 10, 2014

Related U.S. Application Data
(63) Continuation of application No. 14/115,562.

(30) Foreign Application Priority Data
May 2, 2011 (EP) 11164471.2

(51) Int. Cl. C12Q 1/68

(52) U.S. Cl. CPC C12Q 1/6886; C12Q 2600/158; C12Q 2600/16
USPC 506/2; 506/7; 506/9; 506/16; 506/17

(57) ABSTRACT

The invention pertains to a method for diagnosing or detecting lung cancer in human subjects based on ribonucleic acid (RNA) expression, in particular based on RNA from blood. The invention discloses 361 genes which are differentially expressed in blood from lung cancer patients and discloses that at least 4 of the mRNAs must be determined in order to have an AUC of at least 0.8.

The invention pertains to a method for diagnosing or detecting lung cancer in human subjects based on ribonucleic acid (RNA), in particular based on RNA from blood.

### INTRODUCTION

Lung cancer is the leading cause of cancer-related death worldwide. Prognosis has remained poor with a disastrous two-year survival rate of only about 15% due to diagnosis of the disease in late, i.e. incurable stages in the majority of patients (Jemal A, Siegel R, Ward E, et al. Cancer statistics, 2008. CA Cancer J Clin 2008; 58: 71-96) and still disappointing therapeutic regimens in advanced disease (Sandler A, Gray R, Perry M C, et al., Paclitaxel-carboplatin alone or with bevacizumab for non-small-cell lung cancer. N Engl J Med 2006; 355: 2542-50). Thus far, the only way to detect lung cancer is by means of imaging technologies detecting morphological changes in the lung in combination with biopsy specimens taken for histological examination. However, these screening approaches are not easily applied to secondary prevention of lung cancer in an asymptomatic population (Henschke C I, Yankelevitz D F, Libby D M, Pasmantier M W, Smith J P, Miettinen O S. Survival of patients with stage I lung cancer detected on CT screening. N Engl J Med 2006; 355: 1763-71). Thus, there is an urgent need in the art to establish reliable tools for the identification of lung cancer patients at early stages of the disease, e.g. prior to the development of clinical symptoms.

### BRIEF DESCRIPTION OF THE INVENTION

The inventors have surprisingly found means to satisfy this need. Accordingly, the present invention provides methods and kits for diagnosing, detecting, and screening for lung cancer. Particularly, the invention provides for preparing RNA expression profiles of patient blood samples, the RNA expression profiles being indicative of the presence or absence of lung cancer. The invention further provides for evaluating the patient RNA expression profiles for the presence or absence of one or more RNA expression signatures that are indicative of lung cancer.

In one aspect, the invention provides a method for preparing RNA expression profiles that are indicative of the presence or absence of lung cancer. The RNA expression profiles are prepared from patient blood samples. The number of transcripts in the RNA expression profile may be selected so as to offer a convenient and cost effective means for screening samples for the presence or absence of lung cancer with high sensitivity and high specificity. Generally, the RNA expression profile includes the expression level or “abundance” of from 4 to about 3000 transcripts. In certain embodiments, the expression profile includes the RNA levels of 2500 transcripts or less, 2000 transcripts or less, 1500 transcripts or less, 1000 transcripts or less, 500 transcripts or less, 250 transcripts or less, 100 transcripts of less, or 50 transcripts or less.

In such embodiments, the profile may contain the abundance or expression level of at least 4 RNAs that are indicative of the presence or absence of lung cancer, and specifically, as selected from table 3, optionally together with at least 1 RNA from the RNAs listed in table 3b, or may contain the expression level of at least 9, at least 10, at least 13 or at least 29 RNAs selected from tables 3 and/or 3b. Where larger profiles are desired, the profile may contain the expression level or abundance of at least about 60, at least 100, at least 157, or 161 RNAs that are indicative of the presence or absence of lung cancer, and such RNAs may be selected from tables 3 and/or 3b. The identities and/or combinations of genes and/or transcripts that make up or are included in expression profiles are disclosed in tables 3, 3b, and 5 to 8.

Such RNA expression profiles in accordance with this aspect may be evaluated for the presence or absence of an RNA expression signature indicative of lung cancer. Generally, the sequential addition of transcripts from tables 3 and/or 3b to the expression profile provides for higher sensitivity and/or specificity for the detection of lung cancer. For example, the area under the ROC curve (AUC) may be at least at least 0.8, or at least 0.82, or at least 0.85 or at least 0.9. The AUC is a quantitative parameter for the clinical utility (specificity and sensitivity) of the detection method described herein. An AUC of 1.0 refers to a sensitivity and specificity of 100%.

In contrast to traditional molecular diagnostic methods, there is no single molecule or gene that suffices as a biomarlcer to determine disease status reliably. Rather, only a combination of RNAs from tables 3 and/or 3b can achieve an adequate clinical utility for diagnosing or detecting lung cancer in human subjects. This combination is achieved through machine learning algorithms, for example, support vector machines, Nearest-Neighbors, Decision Trees, Logistic Regression, Articifial Neural Networks, or Rule-based schemes. Different combinations of RNAs have specific properties, such as a specific area under the curve (AUC), or specific combinations of sensitivity and specificity.

In a second aspect, the invention provides a method for detecting, diagnosing, or screening for lung cancer. In this aspect, the method comprises preparing an RNA expression profile by measuring the abundance of at least 4, at least 9, at least 10, or at least 13, or at least 29 RNAs in a patient blood sample, where the abundance of such RNAs are indicative of the presence or absence of lung cancer. The RNAs may be selected from the RNAs listed in table 3 and/or table 3b, and exemplary sets of such RNAs are disclosed in tables 3 to 8. In one embodiment of the invention, the RNAs may be selected from the RNAs listed in table 3b or be chosen from the RNAs listed in table 3b in addition to RNAs listed in table 3. The method further comprises evaluating the profile for the presence or absence of an RNA expression signature indicative of lung cancer, to thereby conclude whether the patient has or does not have lung cancer. The method generally provides a sensitivity for the detection of lung cancer of at least about 70%, while providing a specificity of at least about 70%.

In various embodiments, the method comprises determining the abundance of at least 4 RNAs, at least 60 RNAs, at least 100 RNAs, at least 157, or of at least 161 RNAs chosen from the RNAs listed in tables 3 and/or 3b, and as exemplified in tables 3, 3b, 4 to 8, and classifying the sample as being indicative of lung cancer, or not being indicative of lung cancer.

In other aspects, the invention provides kits and custom arrays for preparing the gene expression profiles, and for determining the presence or absence of lung cancer.

### DETAILED DESCRIPTION OF THE INVENTION

The invention provides methods and kits for screening, diagnosing, and detecting lung cancer in human patients (subjects). “Lung cancer” (LC) refers to both non-small cell lung cancer (NSCLC) and small cell lung cancer (SCLC).

Lung cancer is composed of two major different histologies: non-small cell lung cancer and small cell lung cancer. Within the group of non-small cell lung cancer, three main histological subgroups are described: adenocarcinoma, squamous cell carcinoma and large cell carcinoma. All subtypes are described in the WHO classification of 2004 (Travis et al., 2004). Lung cancer clinically presents in different stages that are defined by UICC (Goldstraw, Peter; Crowley, John; Chansky, Kari; Giroux, Dorothy J; Groome, Patti A; Rami-Porta, Ramon; Postmus, Pieter E; Rusch, Valerie; Sobin, Leslie M D; on behalf of the International Association for the Study of Lung Cancer International staging committee and participating institutions (2007); The IASLC Lung Cancer Staging Project: proposals for the revision of the TNM stage groupings in the forthcoming (seventh) edition of the TNM Classification of Malignant Tumours. Journal of Thoracic Oncology 2(8): 706-714).

A synonym for a patient with lung cancer is “LC-case” or simply “case.”

As disclosed herein, the present invention provides methods and kits for screening patient samples for those that are positive for LC, e.g., in the absence of surgery or any other diagnostic procedure.

The invention relates to the determination of the abundance of RNAs to detect a lung cancer in a human subject, wherein the determination of the abundance is based on RNA obtained (or isolated) from whole blood of the subject. The term “whole blood” refers to a sample of blood taken from a human individual for which no separation of particular fractions of the blood is performed. In particular, no separation of a certain type of blood cell or of blood cells in general needs to be performed, since the whole blood sample is used in the present invention. This allows for easier handling and shipping of the blood samples compared to methods in which the blood sample is separated into different fractions and a particular fraction is then used for RNA isolation.

In various aspects, the invention involves preparing an RNA expression profile from a patient sample. The method may comprise isolating RNA from whole blood, and detecting the abundance or relative abundance of selected transcripts. The “RNAs” may be defined by reference to an expressed gene, or by reference to a transcript, or by reference to a particular oligonucleotide probe for detecting the RNA (or cDNA derived therefrom), each of which is listed in table 3 for 161 RNAs and in table 3b for 200 RNAs that are indicative of the presence or absence of lung cancer.

The number of transcripts in the RNA expression profile may be selected so as to offer a convenient and cost effective means for screening samples for the presence or absence of lung cancer with high sensitivity and high specificity: For example, the RNA expression profile may include the expression level or “abundance” of from 4 to about 3000 transcripts. In certain embodiments, the expression profile includes the RNA levels of 2500 transcripts or less, 2000 transcripts or less, 1500 transcripts or less, 1000 transcripts or less, 500 transcripts or less, 250 transcripts or less, 200 transcripts of less, 100 transcripts of less, or 50 transcripts or less. Such profiles may be prepared, for example, using custom microarrays or multiplex gene expression assays as described in detail herein.

Such RNA expression profiles in accordance with this aspect may be evaluated for the presence or absence of an RNA expression signature indicative of lung cancer. Generally, the sequential addition of transcripts from table 3 or from table 3b to the expression profile provides for higher sensitivity and/or specificity for the detection of lung cancer, as indicated by the AUC. A clinical utility is reached if the AUC is at least 0.8.

The inventors have surprisingly found that an AUC of 0.8 is reached if and only if at least 4 RNAs are measured that are chosen from the RNAs listed in table 3. In other words, measuring 4 RNAs is necessary and sufficient for the detection of lung cancer in a human subject based on RNA from a blood sample obtained from said subject by measuring the abundance of at least 4 RNAs in the sample, that are chosen from the RNAs listed in table 3 or in table 3b, and concluding based on the measured abundance whether the subject has lung cancer or not. An analysis of 1, 2 or 3 RNAs chosen from the RNAs listed in table 3 or table 3b, however, does not allow for this detection.

For example, the area under the ROC curve (AUC) may be at least 0.8, or at least 0.82, or at least 0.85 or at least 0.9. The AUC is a quantitative parameter for the clinical utility (specificity and sensitivity) of the detection method described herein. An AUC of 1.0 refers to a sensitivity and specificity of 100%.

In such embodiments, the profile may contain the expression level of at least 4 RNAs that are indicative of the presence or absence of lung cancer, and specifically, as selected from table 3 and/or table 3b, or may contain the expression level of at least 9, 10, 13 or 29 RNAs selected from table 3. Where larger profiles are desired, the profile may contain the expression level or abundance of at least 60, 100, 200, 500, 1000 RNAs, or 2000 RNAs that are indicative of the presence or absence of lung cancer, and such RNAs may be (at least in part) selected from tables 3 and/or 3b. Such RNAs may be defined by gene, or by transcript ID, or by probe ID.

The identities of genes and/or transcripts that make up, or are included in exemplary expression profiles are disclosed in tables 3, 3b, and 5. As shown herein, profiles selected from the RNAs of tables 3 and/or 3b support the detection of lung cancer with high sensitivity and high specificity. Exemplary selections of RNAs for the RNA expression profile are shown in tables 6 to 8.

Thus, in various embodiments, the abundance of at least 4, at least 9, at least 29, at least 60, at least 100, at least 157, or at least 161 distinct RNAs are measured, in order to arrive at a reliable diagnosis of lung cancer. The set of RNAs may comprise, consist essentially of, or consist of, a set or subset of RNAs exemplified in any one of tables 3, 3b and 5 to 8. The term “consists essentially of” in this context allows for the expression level of additional transcripts to be determined that are not differentially expressed in lung cancer subjects, and which may therefore be used as positive or negative expression level controls or for normalization of expression levels between samples.

Such RNA expression profiles may be evaluated for the presence or absence of an RNA expression signature indicative of lung cancer. Generally, the sequential addition of transcripts from tables 3 and/or 3b to the expression profile provides for higher sensitivity and/or specificity and stability (i.e. independence from the sample analyzed) for the detection of lung cancer. For example, the sensitivity and specificity of the methods provided herein may be equivalent to an area under the ROC curve (AUC) of at least at least 0.8, or at least 0.82, at least 0.85, or of at least 0.9.

The present invention provides an in-vitro diagnostic test system (IVD) that is trained (as described further below) for the detection of lung cancer. For example, in order to determine whether a patient has lung cancer, reference RNA abundance values for lung cancer positive and negative samples are determined. The RNAs can be quantitatively measured on an adequate set of training samples comprising cases and controls, and with adequate clinical information on carcinoma status, applying adequate quality control measures, and on an adequate set of test samples, for which the detection is yet to be made. With such quantitative values for the RNAs and the clinical data for the training samples, a classifier can be trained and applied to the test samples to calculate the probability of the presence or non-presence of the lung carcinoma. Therefore, in one embodiment of the present method, a sample can be classified as being from a patient with lung cancer or from a healthy individual without the necessity to run a reference sample of known origin (i.e. from a lung cancer patient or a healthy individual) at the same time.

Various classification schemes are known for classifying samples between two or more classes or groups, and these include, without limitation: Naïve Bayes, Support Vector Machines, Nearest Neighbors, Decision Trees, Logistic Regression, Articifial Neural Networks, and Rule-based schemes. In addition, the predictions from multiple models can be combined to generate an overall prediction. Thus, a classification algorithm or “class predictor” may be constructed to classify samples. The process for preparing a suitable class predictor is reviewed in R. Simon, Diagnostic and prognostic prediction using gene expression profiles in high-dimensional microarray data, British Journal of Cancer (2003) 89, 1599-1604, which review is hereby incorporated by reference.

In this context, the invention teaches an in-vitro diagnostic test system (IVD) that is trained in the detection of a lung cancer referred to above, comprising at least 4 RNAs, which can be quantitatively measured on an adequate set of training samples comprising cases and controls, with adequate clinical information on carcinoma status, applying adequate quality control measures, and on an adequate set of test samples, for which the detection yet has to be made. Given the quantitative values for the RNAs and the clinical data for the training samples, a classifier can be trained and applied to the test samples to calculate the probability of the presence or absence of the lung carcinoma.

The present invention provides methods for detecting, diagnosing, or screening for lung cancer in a human subject with a high sensitivity and specificity. Specifically, the sensitivity of the methods provided herein is equivalent to an area under the ROC curve (AUC) of at least at least 0.8, or at least 0.82, at least 0.85, of or at least 0.9.

Without wishing to be bound by any particular theory, the above finding may be due to the fact that an organism such as a human systemically reacts to the development of a lung tumor by altering the expression levels of genes in different pathways. Although the change in expression (abundance) might be small for each gene in a particular signature, measuring a set of at least 4 genes, preferably even larger numbers such as 9, 10, 13, 29, 100, 157, 161 or even more RNAs, for example at least 5, at least 8, at least 120, at least 160 RNAs at the same time allows for the detection of lung cancer in a human with high sensitivity and high specificity.

In this context, an RNA obtained from a subject's whole blood sample, i.e. an RNA biomarker, is an RNA molecule with a particular base sequence whose presence within a blood sample from a human subject can be quantitatively measured. The measurement can be based on a part of the RNA molecule, namely a part of the RNA molecule that has a certain base sequence, which allows for its detection and thereby allows for the measurement of its abundance in a sample. The measurement can be by methods known in the art, for example analysis on a solid phase device (for example on arrays or beads), or in solution (for example, by RT-PCR). Probes for the particular RNAs can either be bought commercially, or designed based on the respective RNA sequence.

In the method of the invention, the abundance of several RNA molecules (e.g. mRNA or pre-spliced RNA, intron-lariat RNA, micro RNA, small nuclear RNA, or fragments thereof) is determined in a relative or an absolute manner, wherein an absolute measurement of RNA abundance is preferred. The RNA abundance is, if applicable, compared with that of other individuals, or with multivariate quantitative thresholds, or evaluated as part of a classification algorithm with respect to training and normalization data.

The determination of the abundance of the RNAs described herein is performed from blood samples using quantitative methods. In particular, RNA is isolated from a blood sample obtained from a human subject that is to undergo lung cancer testing, e.g. a smoker. Although the examples described herein use microarray-based methods, the invention is not limited thereto. For example, RNA abundance can be measured by in situ hybridization, amplification assays such as the polymerase chain reaction (PCR), sequencing, or microarray-based methods. Other methods that can be used include polymerase-based assays, such as RT-PCR (e.g., TAQMAN), hybridization-based assays, such as DNA microarray analysis, as well as direct mRNA capture with branched DNA (QUANTIGENE) or HYBRID CAPTURE (DIGENE). Direct transcript sequencing by Next Generation Sequencing methods represents another possibility.

In certain embodiments, the invention employs a microarray. A “micoroarray” includes a specific set of probes, such as oligonucleotides and/or cDNAs (e.g., expressed sequence tags, “ESTs”) corresponding in whole or in part, and/or continuously or discontinuously, to regions of RNAs that can be extracted from a blood sample of a human subject. The probes are bound to a solid support. The support may be selected from beads (magnetic, paramagnetic, etc.), glass slides, and silicon wafers. The probes can correspond in sequence to the RNAs of the invention such that hybridization between the RNA from the subject sample (or cDNA derived therefrom) and the probe occurs. In the microarray embodiments, the sample RNA can optionally be amplified before hybridization to the microarray. Prior to hybridization, the sample RNA is fluorescently labeled. Upon hybridization to the array and excitation at the appropriate wavelength, fluorescence emission is quantified. Fluorescence emission for each particular RNA is directly correlated with the amount of the particular RNA in the sample. The signal can be detected and together with its location on the support can be used to determine which probe hybridized with RNA from the subject's whole blood sample.

Accordingly, in certain aspects, the invention is directed to a kit or microarray for detecting the level of expression or abundance of RNAs in the subject's blood sample, where this “profile” allows for the conclusion of whether the subject has lung cancer or not (at a level of accuracy described herein). In another aspect, the invention relates to a probe set that allows for the detection of the RNAs associated with LC. If these particular RNAs are present in a sample, they (or corresponding cDNA) will hybridize with their respective probe (i.e, a complementary nucleic acid sequence), which will yield a detectable signal. Probes are designed to minimize cross reactivity and false positives.

Thus, the invention in certain aspects provides a microarray, which generally comprises a solid support and a set of oligonucleotide probes. The set of probes generally contains from 4 to about 3,000 probes, including at least 4 probes deduced from tables 3, 3b, or 5 to 8. In certain embodiments, the set contains 2000 probes or less, or 1000 probes or less, 500 probes or less, 200 probes or less, or 100 probes or less.

The conclusion whether the subject has lung cancer or not is preferably reached on the basis of a classification algorithm, which can be developed using e.g. a random forest method, a support vector machine (SVM), a K-nearest neighbor method (K-NN), such as a 3-nearest neighbor method (3-NN), a linear discrimination analysis (LDA), or a prediction analysis for microarrays (PAM), as known in the art.

Preferably, F-statistics (ANOVA) is used to identify specific difference of the abundance of the at least 4 RNAs in healthy individuals versus the abundance of the at least 4 RNAs in individuals with lung cancer.

“Sensitivity” (S⁺ or true positive fraction (TPF)) refers to the count of positive test results among all true positive disease states divided by the count of all true positive disease states. “Specificity” (S⁻ or true negative fraction (TNF)) refers to the count of negative test results among all true negative disease states divided by the count of all true negative disease states. “Correct Classification Rate” (CCR or true fraction (TF)) refers to the sum of the count of positive test results among all true positive disease states and count of negative test results among all true negative disease states divided by all the sum of all cases. The measures S⁺, S⁻, and CCR address the question: To what degree does the test reflect the true disease state?

“Positive Predictive Value” (PV⁺ or PPV) refers to the count of true positive disease states among all positive test results dived by the count of all positive test results. “Negative Predictive Value” (PV⁻ or NPV) refers to the count of true negative disease states among all negative test results dived by the count of all negative test results. The predictive values address the question: How likely is the disease given the test results?

The preferred RNA molecules that can be used in combinations described herein for diagnosing and detecting lung cancer in a subject according to the invention can be found in tables 3 and/or 3b. The inventors have shown that the selection of at least 4 or more RNAs of the markers listed in tables 3 and/or 3b can be used to diagnose or detect lung cancer in a subject using a blood sample from that subject. The RNA molecules that can be used for detecting, screening and diagnosing lung cancer are selected from the RNAs provided in tables 3, 3b or 5.

Specifically, the method of the invention comprises at least the following steps: measuring the abundance of at least 4 RNAs (preferably 9 RNAs or 10 RNAs) in the sample, that are chosen from the RNAs listed in table 3 and/or table 3b, and concluding, based on the measured abundance, whether the subject has lung cancer or not. Measuring the abundance of RNAs may comprise isolating RNA from blood samples as described, and hybridizing the RNA or cDNA prepared therefrom to a microarray. Alternatively, other methods for determining RNA levels may be employed.

Examples for sets of 4 RNAs that are measured together, i.e. sequentially or preferably simultaneously, are shown in tables 6, 7, and 8. The sets of at least 4 RNAs of tables 6, 7 and 8 are defined by a common threshold of AUC>=0.8.

In a preferred embodiment of the invention as mentioned herein, the abundance of at least 4 RNAs (preferably 9, 10, or 13 RNAs) in the sample is measured, wherein the at least 4 RNAs are chosen from the RNAs listed in table 3 and/or table 3b. Examples for sets of 4 RNAs that can be measured together, i.e. sequentially or preferably simultaneously, to detect lung cancer in a human subject are shown in tables 6, 7, and 8. The sets of RNAs of table 6 (4, 9, 10, 13, 29 RNAs) are defined by a common threshold of AUC>=0.8.

Similarly, the abundance of at least 9 RNAs (preferably up to 29 RNAs), of at least 30 RNAs (preferably up to 59 RNAs), of at least 60 RNAs (preferably up to 99 RNAs), of at least 100 RNAs (preferably up to 160 RNAs), of at least 16 RNAs that are chosen from the RNAs listed in table 3 and/or table 3b can be measured in the method of the invention.

An example for a set of 161 RNAs of which the abundance can be measured in the method of the invention is listed in table 3. An example for a set of 200 RNAs of which the abundance can be measured in the method of the invention is listed in table 3b.

When the wording “at least a number of RNAs” is used, this refers to a minimum number of RNAs that are measured. It is possible to use up to 10,000 or 20,000 genes in the invention, a fraction of which can be RNAs listed in table 3 and/or in table 3b. In preferred embodiments of the invention, abundance of up to 5.000, 2.500, 2.000, 1,000, 500, 250, 100, 80, 70, 60, 50, 40, 30, 20, 10, 5, 4, 3, 2, or 1 RNA of randomly chosen RNAs that are not listed in tables 3 or 3b is measured in addition to RNAs of table 3 (or subsets thereof).

In a preferred embodiment, only RNAs that are mentioned in table 3 are measured. In another preferred embodiment, only RNAs that are mentioned in table 3b are measured. In another preferred embodiment, only RNAs are measured that are mentioned in table 3 together with RNAs that are mentioned in table 3b are measured (“combination signatures”).

The expression profile or abundance of RNA markers for lung cancer, for example the at least 4 RNAs described above, (or more RNAs as disclosed above and herein), is determined preferably by measuring the quantity of the transcribed RNA of the marker gene. This quantity of the mRNA of the marker gene can be determined for example through chip technology (microarray), (RT-) PCR (for example also on fixated material), Northern hybridization, dot-blotting, sequencing, or in situ hybridization.

The microarray technology, which is most preferred, allows for the simultaneous measurement of RNA abundance of up to many thousand RNAs and is therefore an important tool for determining differential expression (or differences in RNA abundance), in particular between two biological samples or groups of biological samples. In order to apply the microarray technology, the RNAs of the sample need to be amplified and labeled and the hybridization and detection procedure can be performed as known to a person of skill in the art.

As will be understood by those of ordinary skill in the art, the analysis can also be performed through single reverse transcriptase-PCR, competitive PCR, real time PCR, differential display RT-PCR, Northern blot analysis, sequencing, and other related methods. In general, the larger the number of markers is that are to be measured, the more preferred is the use of the microarray technology. However, multiplex PCR, for example, real time multiplex PCR is known in the art and is amenable for use with the present invention, in order to detect the presence of 2 or more genes or RNAs simultaneously.

The RNA whose abundance is measured in the method of the invention can be mRNA, cDNA, unspliced RNA, or its fragments. Measurements can be performed using the complementary DNA (cDNA) or complementary RNA (cRNA), which is produced on the basis of the RNA to be analyzed, e.g. using microarrays. A great number of different arrays as well as their manufacture are known to a person of skill in the art and are described for example in the U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,331; 5,554,501; 5,561,071; 5,571,639; 5,593,839; 5,599,695; 5,624,711; 5,658,734; and 5,700,637.

Preferably the decision whether the subject has lung cancer comprises the step of training a classification algorithm on an adequate training set of cases and controls and applying it to RNA abundance data that was experimentally determined based on the blood sample from the human subject to be diagnosed. The classification method can be a random forest method, a support vector machine (SVM), or a K-nearest neighbor method (K-NN), such as 3-NN.

For the development of a model that allows for the classification for a given set of biomarkers, such as RNAs, methods generally known to a person of skill in the art are sufficient, i.e. new algorithms need not be developed.

The major steps of such a model are:

1) condensation of the raw measurement data (for example combining probes of a microarray to probeset data, and/or normalizing measurement data against common controls);
2) training and applying a classifier (i.e. a mathematical model that generalizes properties of the different classes (carcinoma vs. healthy individual) from the training data and applies them to the test data resulting in a classification for each test sample.

For example, the raw data from microarray hybridizations can first be condensed with FARMS as shown by Hochreiter (2006, Bioinformatics 22(8): 943-9). Alternative methods for condensation such as Robust Multi-Array Analysis (RMA, GC-RMA, see Irizarry et al (2003). Exploration, Normalization, and Summaries of High Density Oligonucleotide Array Probe Level Data. Biostatistics. 4, 249-264.) can be used. Similar to condensation, classification of the test data set through a support-vector-machine or other classification algorithms is known to a person of skill in the art, like for example classification and regression trees, penalized logistic regression, sparse linear discriminant analysis, Fisher linear discriminant analysis, K-nearest neighbors, shrunken centroids, and artificial neural networks (see Wladimir Wapnik: The Nature of Statistical Learning Theory, Springer Verlag, New York, N.Y., USA, 1995; Berhard Schölkopf, Alex Smola: Learning with Kernels: Support Vector Machines, Regularization, Optimization, and Beyond, MIT Press, Cambridge, Mass., 2002; S. Kotsiantis, Supervised Machine Learning: A Review of Classification Techniques, Informatica Journal 31 (2007) 249-268).

The key component of these classifier training and classification techniques is the choice of RNA biomarkers that are used as input to the classification algorithm.

In a further aspect, the invention refers to the use of a method as described above and herein for the detection of lung cancer in a human subject, based on RNA from a blood sample.

In a further aspect, the invention also refers to the use of a microarray for the detection of lung cancer in a human subject based on RNA from a blood sample. According to the invention, such a use can comprise measuring the abundance of at least 4 RNAs (or more, as described above and herein) that are listed in tables 3 and/or 3b. Accordingly, the microarray comprises at least 4 probes for measuring the abundance of the at least 5 RNAs. Commercially available microarrays, such as from Illumina or Affymetrix, may be used.

In another embodiment, the abundance of the at least 4 RNAs is measured by multiplex RT-PCR. In a further embodiment, the RT-PCR includes real time detection, e.g., with fluorescent probes such as Molecular beacons or TaqMan® probes.

In a preferred embodiment, the microarray comprises probes for measuring only RNAs that are listed in table 3 or in table 3b (or subsets thereof).

In yet a further aspect, the invention also refers to a kit for the detection of lung cancer in a human subject based on RNA obtained from a blood sample. Such a kit comprises a means for measuring the abundance of at least 4 RNAs that are chosen from the RNAs listed in tables 3 and/or 3b. The means for measuring expression can be probes that allow for the detection of RNA in the sample or primers that allow for the amplification of RNA in the sample. Ways to devise probes and primers for such a kit are known to a person of skill in the art.

Further, the invention refers to the use of a kit as described above and herein for the detection of lung cancer in a human subject based on RNA from a blood sample comprising means for measuring the abundance of at least 4 RNAs that are chosen from the RNAs listed in tables 3 and/or 3b. Such a use may comprise the following steps: contacting at least one component of the kit with RNA from a blood sample from a human subject, measuring the abundance of at least 4 RNAs (or more as described above and herein) that are chosen from the RNAs listed in tables 3 and/or 3b using the means for measuring the abundance of at least 4 RNAs, and concluding, based on the measured abundance, whether the subject has lung cancer.

In yet a further aspect, the invention also refers to a method for preparing an RNA expression profile that is indicative of the presence or absence of lung cancer, comprising: isolating RNA from a whole blood sample, and determining the level or abundance of from 4 to about 3000 RNAs, including at least 4 RNAs selected from tables 3 and/or 3b.

Preferably, the expression profile contains the level or abundance of 161 RNAs or less, 157 or less, of 150 RNAs or less, or of 100 RNAs or less. Further, it is preferred that at least 10 RNAs, at least 30 RNAs, at least 100 RNAs are listed in tables 3, 3b or tables 6, 7, or 8.

In yet a further aspect, the invention also refers to a microarray, comprising a solid support and a set of oligonucleotide probes, the set containing from 4 to about 3,000 probes, and including at least 4 probes selected from tables 3, 3b or 5. Preferably, the set contains 161 probes or less (such as e.g. 157 probes, or less), or 200 probes or less (such as e.g. 187 probes, or less). At least 10 probes can be those listed in table 3, table 3b, or table 6. At least 30 probes can be those listed in table 3, table 3b, or table 5. In another embodiment, at least 100 probes are listed in table 3, table 3b, or table 6.

Features of the invention that were described herein in combination with a method, a microarray, a kit, or a use also refer, if applicable, to all other aspects of the invention.

### FIGURES

FIG. 1 shows the experimental design of the study that lead to aspects of the invention. In a training group (A) feature selection and classifier detection was performed. Read-out was performed using the area under the curve (AUC) for each cut-off of F-statistics and each algorithm. (B) A classifier was applied to the validation groups (PG2, PG3). (C, E) A random permutation was performed (n=1000) (D) to test specificity.

FIG. 2 shows the mean area under the receiver operator curve (AUC) plotted against the cut-off of the F-statistics for feature selection for all three algorithms (SVM, LDA, PAM) obtained in the 10-fold cross-validation in PG1. An SVM leads to the highest single mean AUC and is therefore preferred. (SVM: dark; LDA: lower curve shown, green; PAM: upper curve shown, red).

FIG. 3 shows the classifier for prevalent lung cancer. The AUC and the 95% confidence interval are given (A). Box plots visualizing the SVM probabilities for cases and controls in the validation group (box=25-75 percentile; whisker=10-90 percentile; dot=5-95 percentile) (B). The box plot comprises permuted AUCs. The real AUC (real data) is depicted in red (C).

FIG. 4 shows the mean area under the receiver operator curve (AUC) is plotted against the cut-off of the F-statistics for feature selection in PG1 and PG2. Both prevalent groups (PG1 & PG2) were pooled and a 10-fold cross-validation (9:1 dataset splitting) was performed. The cut-off for the F-statistics for feature selection was continuously increased from 0.00001-0.1. For each cross-validation, the AUC for the receiver operator curve was calculated. The mean+/−2 standard deviation is plotted. For better visualization, a line is drawn at 0.5 (AUC obtained by chance). (A) A detailed view in the area of the maximum AUC is shown. An additional box blot visualizes the AUC obtained by random lists at the respective cut-off of the F-statistics (box=25-75 percentile; whisker=10-90 percentile; dot=all outliers). (B) The overlap in the genes extracted for the respective classifier is depicted (C).

FIG. 5 shows the receiver operator curve of unmatched cases with prevalent lung cancer (n=22) and controls (n=21) (PG3). The false discovery rate (1—specificity) is plotted against the true discovery rate (sensitivity). The diagonal with an area under the curve of 0.5 is plotted for better visualization. The AUC was calculated with 0.727. At the maximum Youden index, the sensitivity was 0.90 and the specificity 0.64. SVM probabilities for cases and controls were significantly different (Student's T test p=0.0047). The AUC and the 95% confidence interval are given.

FIG. 6: All samples were ordered by the present call rate. The present call rate (darker; dark blue) for each sample and the respective deviation of the mean from the overall mean (lighter; dark red) is plotted. Those samples declared of low quality (present call rate=light blue; light; deviation from the mean=light red; dark) are highlighted.

### TABLES

Table 1: Clinical and Epidemiological Characteristics of Cases with Lung Cancer and Respective Controls.

Clinical and epidemiological characteristics of cases and controls in the three groups with prevalent lung cancer (PG1, PG2, PG3) are given.

Table 2: Detailed Clinical and Epidemiological Characteristics of Cases with Lung Cancer and Controls Recruited for the Study

Clinical and epidemiological characteristics of all patients are given. Age, gender and pack years of smoked cigarettes are given. For lung cancer cases, the histopathological diagnosis is displayed. Finally, co-morbidity was documented using the ICD-10 code. NA=Not analyzed.

### Table 3: Annotation of Features Used for the Classifiers

The feature list used in the classifier is demonstrated: The 161 features selected in the ten-fold cross-validation in PG1 and applied to PG2. In the column up vs. down 1=upregulation in lung cancer patients; −1=downregulation in lung cancer patients. The RNAs listed in this table can be used for the detection of lung cancer according to the invention. Each RNA is identified by SEQ ID NO, gene symbol, gene name, refseq ID, and entrez ID, as used elsewhere in the application.

### Table 3b:

Table 3b shows a list of 200 RNAs that are differentially expressed in several human subjects with lung cancer in comparison to subjects without lung cancer. According to the invention, the abundance of RNAs, preferably of at least 4 RNAs, from the list of RNAs shown in table 3 is measured, optionally together with a number of RNAs taken from the list of RNAs of table 3b. It is also possible to measure the abundance of at least 4 (preferably of 9, 10, 13, or 29) RNAs of table 3b alone. Examples of signatures consisting of RNAs from table 3 together with RNAs from table 3b (“combination signatures”) as well as from table 3b alone are given below in tables 7 and 8, respectively. Each of the ranked RNAs is identified by SEQ ID NO, gene symbol, gene name, refseq ID, and ranking score.

### Table 4: Annotation of Features Differentially Expressed Most Robustly

31 transcripts demonstrating a stable differential expression over all data-set splitting between cases and controls.

### Table 5: Annotation of Features Differentially Expressed

1000 features with differential expression between lung cancer (NSCLC and SCLC) and controls.

### Table 6:

Table 6 shows exemplary sets of RNAs from table 3 whose abundance in a blood sample from a human individual can be determined according to the invention to detect lung cancer in the individual. Each list shows a set of RNAs (defined by probe set and gene name) with an area under the curve (AUC) of at least 0.8. The AUC is a quantitative parameter for the clinical utility (specificity and sensitivity) of the detection method described herein. An AUC of 1.0 refers to a sensitivity and specificity of 100%.

### Table 7:

Table 7 show exemplary sets of RNAs from table 3 and table 3b (“combination signatures”) whose abundance in a blood sample from a human individual can be determined according to the invention to detect lung cancer in the individual. Each list shows a set of RNAs (defined by probe set and gene name) with an area under the curve (AUC) of at least 0.8. The AUC is a quantitative parameter for the clinical utility (specificity and sensitivity) of the detection method described herein. An AUC of 1.0 refers to a sensitivity and specificity of 100%.

### Table 8:

Table 8 show exemplary sets of RNAs from table 3b whose abundance in a blood sample from a human individual can be determined according to the invention to detect lung cancer in the individual. Each list shows a set of RNAs (defined by probe set and gene name) with an area under the curve (AUC) of at least 0.8. The AUC is a quantitative parameter for the clinical utility (specificity and sensitivity) of the detection method described herein. An AUC of 1.0 refers to a sensitivity and specificity of 100%.

TABLE 1

| | | |
|---|---|---|
| test group | validation group | validation group |
| PG1 | PG2 | PG3 |

| | case | control | case | control | case | control |
|---|---|---|---|---|---|---|
| total number | 42 | 42 | 13 | 11 | 22 | 21 |
| female | 13 | 14 | 6 | 5 | 7 | 8 |
| male | 29 | 28 | 7 | 6 | 15 | 13 |
| NSCLC | 35 | NA | 11 | NA | 17 | NA |
| SCLC | 7 | NA | 2 | NA | 5 | NA |
| median age | 62 | 61 | 62 | 61 | 63 | 57 |
| (years) | | | | | | |
| stage = I | 5 | NA | 4 | NA | 3 | NA |
| stage>1 | 37 | NA | 9 | NA | 19 | NA |

TABLE 2
Columns from left to right are: Sample ID; groups; case/control [case = 1; control = 0]; age (years);
gender [male = M; female = F]; histology [Adenoc. = AD; squamous cell c. = SQ; large cell c. = LC;
small cell c. = SC; small cell lung c. = SCLC; not applicable = NA]; stage [UICC stage 1-4; not
applicable = NA]; latency to clinical manifest lung cancer [months; not applicable = NA]; smoking
status [current/ever = CU = 2; ex-smoker = EX = 1; never-smoker = NEV]; packyears; comorbidity
ICD-10

| Sample | | | | | | | | Smoking | Pack | |
|---|---|---|---|---|---|---|---|---|---|---|
| ID | Group | Class | Age | Gender | Histology | Stage | Latency | Status | years | Comorbidity |
| 389918 | PG1 | 0 | 63 | F | NA | NA | NA | 1 | 31 | M53.26, M54.4, I10, E03.9 |
| 389920 | PG1 | 0 | 64 | F | NA | NA | NA | 2 | 27 | D22.7, I89.0, I10, E66, M35.3, E78.0, Z88.0, J45 |
| 389922 | PG1 | 0 | 73 | M | NA | NA | NA | 1 | 87 | M48.06, M54.4, E78.0, M06.0, J45, Z95.0, I49.9 |
| 389956 | PG1 | 0 | 70 | M | NA | NA | NA | 1 | 54 | T84.0, Z96.6, I25.1, I10, E87.6 |
| 389958 | PG1 | 0 | 45 | M | NA | NA | NA | 1 | 35 | C43.4, E04.9, E11.9, I10 |
| 389960 | PG1 | 0 | 62 | F | NA | NA | NA | 1 | 28 | |
| 389962 | PG1 | 0 | 71 | F | NA | NA | NA | 1 | 30 | |
| 389964 | PG1 | 0 | 57 | F | NA | NA | NA | 1 | 26 | M48.06, M54.4 |
| 389966 | PG1 | 0 | 58 | M | NA | NA | NA | 1 | 28 | C44.9, E78.0, I10, I20, M10 |
| 389968 | PG1 | 0 | 73 | M | NA | NA | NA | 1 | 34 | M48.06, M54.4, I73.9, E10, I49.9, E79.0, E78, I10 |
| 389970 | PG1 | 0 | 58 | F | NA | NA | NA | 1 | 20 | |
| 389972 | PG1 | 0 | 68 | M | NA | NA | NA | 2 | 25 | C44.2, I10, L30, M51.2 |
| 389973 | PG1 | 0 | 60 | F | NA | NA | NA | 1 | 49 | C43, G43, M79.7, E03.9 |
| 389985 | PG1 | 0 | 65 | F | NA | NA | NA | 1 | 22 | M48.06, M54.4, E03.9, F32 |
| 389987 | PG1 | 0 | 63 | M | NA | NA | NA | 1 | 54 | |
| 389988 | PG1 | 0 | 59 | M | NA | NA | NA | 1 | 73 | D04.3, L57.0 |
| 389938 | PG1 | 0 | 71 | M | NA | NA | NA | 1 | 58 | L40, I10, L27.0 |
| 389940 | PG1 | 0 | 61 | M | NA | NA | NA | 2 | 62 | M48.02, M50.0+ |
| 389942 | PG1 | 0 | 71 | M | NA | NA | NA | 1 | 16 | B86, L02.0, I10, L40 |
| 389989 | PG1 | 0 | 68 | M | NA | NA | NA | 1 | 53 | C44.3, E11, E78.0, I10, I73.9 |
| 389991 | PG1 | 0 | 59 | M | NA | NA | NA | 2 | 57 | L30.1, B95.6, Z88.0 |
| 389975 | PG1 | 0 | 61 | M | NA | NA | NA | 2 | 38 | M16.1, E11, H40 |
| 389978 | PG1 | 0 | 63 | M | NA | NA | NA | 1 | 28 | C44.3, E04.0, N40 |
| 389979 | PG1 | 0 | 58 | F | NA | NA | NA | 1 | 43 | |
| 389924 | PG1 | 0 | 54 | M | NA | NA | NA | 2 | 43 | C43, E78.0, Z86.7, Z92.1 |
| 389926 | PG1 | 0 | 47 | F | NA | NA | NA | 2 | 34 | L25, L29 |
| 389910 | PG1 | 0 | 66 | M | NA | NA | NA | 1 | 14 | C44.3, I10, E78.0, E79.0, T81.4 |
| 389912 | PG1 | 0 | 54 | F | NA | NA | NA | 2 | 26 | M42, M47, M99.3 |
| 389914 | PG1 | 0 | 61 | M | NA | NA | NA | 1 | 62 | M16.1, I10, E78.0, E79.0 |
| 389929 | PG1 | 0 | 55 | M | NA | NA | NA | 1 | 33 | M16.1, K50, L89 |
| 389930 | PG1 | 0 | 60 | M | NA | NA | NA | 1 | 65 | M61.5, F32 |
| 389932 | PG1 | 0 | 57 | F | NA | NA | NA | 2 | 62 | M50.2, E11, I10, E66, I49.9, I50 |
| 389934 | PG1 | 0 | 63 | M | NA | NA | NA | 1 | 30 | M48.06, M54.4, I10, E79.0 |
| 389936 | PG1 | 0 | 53 | M | NA | NA | NA | 1 | 3 | S42.2, Z47.0 |
| 389944 | PG1 | 0 | 43 | M | NA | NA | NA | 2 | 28 | |
| 389946 | PG1 | 0 | 64 | M | NA | NA | NA | 1 | 54 | M54.4, M99.7, I49.9, N40, E78.0 |
| 389948 | PG1 | 0 | 69 | M | NA | NA | NA | 1 | 49 | L20, L29, I10, I50, D50, Z85.0 |
| 389950 | PG1 | 0 | 67 | M | NA | NA | NA | 1 | 14 | M17.1, M99.3, G95.1, N40, I35.0, I10 |
| 389952 | PG1 | 0 | 57 | M | NA | NA | NA | 1 | 32 | M30.1, I10, J45, N40 |
| 389954 | PG1 | 0 | 68 | M | NA | NA | NA | 2 | 36 | S32.0 |
| 389915 | PG1 | 0 | 58 | F | NA | NA | NA | 2 | 1 | M16, E03.9. E78.0, H33, N80, D25 |
| 389983 | PG1 | 0 | 55 | F | NA | NA | NA | 2 | 21 | M48.06, M54.4 |
| 389917 | PG1 | 1 | 65 | F | AD | Ia | 0 | 2 | 33 | I10, Z86.1, Z85.5 |
| 389919 | PG1 | 1 | 64 | F | AD | IIb | 0 | 2 | 29 | J44, E78, I70, I10 |
| 389921 | PG1 | 1 | 70 | M | AD | IIIa | 0 | 1 | 87 | K70.3, G62.1, K25.−, I10, I50, I73.9, H27.1, H62.9, J44 |
| 389955 | PG1 | 1 | 76 | M | SCLC | IIIa | 0 | 2 | 56 | |
| 389957 | PG1 | 1 | 46 | M | AD | IV | 0 | 1 | 34 | Z88.0 |
| 389959 | PG1 | 1 | 59 | F | AD | IIIa | 0 | 2 | 28 | E05.8, Y57.9, M03.6, K21.9, K43.9 |
| 389961 | PG1 | 1 | 75 | F | SCLC | IV | 0 | 2 | 39 | |
| 389963 | PG1 | 1 | 56 | F | AD | IIIa | 0 | 1 | 28 | I10, E03 |
| 389965 | PG1 | 1 | 58 | M | AD | IV | 0 | 2 | 30 | F17.2 |
| 389967 | PG1 | 1 | 75 | M | AD | IIb | 0 | 1 | 34 | I25.9, I20.9, I10, N40, E79.0 |
| 389969 | PG1 | 1 | 61 | F | SCLC | IV | 0 | 1 | 20 | |
| 389971 | PG1 | 1 | 71 | M | LC | IIIa | 0 | 1 | 26 | Z85.5, N18.82, I15.1, R53, J96.1 |
| 389974 | PG1 | 1 | 60 | F | AD | IIIa | 0 | 2 | 48 | E89.0, E51 |
| 389986 | PG1 | 1 | 62 | M | SCLC | IV | 0 | 2 | 79 | |
| 389937 | PG1 | 1 | 72 | M | AD | Ib | 0 | 1 | 60 | I10, I25 |
| 389939 | PG1 | 1 | 61 | M | SQ | IV | 0 | 2 | 60 | I73.9, I10 |
| 389941 | PG1 | 1 | 70 | M | SCLC | IIIb | 0 | 1 | 21 | I10, G52.2 |
| 389990 | PG1 | 1 | 57 | M | SQ | IIIA | 0 | 1 | 58 | |
| 389992 | PG1 | 1 | 72 | M | AD | IIIa | 0 | 2 | 50 | K25, N28.1, Z89.0 |
| 389976 | PG1 | 1 | 60 | M | SQ | Ib | 0 | 2 | 40 | M48.0, N31.9, C05.2, Z85.8, T78.4 |
| 389977 | PG1 | 1 | 62 | M | SQ | IV | 0 | 1 | 28 | I25.2, I69.3 |
| 389980 | PG1 | 1 | 59 | F | SCLC | IV | 0 | 1 | 20 | |
| 389923 | PG1 | 1 | 62 | M | SQ | IIIa | 0 | 2 | 65 | J44.2, I25.9, I25.2 |
| 389925 | PG1 | 1 | 55 | M | SCLC | IIIa | 0 | 1 | 34 | |
| 389909 | PG1 | 1 | 65 | M | AD | IIIb | 0 | 1 | 14 | I10, E89.0 |
| 389911 | PG1 | 1 | 52 | F | AD | IIIb | 0 | 2 | 27 | M41, K21.9 |
| 389913 | PG1 | 1 | 61 | M | SQ | Ia | 0 | 2 | 60 | J42, E66.9, I25.12, E14.9, I74.0, E78.2, I10, I50.12, |
| | | | | | | | | | | I73.9 |
| 389927 | PG1 | 1 | 60 | F | AD | IIb | 0 | 1 | 2 | K31.88, M10.0 |
| 389928 | PG1 | 1 | 47 | F | AD | IIIa | 0 | 2 | 35 | K22.7, C37, Z90.8, K44, T78.4 |
| 389931 | PG1 | 1 | 60 | F | SQ | IV | 0 | 2 | 59 | I10, E89.0, J44, Z90.3, H40 |
| 389933 | PG1 | 1 | 63 | M | SQ | IIa | 0 | 1 | 32 | I10, I25.19, I71.4 |
| 389935 | PG1 | 1 | 52 | M | AD | IV | 0 | 1 | 2 | |
| 389943 | PG1 | 1 | 45 | M | AD | IV | 0 | 2 | 31 | |
| 389945 | PG1 | 1 | 63 | M | AD | IIIB | 0 | 1 | 52 | |
| 389947 | PG1 | 1 | 68 | M | AD | IIb | 0 | 1 | 45 | Z86.1, Z90.3, Z90.4 |
| 389949 | PG1 | 1 | 68 | M | AD | IIIa | 0 | 1 | 14 | |
| 389951 | PG1 | 1 | 58 | M | SQ | IIIa | 0 | 2 | 35 | None |
| 389953 | PG1 | 1 | 71 | M | AD | Ia | 0 | 2 | 36 | I10, E89.0, Z85.8 |
| 389916 | PG1 | 1 | 53 | M | AD | IV | 0 | 2 | 45 | J37.0 |
| 389981 | PG1 | 1 | 57 | M | AD | IIIa | 0 | 1 | 74 | J38.0 |
| 389982 | PG1 | 1 | 67 | M | SQ | IIIb | 0 | 2 | 53 | J44 |
| 389984 | PG1 | 1 | 54 | F | AD | IIIa | 0 | 2 | 24 | I25.9, I64, I25.3, I51.3 |
| 320333 | PG2 | 0 | 71 | M | NA | NA | NA | 2 | 9 | M65, M25.4, Z96.6, N40, N39.4 |
| 320330 | PG2 | 0 | 61 | M | NA | NA | NA | 2 | 114 | C44.3, J45, Z88.0 |
| 320332 | PG2 | 0 | 62 | F | NA | NA | NA | 1 | 7 | M16.1, D17.0, Z98.1, T81.0 |
| 320361 | PG2 | 0 | 72 | M | NA | NA | NA | 1 | 26 | C43, C79.3, C61, Z95.2, I10, E78.0, K80, Z88.0, |
| 320362 | PG2 | 0 | 50 | F | NA | NA | NA | 2 | 30 | L23, L40, I10 |
| 320363 | PG2 | 0 | 67 | F | NA | NA | NA | 1 | 17 | M48.06, M54.4, M16, J45 |
| 320365 | PG2 | 0 | 54 | M | NA | NA | NA | 2 | 37 | M53.26, M96.1, I10, J42 |
| 320328 | PG2 | 0 | 53 | F | NA | NA | NA | 1 | 25 | M16.1, Z96.6, E78.0 |
| 320329 | PG2 | 0 | 58 | M | NA | NA | NA | 2 | 37 | Z01.5, I50, I10, E79.0, Z88.0, Z88.4 |
| 320339 | PG2 | 0 | 70 | F | NA | NA | NA | 1 | 28 | M48.06, M54.4, M42, I35.1, M17, I10, |
| 320331 | PG2 | 0 | 48 | M | NA | NA | NA | 2 | 29 | M42, M51.2 |
| 320319 | PG2 | 1 | 69 | M | AD | Ib | 0 | 2 | 64 | K38.9, E14, J42, J96.9, I42.0, I20.9, I10 |
| 320337 | PG2 | 1 | 63 | M | SQ | IIIa | 0 | 1 | 35 | I10, M15.9, L40.0; N18.9; K25, Z96.6 |
| 320338 | PG2 | 1 | 71 | F | SQ | IIb | 0 | 1 | 49 | 170.2, I10, I27.0, I25.2, I25.1, Z95.0, Z95.2, E11, J44 |
| 320325 | PG2 | 1 | 56 | F | AD | IIIb-IV | 0 | 1 | 42 | I11.9, J44.1, I10, E87.6 |
| 320326 | PG2 | 1 | 48 | F | AD | IV | 0 | 2 | 37 | I30.9 |
| 320323 | PG2 | 1 | 62 | F | AD | IIb | 0 | 1 | 23 | J44.8 |
| 320324 | PG2 | 1 | 67 | F | AD | I | 0 | 2 | 56 | H34.2, K25, F32.9, C08.0 |
| 320336 | PG2 | 1 | 50 | M | SQ | Ia | 0 | 2 | 27 | I20, I70.2, E78.2, I25.12, I25.22, N18, F12 |
| 320335 | PG2 | 1 | 68 | M | SCLC | IV | 0 | 2 | 44 | C79.3, E03.9, H74.8, F17.1 |
| 320320 | PG2 | 1 | 69 | M | AD | IV | 0 | 2 | 40 | I10, I69.3, J44, |
| 320334 | PG2 | 1 | 52 | F | SCLC | Ib | 0 | 2 | 32 | C56 |
| 320321 | PG2 | 1 | 73 | M | AD | IIIA | 0 | 1 | 35 | I64, I10, I71.4 |
| 320322 | PG2 | 1 | 53 | M | LC | IV | 0 | 2 | 14 | K08.9, T78.1 |
| 320385 | PG3 | 0 | 76 | M | NA | NA | NA | 1 | 39 | C44.3, I10, E78.0, L80, D33.3 |
| 320376 | PG3 | 0 | 67 | M | NA | NA | NA | 1 | 60 | B02.7, C90.0, Z94.8, I10 |
| 320377 | PG3 | 0 | 65 | M | NA | NA | NA | 2 | 11 | M48.06, M54.4, G95.1, M42 |
| 320379 | PG3 | 0 | 70 | F | NA | NA | NA | 2 | 26 | L40, E66, I10, M17, K45, E61.1 |
| 320380 | PG3 | 0 | 76 | M | NA | NA | NA | 1 | 45 | M17, E11, I10, C61 |
| 320382 | PG3 | 0 | 67 | M | NA | NA | NA | 1 | 37 | T84.5, Z96.6, M17, I63.9, G20, F32 |
| 320383 | PG3 | 0 | 46 | M | NA | NA | NA | 2 | 27 | C49.2, H66.9, H90 |
| 320369 | PG3 | 0 | 65 | F | NA | NA | NA | 1 | 49 | |
| 320370 | PG3 | 0 | 69 | M | NA | NA | NA | 1 | 105 | |
| 320371 | PG3 | 0 | 73 | M | NA | NA | NA | 1 | 53 | |
| 320372 | PG3 | 0 | 50 | F | NA | NA | NA | 2 | 33 | L50, L23.0, M81 |
| 320373 | PG3 | 0 | 51 | F | NA | NA | NA | 1 | 33 | Q82.2, Z88.1, Z88.6, E89.0 |
| 320375 | PG3 | 0 | 62 | M | NA | NA | NA | 1 | 38 | M46.4, M42, K26, I10, E78 |
| 320366 | PG3 | 0 | 68 | M | NA | NA | NA | 1 | 37 | M19.9, Z98.1, I10 |
| 320367 | PG3 | 0 | 61 | F | NA | NA | NA | 1 | 7 | M51.2, M54.4, G57.6 |
| 390036 | PG3 | 0 | 30 | F | NA | NA | NA | 2 | NA | |
| 390035 | PG3 | 0 | 45 | F | NA | NA | NA | 0 | NA | |
| 390033 | PG3 | 0 | 44 | M | NA | NA | NA | 0 | NA | |
| 390038 | PG3 | 0 | 32 | M | NA | NA | NA | 1 | NA | |
| 390034 | PG3 | 0 | 37 | M | NA | NA | NA | 0 | NA | |
| 390037 | PG3 | 0 | 36 | F | NA | NA | NA | 1 | NA | |
| 320350 | PG3 | 1 | 55 | M | AD | IV | 0 | 2 | 41 | K80.2 |
| 320351 | PG3 | 1 | 71 | M | AD | IIIB | 0 | 2 | 34 | |
| 320352 | PG3 | 1 | 62 | M | AD | IIIB | 0 | 2 | 40 | |
| 320353 | PG3 | 1 | 61 | M | SQ | IIIA | 0 | 1 | 68 | E11, I10, E79.0 |
| 320354 | PG3 | 1 | 69 | M | SQ | Ib | 0 | 2 | 62 | M42.16, I21.9, R09.1, N20.0, I10 |
| 320355 | PG3 | 1 | 62 | M | SQ | IIIB | 0 | 1 | 10 | |
| 320318 | PG3 | 1 | 68 | F | SQ | IIIA | 0 | 2 | NA | D32, J44, M51.2 |
| 320327 | PG3 | 1 | 63 | F | AD | IV | 0 | 2 | 48 | K38.9, E02, N15.10 |
| 320340 | PG3 | 1 | 72 | M | SCLC | IV | 0 | 1 | 94 | I10, E1, N08.3, N40 |
| 320341 | PG3 | 1 | 53 | F | SCLC | IIIB | 0 | 2 | 54 | None |
| 320342 | PG3 | 1 | 72 | M | SQ | IIIB | 0 | 2 | 104 | S68.1, D35.0 |
| 320343 | PG3 | 1 | 78 | M | SQ | IIIA | 0 | 1 | 53 | |
| 320344 | PG3 | 1 | 61 | M | AD | Ia | 0 | 1 | 50 | |
| 320345 | PG3 | 1 | 52 | F | AD | IV | 0 | 2 | 30 | |
| 320346 | PG3 | 1 | 62 | F | AD | IV | 0 | 2 | 11 | |
| 320347 | PG3 | 1 | 57 | M | AD | IB | 0 | 2 | 85 | F10.2 |
| 320348 | PG3 | 1 | 67 | F | SCLC | IV | 0 | 2 | 29 | J42, E89.0, I40, J44 |
| 320349 | PG3 | 1 | 47 | M | SCLC | IV | 0 | 2 | 26 | |
| 320356 | PG3 | 1 | 77 | M | AD | IIIA | 0 | 1 | 37 | I69.3, A16.8, A17, B90.0, G45, J15, M81, M24.66, I10 |
| 320357 | PG3 | 1 | 61 | M | AD | IV | 0 | 0 | NA | Z96.6, I10 |
| 320358 | PG3 | 1 | 50 | F | SCLC | II | 0 | 2 | 31 | |
| 320359 | PG3 | 1 | 69 | M | SQ | IIIA | 0 | 2 | 39 | J44, I35.0 |

TABLE 3
RNAs for prevalent LC of all stages

| SEQ | | | | | | | Over-(1)/ |
|---|---|---|---|---|---|---|---|
| ID | | | | | | | under-(−1) |
| NO. | ID | Symbol | Gene name | Refseq | Entrez | p-Value | expression |
| 1 | 10541 | TM6SF1 | Homo sapiens transmembrane 6 superfamily | NM_023003 | 53346 | 0.000242963 | 1 |
| | | | member 1 (TM6SF1), transcript variant 1, mRNA | | | | |
| 2 | 10543 | ANKRD13A | Homo sapiens ankyrin repeat domain 13A | NM_033121 | 88455 | 9.80737E−05 | 1 |
| | | | (ANKRD13A), mRNA | | | | |
| 3 | 70022 | LCOR | Homo sapiens ligand dependent nuclear receptor | NM_032440 | 84458 | 5.86843E−05 | 1 |
| | | | corepressor (LCOR), transcript variant 1, mRNA | | | | |
| 4 | 110706 | CTBS | Homo sapiens chitobiase, di-N-acetyl-(CTBS), | NM_004388 | 1486 | 0.000125747 | 1 |
| | | | mRNA. | | | | |
| 5 | 130113 | SLC25A25 | Homo sapiens solute carrier family 25 | NM_001006641 | 114789 | 0.00055226 | −1 |
| | | | (mitochondrial carrier; phosphate carrier), member | | | | |
| | | | 25 (SLC25A25), nuclear gene encoding | | | | |
| | | | mitochondrial protein, transcript variant 2, mRNA. | | | | |
| 6 | 160132 | CREB5 | Homo sapiens cAMP responsive element binding | NM_001011666 | 9586 | 0.000670388 | 1 |
| | | | protein 5 (CREB5), transcript variant 4, mRNA. | | | | |
| 7 | 270717 | PELI2 | Homo sapiens pellino homolog 2 (Drosophila) | NM_021255 | 57161 | 0.000145125 | 1 |
| | | | (PELI2), mRNA. | | | | |
| 8 | 430382 | UBE2G1 | Homo sapiens ubiquitin-conjugating enzyme E2G | NM_003342 | 7326 | 1.32288E−05 | −1 |
| | | | 1 (UBC7 homolog, yeast) (UBE2G1), mRNA. | | | | |
| 9 | 450037 | LY9 | Homo sapiens lymphocyte antigen 9 (LY9), | NM_001033667 | 4063 | 0.00051284 | −1 |
| | | | transcript variant 2, mRNA. | | | | |
| 10 | 460608 | TNFSF13B | Homo sapiens tumor necrosis factor (ligand) | NM_006573 | 10673 | 0.000795241 | 1 |
| | | | superfamily, member 13b (TNFSF13B), transcript | | | | |
| | | | variant 1, mRNA. | | | | |
| 11 | 510450 | RCC2 | Homo sapiens regulator of chromosome | NM_018715 | 55920 | 5.45662E−05 | −1 |
| | | | condensation 2 (RCC2), transcript variant 1, | | | | |
| | | | mRNA. | | | | |
| 12 | 520332 | GALT | Homo sapiens galactose-1-phosphate | NM_000155 | 2592 | 3.80634E−05 | −1 |
| | | | uridylyltransferase (GALT), mRNA. | | | | |
| 13 | 610563 | HMGB2 | Homo sapiens high mobility group box 2 | NM_002129 | 3148 | 0.000645674 | 1 |
| | | | (HMGB2), transcript variant 1, mRNA. | | | | |
| 14 | 650164 | CYP4F3 | Homo sapiens cytochrome P450, family 4, | NM_000896 | 4051 | 0.000132547 | −1 |
| | | | subfamily F, polypeptide 3 | | | | |
| | | | (CYP4F3), transcript variant 1, mRNA. | | | | |
| 15 | 650767 | PPP2R5A | Homo sapiens protein phosphatase 2, regulatory | NM_006243 | 5525 | 0.000372093 | 1 |
| | | | subunit B′, alpha | | | | |
| | | | (PPP2R5A), transcript variant 1, mRNA. | | | | |
| 16 | 670041 | IL23A | Homo sapiens interleukin 23, alpha subunit p19 | NM_016584 | 51561 | 0.000435839 | −1 |
| | | | (IL23A), mRNA. | | | | |
| 17 | 870370 | XPO4 | Homo sapiens exportin 4 (XPO4), mRNA. | NM_022459 | 64328 | 0.000714824 | −1 |
| 18 | 940132 | FNIP1 | Homo sapiens folliculin interacting protein 1 | NM_001008738 | 96459 | 0.000398007 | 1 |
| | | | (FNIP1), transcript variant 2, mRNA. | | | | |
| 19 | 1110215 | ESYT1 | Homo sapiens extended synaptotagmin-like | NM_015292 | 23344 | 0.00030992 | −1 |
| | | | protein 1 (ESYT1), transcript variant 2, mRNA | | | | |
| 20 | 1110600 | EIF4E3 | Homo sapiens eukaryotic translation initiation | NM_173359 | 317649 | 2.05726E−05 | 1 |
| | | | factor 4E family | | | | |
| | | | member 3 (EIF4E3), transcript variant 2, mRNA. | | | | |
| 21 | 1240603 | ITGAX | Homo sapiens integrin, alpha X (complement | NM_000887 | 3687 | 0.000164472 | 1 |
| | | | component 3 receptor 4 | | | | |
| | | | subunit) (ITGAX), mRNA. | | | | |
| 22 | 1400762 | CPA3 | Homo sapiens carboxypeptidase A3 (mast cell) | NM_001870 | 1359 | 7.19574E−05 | 1 |
| | | | (CPA3), mRNA. | | | | |
| 23 | 1430292 | SLC11A1 | Homo sapiens solute carrier family 11 (proton- | NM_000578 | 6556 | 0.000556791 | 1 |
| | | | coupled divalent | | | | |
| | | | metal ion transporters), member 1 (SLC11A1), | | | | |
| | | | mRNA. | | | | |
| 24 | 1440601 | CDK5RAP1 | Homo sapiens CDK5 regulatory subunit | NM_016082 | 51654 | 0.000651058 | −1 |
| | | | associated protein 1 | | | | |
| | | | (CDK5RAP1), transcript variant 2, mRNA. | | | | |
| 25 | 1450184 | C5orf41 | Homo sapiens chromosome 5 open reading frame | NM_153607 | 153222 | 0.000417493 | −1 |
| | | | 41 (C5orf41), transcript variant 1, mRNA. | | | | |
| 26 | 1580168 | PRSS12 | Homo sapiens protease, serine, 12 (neurotrypsin, | NM_003619 | 8492 | 0.000226126 | −1 |
| | | | motopsin) (PRSS12), mRNA. | | | | |
| 27 | 1690189 | HSPA8 | Homo sapiens heat shock 70 kDa protein 8 | NM_006597 | 3312 | 0.000720224 | −1 |
| | | | (HSPA8), transcript variant 1, mRNA. | | | | |
| 28 | 1770131 | TSPAN2 | Homo sapiens tetraspanin 2 (TSPAN2), mRNA | NM_005725 | 10100 | 7.36749E−05 | 1 |
| 29 | 1780348 | IMP3 | Homo sapiens IMP3, U3 small nucleolar | NM_018285 | 55272 | 0.000632967 | −1 |
| | | | ribonucleoprotein, homolog (yeast) (IMP3), | | | | |
| | | | mRNA. | | | | |
| 30 | 1820255 | NA | Homo sapiens cDNA FLJ46626 fis, clone | AK_128481 | NA | 5.49124E−06 | −1 |
| | | | TRACH2001612. | | | | |
| 31 | 1820598 | ICAM2 | Homo sapiens intercellular adhesion molecule 2 | NM_000873 | 3384 | 0.000398072 | −1 |
| | | | (ICAM2), transcript variant 5, mRNA. | | | | |
| 32 | 2000390 | CDK14 | Homo sapiens cyclin-dependent kinase 14 | NM_012395 | 5218 | 0.000382729 | 1 |
| | | | (CDK14), mRNA. | | | | |
| 33 | 2030482 | RPS6KA5 | Homo sapiens ribosomal protein S6 kinase, | NM_004755 | 9252 | 0.000159455 | 1 |
| | | | 90 kDa, polypeptide 5 (RPS6KA5), transcript | | | | |
| | | | variant 1, mRNA. | | | | |
| 34 | 2060279 | PAK2 | Homo sapiens p21 protein (Cdc42/Rac)-activated | NM_002577 | 5062 | 6.07456E−05 | −1 |
| | | | kinase 2 (PAK2), mRNA. | | | | |
| 35 | 2070152 | CMTM6 | Homo sapiens CKLF-like MARVEL | NM_017801 | 54918 | 0.000549761 | 1 |
| | | | transmembrane domain containing 6 (CMTM6), | | | | |
| | | | mRNA | | | | |
| 36 | 2100035 | STK17B | Homo sapiens serine/threonine kinase 17b | NM_004226 | 9262 | 0.00022726 | 1 |
| | | | (STK17B), mRNA. | | | | |
| 37 | 2100427 | RUNX1 | Homo sapiens runt-related transcription factor 1 | NM_001001890 | 861 | 0.000684129 | −1 |
| | | | (RUNX1), transcript variant 2, mRNA. | | | | |
| 38 | 2260239 | MXD1 | Homo sapiens MAX dimerization protein 1 | NM_002357 | 4084 | 0.000517481 | 1 |
| | | | (MXD1), transcript variant 1, mRNA | | | | |
| 39 | 2370524 | TNFAIP6 | Homo sapiens tumor necrosis factor, alpha- | NM_007115 | 7130 | 1.41338E−07 | 1 |
| | | | induced protein 6 (TNFAIP6), mRNA. | | | | |
| 40 | 2450064 | ZFP91 | Homo sapiens zinc finger protein 91 homolog | NM_053023 | 80829 | 0.000223977 | −1 |
| | | | (mouse) (ZFP91), transcript variant 1, mRNA. | | | | |
| 41 | 2450497 | NA | FB22G11 Fetal brain, Stratagene Homo sapiens | T03068.1 | NA | 0.000709867 | −1 |
| | | | cDNA clone FB22G11 3-end, mRNA sequence | | | | |
| 42 | 2510639 | UBE2Z | Homo sapiens ubiquitin-conjugating enzyme E2Z | NM_023079 | 65264 | 0.000247902 | −1 |
| | | | (UBE2Z), mRNA. | | | | |
| 43 | 2570703 | C17orf97 | Homo sapiens chromosome 17 open reading | NM_001013672 | 400566 | 5.19791E−06 | −1 |
| | | | frame 97 (C17or197), mRNA. | | | | |
| 44 | 2630154 | GABARAPL1 | Homo sapiens GABA(A) receptor-associated | NM_031412 | 23710 | 0.000412487 | 1 |
| | | | protein like 1 (GABARAPL1), mRNA. | | | | |
| 45 | 2630451 | HIST2H2BE | Homo sapiens histone cluster 2, H2be | NM_003528 | 8349 | 0.000520526 | 1 |
| | | | (HIST2H2BE), mRNA. | | | | |
| 46 | 2630484 | ATP10B | Homo sapiens ATPase, class V, type 10B | NM_025153 | 23120 | 0.000154203 | 1 |
| | | | (ATP10B), mRNA. | | | | |
| 47 | 2650075 | AP1S1 | Homo sapiens adaptor-related protein complex 1, | NM_001283 | 1174 | 0.000171672 | −1 |
| | | | sigma 1 subunit (AP1S1), mRNA. | | | | |
| 48 | 2680010 | EPC1 | Homo sapiens enhancer of polycomb homolog 1 | NM_025209 | 80314 | 0.000552857 | −1 |
| | | | (Drosophila)(EPC1), mRNA. | | | | |
| 49 | 2690609 | CUTA | Homo sapiens cutA divalent cation tolerance | NM_001014433 | 51596 | 0.000701804 | −1 |
| | | | homolog (E. coli)(CUTA), transcript variant 1, | | | | |
| | | | mRNA. | | | | |
| 50 | 2710544 | C3orf37 | Homo sapiens chromosome 3 open reading frame | NM_001006109 | 56941 | 0.000548537 | −1 |
| | | | 37 (C3orf37), transcript variant 1, mRNA. | | | | |
| 51 | 2760563 | EIF2B1 | Homo sapiens eukaryotic translation initiation | NM_001414 | 1967 | 0.000638551 | 1 |
| | | | factor 2B, subunit 1 alpha, 26 kDa (EIF2B1), | | | | |
| | | | mRNA. | | | | |
| 52 | 2850100 | DTX3L | Homo sapiens deltex 3-like (Drosophila)(DTX3L), | NM_138287 | 151636 | 0.000689534 | −1 |
| | | | mRNA. | | | | |
| 53 | 2850377 | ITPR2 | Homo sapiens inositol 1,4,5-triphosphate receptor, | NM_002223 | 3709 | 0.000636057 | 1 |
| | | | type 2 (ITPR2), mRNA. | | | | |
| 54 | 2940224 | APH1A | Homo sapiens anterior pharynx defective 1 | NM_001077628 | 51107 | 0.000390857 | −1 |
| | | | homolog A (C. elegans )(APH1A), transcript | | | | |
| | | | variant 1, mRNA. | | | | |
| 55 | 3120301 | L3MBTL2 | Homo sapiens I(3)mbt-like 2 (Drosophila) | NM_031488 | 83746 | 0.000296114 | −1 |
| | | | (L3MBTL2), mRNA | | | | |
| 56 | 3140039 | CYB5R4 | Homo sapiens cytochrome b5 reductase 4 | NM_016230 | 51167 | 4.50809E−05 | 1 |
| | | | (CYB5R4), mRNA. | | | | |
| 57 | 3140093 | LZTR1 | Homo sapiens leucine-zipper-like transcription | NM_006767 | 8216 | 0.000628324 | −1 |
| | | | regulator 1 (LZTR1), mRNA. | | | | |
| 58 | 3180041 | TOR1AIP1 | Homo sapiens torsin A interacting protein 1 | NM_015602 | 26092 | 0.000606148 | −1 |
| | | | (TOR1AIP1), mRNA. | | | | |
| 59 | 3290162 | LAMP2 | Homo sapiens lysosomal-associated membrane | NM_001122606 | 3920 | 8.05131E−05 | −1 |
| | | | protein 2 (LAMP2), transcript variant C, mRNA. | | | | |
| 60 | 3290296 | ANKDD1A | Homo sapiens ankyrin repeat and death domain | NM_182703 | 348094 | 0.000470888 | −1 |
| | | | containing 1A (ANKDD1A), mRNA. | | | | |
| 61 | 3360364 | MORC2 | Homo sapiens MORC family CW-type zinc finger | NM_014941 | 22880 | 0.000389771 | −1 |
| | | | 2 (MORC2), mRNA. | | | | |
| 62 | 3360433 | IGF2BP3 | Homo sapiens insulin-like growth factor 2 mRNA | NM_006547 | 10643 | 0.000454733 | 1 |
| | | | binding protein 3 (IGF2BP3), mRNA | | | | |
| 63 | 3370402 | LOC401284 | PREDICTED: Homo sapiens hypothetical | XM_379454 | NA | 0.000393422 | 1 |
| | | | LOC401284 (LOC401284), mRNA. | | | | |
| 64 | 3460189 | STXBP5 | Homo sapiens syntaxin binding protein 5 | NM_139244 | 134957 | 0.000187189 | 1 |
| | | | (tomosyn) (STXBP5), transcript variant 1, mRNA. | | | | |
| 65 | 3460674 | SRPK1 | Homo sapiens SRSF protein kinase 1 (SRPK1), | NM_003137 | 6732 | 3.88823E−05 | 1 |
| | | | transcript variant 1, mRNA. | | | | |
| 66 | 3520082 | RUVBL1 | Homo sapiens RuvB-like 1 (E. coli)(RUVBL1), | NM_003707 | 8607 | 0.000217416 | −1 |
| | | | mRNA | | | | |
| 67 | 3610504 | GNE | Homo sapiens glucosamine (UDP-N-acetyl)-2- | NM_005476 | 10020 | 3.36864E−05 | −1 |
| | | | epimerase/N-acetylmannosamine kinase (GNE), | | | | |
| | | | transcript variant 2, mRNA. | | | | |
| 68 | 3780689 | NT5C3 | Homo sapiens 5′-nucleotidase, cytosolic III | NM_001002009 | 51251 | 6.68713E−06 | 1 |
| | | | (NT5C3), transcript variant 2, mRNA. | | | | |
| 69 | 3800270 | CCR2 | Homo sapiens chemokine (C-C motif) receptor 2 | NM_001123041 | 1231 | 0.000167225 | −1 |
| | | | (CCR2), transcript variant A, mRNA. | | | | |
| 70 | 3830341 | LYRM1 | Homo sapiens LYR motif containing 1 (LYRM1), | NM_020424 | 57149 | 0.000124551 | −1 |
| | | | transcript variant 1, mRNA. | | | | |
| 71 | 3830390 | KIAA0692 | PREDICTED: Homo sapiens KIAA0692 protein, | XM_930898 | NA | 0.000774647 | 1 |
| | | | transcript variant 12 (KIAA0692), mRNA. | | | | |
| 72 | 3870754 | FBXO28 | Homo sapiens F-box protein 28 (FBXO28), | NM_015176 | 23219 | 0.000764424 | −1 |
| | | | transcript variant 1, mRNA. | | | | |
| 73 | 3990176 | PROSC | Homo sapiens proline synthetase co-transcribed | NM_007198 | 11212 | 0.000546284 | −1 |
| | | | homolog (bacterial) (PROSC), mRNA. | | | | |
| 74 | 3990639 | IL23A | Homo sapiens interleukin 23, alpha subunit p19 | NM_016584 | 51561 | 0.000463322 | −1 |
| | | | (IL23A), mRNA. | | | | |
| 75 | 4010048 | ACOX1 | Homo sapiens acyl-CoA oxidase 1, palmitoyl | NM_004035 | 51 | 0.000452803 | 1 |
| | | | (ACOX1), transcript variant 1, mRNA. | | | | |
| 76 | 4050195 | NA | Homo sapiens genomic DNA; cDNA | AL080095 | NA | 0.000112435 | 1 |
| | | | DKFZp564O0862 (from clone DKFZp564O0862). | | | | |
| 77 | 4050270 | NA | UI-E-CK1-afm-g-09-0-UI.s2 UI-E-CK1 Homo | BM668555.1 | NA | 0.000226572 | 1 |
| | | | sapiens cDNA clone UI-E-CK1-afm-g-09-0-UI 3-, | | | | |
| | | | mRNA sequence | | | | |
| 78 | 4060131 | LPXN | Homo sapiens leupaxin (LPXN), transcript variant | NM_004811 | 9404 | 0.000789377 | −1 |
| | | | 2, mRNA. | | | | |
| 79 | 4060138 | NA | PREDICTED: Homo sapiens similar to | XM_941904 | NA | 0.000295927 | −1 |
| | | | Transcriptional regulator ATRX (ATP-dependent | | | | |
| | | | helicase ATRX) (X-linked helicase II) (X-linked | | | | |
| | | | nuclear protein) (XNP) (Znf-HX) (LOC652455), | | | | |
| | | | mRNA. | | | | |
| 80 | 4060605 | CD44 | Homo sapiens CD44 molecule (Indian blood | NM_000610 | 960 | 0.000351524 | −1 |
| | | | group) (CD44), transcript variant 1, mRNA. | | | | |
| 81 | 4220138 | SDHAF1 | Homo sapiens succinate dehydrogenase complex | NM_001042631 | 644096 | 0.000531803 | −1 |
| | | | assembly factor 1 (SDHAF1), nuclear gene | | | | |
| | | | encoding mitochondrial protein, mRNA. | | | | |
| 82 | 4230253 | MLL5 | Homo sapiens myeloid/lymphoid or mixed-lineage | NM_018682 | 55904 | 0.000183207 | −1 |
| | | | leukemia 5 (trithorax homolog, Drosophila) | | | | |
| | | | (MLL5), transcript variant 2, mRNA. | | | | |
| 83 | 4260102 | NA | UI-H-BI3-ajz-b-11-0-UI.s1 NCI_CGAP_Sub5 | AW444880.1 | NA | 0.000278766 | 1 |
| | | | Homo sapiens cDNA clone IMAGE: 2733285 3, | | | | |
| | | | mRNA sequence | | | | |
| 84 | 4280047 | RNF13 | Homo sapiens ring finger protein 13 (RNF13), | NM_183383 | 11342 | 0.000775091 | 1 |
| | | | transcript variant 3, mRNA. | | | | |
| 85 | 4280056 | C12orf49 | Homo sapiens chromosome 12 open reading | NM_024738 | 79794 | 0.000774408 | 1 |
| | | | frame 49 (C12orf49), mRNA. | | | | |
| 86 | 4280332 | DDX24 | Homo sapiens DEAD (Asp-Glu-Ala-Asp) box | NM_020414 | 57062 | 0.000120548 | −1 |
| | | | polypeptide 24 (DDX24), mRNA. | | | | |
| 87 | 4280373 | SVIL | Homo sapiens supervillin (SVIL), transcript variant | NM_003174 | 6840 | 0.000309748 | 1 |
| | | | 1, mRNA. | | | | |
| 88 | 4290477 | NA | Homo sapiens sperm associated antigen 9 | NM_172345 | NA | 0.000362839 | 1 |
| | | | (SPAG9), transcript variant 2, mRNA. | | | | |
| 89 | 4540082 | PHF19 | Homo sapiens PHD finger protein 19 (PHF19), | NM_001009936 | 26147 | 0.000502643 | −1 |
| | | | transcript variant 2, mRNA. | | | | |
| 90 | 4560039 | PRUNE | Homo sapiens prune homolog (Drosophila) | NM_021222 | 58497 | 4.24358E−05 | 1 |
| | | | (PRUNE), mRNA. | | | | |
| 91 | 4570730 | LOC645232 | PREDICTED: Homo sapiens hypothetical protein | XM_928271 | NA | 0.000739672 | −1 |
| | | | LOC645232 (LOC645232), mRNA. | | | | |
| 92 | 4640044 | BCL6 | Homo sapiens B-cell CLL/lymphoma 6 (zinc finger | NM_138931 | 604 | 0.000484396 | 1 |
| | | | protein 51) (BCL6), transcript variant 2, mRNA. | | | | |
| 93 | 4730195 | HIST1H4H | Homo sapiens histone cluster 1, H4h | NM_003543 | 8365 | 3.12382E−05 | 1 |
| | | | (HIST1H4H), mRNA. | | | | |
| 94 | 4730577 | NA | UI-E-EJ1-aka-f-15-0-UI.s1 UI-E-EJ1 Homo | CK300859.1 | NA | 0.000578904 | −1 |
| | | | sapiens cDNA clone UI-E-EJ1-aka-f-15-0-UI 3-, | | | | |
| | | | mRNA sequence | | | | |
| 95 | 4760543 | NUP62 | Homo sapiens nucleoporin 62 kDa (NUP62), | NM_012346 | 23636 | 0.000292124 | −1 |
| | | | transcript variant 4, mRNA. | | | | |
| 96 | 4810204 | CYSLTR1 | Homo sapiens cysteinyl leukotriene receptor 1 | NM_006639 | 10800 | 8.94451E−05 | 1 |
| | | | (CYSLTR1), mRNA. | | | | |
| 97 | 4850711 | LOC644474 | PREDICTED: Homo sapiens hypothetical protein | XM_930098 | NA | 0.000527041 | −1 |
| | | | LOC644474 (LOC644474), mRNA. | | | | |
| 98 | 4900053 | PUF60 | Homo sapiens poly-U binding splicing factor | NM_014281 | 22827 | 0.000279673 | −1 |
| | | | 60 KDa (PUF60), transcript variant 2, mRNA. | | | | |
| 99 | 4920142 | LAMP2 | Homo sapiens lysosomal-associated membrane | NM_001122606 | 3920 | 0.000430427 | 1 |
| | | | protein 2 (LAMP2), transcript variant C, mRNA. | | | | |
| 100 | 4920575 | ZNF740 | Homo sapiens zinc finger protein 740 (ZNF740), | NM_001004304 | 283337 | 0.00068817 | −1 |
| | | | mRNA. | | | | |
| 101 | 5090477 | PIP4K2B | Homo sapiens phosphatidylinositol-5-phosphate | NM_003559 | 8396 | 2.45489E−05 | −1 |
| | | | 4-kinase, type II, beta (PIP4K2B), mRNA | | | | |
| 102 | 5290289 | YIPF4 | Homo sapiens Yip1 domain family, member 4 | NM_032312 | 84272 | 0.000514659 | 1 |
| | | | (YIPF4), mRNA. | | | | |
| 103 | 5290452 | CPEB3 | Homo sapiens cytoplasmic polyadenylation | NM_014912 | 22849 | 0.000159172 | 1 |
| | | | element binding protein 3 (CPEB3), transcript | | | | |
| | | | variant 1, mRNA. | | | | |
| 104 | 5310754 | METTL13 | Homo sapiens methyltransferase like 13 | NM_001007239 | 51603 | 0.000419383 | −1 |
| | | | (METTL13), transcript variant 3, mRNA. | | | | |
| 105 | 5340246 | CD9 | Homo sapiens CD9 molecule (CD9), mRNA. | NM_001769 | 928 | 0.000216455 | −1 |
| 106 | 5390131 | MCM3AP | Homo sapiens minichromosome maintenance | NM_003906 | 8888 | 0.000419553 | −1 |
| | | | complex component 3 associated protein | | | | |
| | | | (MCM3AP), mRNA. | | | | |
| 107 | 5390504 | BIRC3 | Homo sapiens baculoviral IAP repeat containing 3 | NM_001165 | 330 | 0.000174761 | 1 |
| | | | (BIRC3), transcript variant 1, mRNA. | | | | |
| 108 | 5490064 | OTUD1 | PREDICTED: Homo sapiens OTU domain | XM_939698 | NA | 0.000361512 | −1 |
| | | | containing 1 (OTUD1), mRNA. | | | | |
| 109 | 5690037 | PRSS50 | Homo sapiens protease, serine, 50 (PRSS50), | NM_013270 | 29122 | 0.000112016 | 1 |
| | | | mRNA. | | | | |
| 110 | 5720681 | TIPARP | Homo sapiens TCDD-inducible poly(ADP-ribose) | NM_015508 | 25976 | 0.000373542 | −1 |
| | | | polymerase (TIPARP), transcript variant 2, mRNA. | | | | |
| 111 | 5860196 | NA | UI-E-CI1-afs-e-04-0-UI.s1 UI-E-CI1 Homo sapiens | BU733214.1 | NA | 0.000141309 | 1 |
| | | | cDNA clone UI-E-CI1-afs-e-04-0-UI 3-, mRNA | | | | |
| | | | sequence | | | | |
| 112 | 5860400 | HIST1H2AE | Homo sapiens histone cluster 1, H2ae | NM_021052 | 3012 | 0.000116423 | 1 |
| | | | (HIST1H2AE), mRNA. | | | | |
| 113 | 5860500 | EIF2C3 | Homo sapiens eukaryotic translation initiation | NM_024852 | 192669 | 0.000613944 | −1 |
| | | | factor 2C, 3 (EIF2C3), transcript variant 1, mRNA. | | | | |
| 114 | 5900156 | TUBA1B | Homo sapiens tubulin, alpha 1b (TUBA1B), | NM_006082 | 10376 | 0.000118183 | −1 |
| | | | mRNA. | | | | |
| 115 | 5910091 | ANKK1 | Homo sapiens ankyrin repeat and kinase domain | NM_178510 | 255239 | 0.000308507 | −1 |
| | | | containing 1 (ANKK1), mRNA. | | | | |
| 116 | 5910682 | LOC348645 | Homo sapiens hypothetical protein LOC348645 | NM_198851 | NA | 0.000643342 | −1 |
| | | | (LOC348645), mRNA. | | | | |
| 117 | 5960128 | TAF15 | Homo sapiens TAF15 RNA polymerase II, TATA | NM_003487 | 8148 | 0.000190658 | −1 |
| | | | box binding protein (TBP)-associated factor, | | | | |
| | | | 68 kDa (TAF15), transcript variant 2, mRNA. | | | | |
| 118 | 6020402 | SRP68 | Homo sapiens signal recognition particle 68 kDa | NM_014230 | 6730 | 0.000749867 | −1 |
| | | | (SRP68), mRNA. | | | | |
| 119 | 6110088 | ABCA1 | Homo sapiens ATP-binding cassette, sub-family A | NM_005502 | 19 | 0.000514553 | 1 |
| | | | (ABC1), member 1 (ABCA1), mRNA. | | | | |
| 120 | 6110537 | LOC284701 | PREDICTED: Homo sapiens similar to | XM_931928 | NA | 0.000606685 | −1 |
| | | | hypothetical protein LOC284701, transcript variant | | | | |
| | | | 2 (LOC642816), mRNA. | | | | |
| 121 | 6110768 | ATIC | Homo sapiens 5-aminoimidazole-4-carboxamide | NM_004044 | 471 | 0.000286296 | −1 |
| | | | ribonucleotide formyltransferase/IMP | | | | |
| | | | cyclohydrolase (ATIC), mRNA. | | | | |
| 122 | 6180427 | GPR160 | Homo sapiens G protein-coupled receptor 160 | NM_014373 | 26996 | 0.000312842 | −1 |
| | | | (GPR160), mRNA. | | | | |
| 123 | 6200563 | ZNF654 | Homo sapiens zinc finger protein 654 (ZNF654), | NM_018293 | 55279 | 0.000270097 | 1 |
| | | | mRNA. | | | | |
| 124 | 6220022 | RNF38 | Homo sapiens ring finger protein 38 (RNF38), | NM_022781 | 152006 | 0.000362573 | −1 |
| | | | transcript variant 1, mRNA. | | | | |
| 125 | 6220450 | DHRS9 | Homo sapiens dehydrogenase/reductase (SDR | NM_005771 | 10170 | 0.00014906 | 1 |
| | | | family) member 9 (DHRS9), transcript variant 1, | | | | |
| | | | mRNA. | | | | |
| 126 | 6270128 | CD40LG | Homo sapiens CD40 ligand (CD40LG), mRNA. | NM_000074 | 959 | 0.000197683 | −1 |
| 127 | 6270301 | AP1S1 | Homo sapiens adaptor-related protein complex 1, | NM_001283 | 1174 | 8.37111E−05 | −1 |
| | | | sigma 1 subunit (AP1S1), mRNA. | | | | |
| 128 | 6280343 | EEF2K | Homo sapiens eukaryotic elongation factor-2 | NM_013302 | 29904 | 0.000263638 | −1 |
| | | | kinase (EEF2K), mRNA. | | | | |
| 129 | 6290458 | ZNF200 | Homo sapiens zinc finger protein 200 (ZNF200), | NM_003454 | 7752 | 0.000253711 | 1 |
| | | | transcript variant 1, mRNA. | | | | |
| 130 | 6350452 | APAF1 | Homo sapiens apoptotic peptidase activating | NM_001160 | 317 | 0.000611935 | 1 |
| | | | factor 1 (APAF1), transcript variant 2, mRNA. | | | | |
| 131 | 6350608 | MYLK | Homo sapiens myosin light chain kinase (MYLK), | NM_053025 | 4638 | 8.97415E−05 | −1 |
| | | | transcript variant 1, mRNA. | | | | |
| 132 | 6380598 | IMP4 | Homo sapiens IMP4, U3 small nucleolar | NM_033416 | 92856 | 0.000519664 | −1 |
| | | | ribonucleoprotein, homolog (yeast) (IMP4), | | | | |
| | | | mRNA. | | | | |
| 133 | 6420692 | RSBN1L | Homo sapiens round spermatid basic protein 1- | NM_198467 | 222194 | 0.0005904 | 1 |
| | | | like (RSBN1L), mRNA. | | | | |
| 134 | 6520333 | LOC652759 | PREDICTED: Homo sapiens similar to F-box and | XM_942392 | NA | 0.000152094 | −1 |
| | | | WD-40 domain protein 10 (LOC652759), mRNA. | | | | |
| 135 | 6550520 | LYSMD2 | Homo sapiens LysM, putative peptidoglycan- | NM_153374 | 256586 | 0.000356986 | −1 |
| | | | binding, domain containing 2 (LYSMD2), transcript | | | | |
| | | | variant 1, mRNA. | | | | |
| 136 | 6580445 | ENKUR | Homo sapiens enkurin, TRPC channel interacting | NM_145010 | 219670 | 0.000361628 | −1 |
| | | | protein (ENKUR), mRNA. | | | | |
| 137 | 6590278 | AP3M1 | Homo sapiens adaptor-related protein complex 3, | NM_012095 | 26985 | 0.000105537 | −1 |
| | | | mu 1 subunit (AP3M1), transcript variant 2, | | | | |
| | | | mRNA | | | | |
| 138 | 6590386 | FN3KRP | Homo sapiens fructosamine 3 kinase related | NM_024619 | 79672 | 0.000254624 | −1 |
| | | | protein (FN3KRP), mRNA. | | | | |
| 139 | 6660097 | QKI | Homo sapiens quaking homolog, KH domain RNA | NM_006775 | 9444 | 9.41329E−05 | −1 |
| | | | binding (mouse) (QKI), transcript variant 1, | | | | |
| | | | mRNA. | | | | |
| 140 | 6760441 | OSBP | Homo sapiens oxysterol binding protein (OSBP), | NM_002556 | 5007 | 0.000222086 | −1 |
| | | | mRNA | | | | |
| 141 | 6940524 | PDE5A | Homo sapiens phosphodiesterase 5A, cGMP- | NM_001083 | 8654 | 0.000118562 | 1 |
| | | | specific (PDE5A), transcript variant 1, mRNA. | | | | |
| 142 | 6960746 | GIMAP5 | Homo sapiens GTPase, IMAP family member 5 | NM_018384 | 55340 | 0.000373699 | −1 |
| | | | (GIMAP5), mRNA. | | | | |
| 143 | 6980070 | B4GALT5 | Homo sapiens UDP-Gal:betaGlcNAc beta 1,4- | NM_004776 | 9334 | 0.000353132 | 1 |
| | | | galactosyltransferase, polypeptide 5 (B4GALT5), | | | | |
| | | | mRNA. | | | | |
| 144 | 6980129 | PGK1 | Homo sapiens phosphoglycerate kinase 1 | NM_000291 | 5230 | 0.000771983 | −1 |
| | | | (PGK1), mRNA. | | | | |
| 145 | 6980274 | NA | 603176844F1 NIH_MGC_121 Homo sapiens | BI915661.1 | NA | 0.000455734 | 1 |
| | | | cDNA clone IMAGE: 5241250 5-, mRNA sequence | | | | |
| 146 | 6980609 | LRRTM1 | Homo sapiens leucine rich repeat transmembrane | NM_178839 | 347730 | 0.000250817 | −1 |
| | | | neuronal 1 (LRRTM1), mRNA. | | | | |
| 147 | 7040187 | ARRDC4 | Homo sapiens arrestin domain containing 4 | NM_183376 | 91947 | 5.48071E−05 | −1 |
| | | | (ARRDC4), mRNA. | | | | |
| 148 | 7050543 | COQ6 | Homo sapiens coenzyme Q6 homolog, | NM_182476 | 51004 | 0.000756562 | −1 |
| | | | monooxygenase (S. cerevisiae) (COQ6), nuclear | | | | |
| | | | gene encoding mitochondrial protein, transcript | | | | |
| | | | variant 1, mRNA. | | | | |
| 149 | 7100136 | SLC36A1 | Homo sapiens solute carrier family 36 | NM_078483 | 206358 | 0.000359438 | 1 |
| | | | (proton/amino acid symporter), member 1 | | | | |
| | | | (SLC36A1), mRNA. | | | | |
| 150 | 7100520 | WHSC1 | Homo sapiens Wolf-Hirschhorn syndrome | NM_001042424 | 7468 | 0.000181408 | 1 |
| | | | candidate 1 (WHSC1), transcript variant 10, | | | | |
| | | | mRNA. | | | | |
| 151 | 7150634 | MYO9A | Homo sapiens myosin IXA (MYO9A), mRNA. | NM_006901 | 4649 | 0.000141291 | −1 |
| 152 | 7160296 | PDCD11 | Homo sapiens programmed cell death 11 | NM_014976 | 22984 | 0.000581747 | −1 |
| | | | (PDCD11), mRNA. | | | | |
| 153 | 7160767 | UBE2Z | Homo sapiens ubiquitin-conjugating enzyme E2Z | NM_023079 | 65264 | 0.000605771 | −1 |
| | | | (UBE2Z), mRNA. | | | | |
| 154 | 7200681 | KIAA1618 | PREDICTED: Homo sapiens KIAA1618 | XM_941239 | NA | 0.000132256 | −1 |
| | | | (KIAA1618), mRNA. | | | | |
| 155 | 7210372 | UGCGL1 | Homo sapiens UDP-glucose ceramide | NM_001025777 | 56886 | 1.21875E−05 | −1 |
| | | | glucosyltransferase-like 1 (UGCGL1), transcript | | | | |
| | | | variant 2, mRNA. | | | | |
| 156 | 7320047 | SAMHD1 | Homo sapiens SAM domain and HD domain 1 | NM_015474 | 25939 | 0.000542851 | −1 |
| | | | (SAMHD1), mRNA. | | | | |
| 157 | 7380274 | ZMYM6 | Homo sapiens zinc finger, MYM-type 6 (ZMYM6), | NM_007167 | 9204 | 0.000385861 | −1 |
| | | | mRNA. | | | | |
| 158 | 7380288 | ANAPC5 | Homo sapiens anaphase promoting complex | NM_016237 | 51433 | 0.000593428 | −1 |
| | | | subunit 5 (ANAPC5), transcript variant 1, mRNA. | | | | |
| 159 | 7550537 | SLC25A5 | Homo sapiens solute carrier family 25 | NM_001152 | 292 | 7.90327E−05 | −1 |
| | | | (mitochondrial carrier; adenine nucleotide | | | | |
| | | | translocator), member 5 (SLC25A5), nuclear gene | | | | |
| | | | encoding mitochondrial protein, mRNA. | | | | |
| 160 | 7570603 | RAB31 | Homo sapiens RAB31, member RAS oncogene | NM_006868 | 11031 | 0.000162417 | 1 |
| | | | family (RAB31), mRNA. | | | | |
| 161 | 7650379 | TMEM154 | Homo sapiens transmembrane protein 154 | NM_152680 | 201799 | 0.000127518 | −1 |
| | | | (TMEM154), mRNA. | | | | |

TABLE 3b

| | | | | | | | Over-(1)/ |
|---|---|---|---|---|---|---|---|
| SEQ | | | | | | | under- |
| ID | | | | | | | (−1) |
| NO. | ID | Symbol | Gene name | Refseq | Score | p-Value | expression |
| 162 | 6960440 | DEFA4 | Homo sapiens defensin, alpha 4, corticostatin | NM_001925.1 | 59725 | 1.72E−05 | 1 |
| | | | (DEFA4), mRNA | | | | |
| 163 | 10279 | S100A12 | Homo sapiens S100 calcium binding protein | NM_005621.1 | 59521 | 3.94E−13 | 1 |
| | | | A12 (S100A12), mRNA | | | | |
| 164 | 990097 | CEACAM8 | Homo sapiens carcinoembryonic antigen- | NM_001816.3 | 58964 | 2.70E−05 | 1 |
| | | | related cell adhesion molecule 8 (CEACAM8), | | | | |
| | | | mRNA | | | | |
| 165 | 1090427 | LOC653600 | PREDICTED: Homo sapiens similar to | XM_928349.1 | 57913 | 6.94E−04 | 1 |
| | | | Neutrophil defensin 1 precursor (HNP-1) (HP- | | | | |
| | | | 1) (HP1) (Defensin, alpha 1) (LOC653600), | | | | |
| | | | mRNA | | | | |
| 166 | 1470554 | ELA2 | Homo sapiens elastase, neutrophil expressed | NM_001972.2 | 52995 | 2.43E−02 | 1 |
| | | | (ELANE), mRNA | | | | |
| 167 | 6980537 | HS.291319 | Homo sapiens mRNA; cDNA | CR627122.1 | 51732 | 9.44E−08 | 1 |
| | | | DKFZp779M2422 (from clone | | | | |
| | | | DKFZp779M2422) | | | | |
| 168 | 6860754 | ARG1 | Homo sapiens arginase, liver (ARG1), | NM_000045.3 | 50327 | 1.09E−05 | 1 |
| | | | transcript variant 2, mRNA | | | | |
| 169 | 2810040 | APOBEC3A | Homo sapiens apolipoprotein B mRNA editing | NM_145699.3 | 49394 | 6.28E−10 | 1 |
| | | | enzyme, catalytic polypeptide-like 3A | | | | |
| | | | (APOBEC3A), transcript variant 1, mRNA | | | | |
| 170 | 1580259 | LOC389787 | PREDICTED: Homo sapiens similar to | XM_497072.2 | 48009 | 8.32E−12 | 1 |
| | | | Translationally-controlled tumor protein | | | | |
| | | | (TCTP) (p23) (Histamine-releasing factor) | | | | |
| | | | (HRF) (Fortilin) (LOC389787), mRNA | | | | |
| 171 | 6960554 | LCN2 | Homo sapiens lipocalin 2 (LCN2), mRNA | NM_005564.3 | 47528 | 1.44E−03 | 1 |
| 172 | 4390692 | HLA-DRB5 | Homo sapiens major histocompatibility | NM_002125.3 | 47088 | 0.001066358 | −1 |
| | | | complex, class II, DR beta 5 (HLA-DRB5), | | | | |
| | | | mRNA | | | | |
| 173 | 4250035 | RAP1GAP | Homo sapiens RAP1 GTPase activating | NM_002885.2 | 46445 | 8.40E−01 | 1 |
| | | | protein (RAP1GAP), transcript variant 3, | | | | |
| | | | mRNA | | | | |
| 174 | 1240044 | CEACAM6 | Homo sapiens carcinoembryonic antigen- | NM_002483.4 | 45970 | 2.46E−04 | 1 |
| | | | related cell adhesion molecule 6 (non-specific | | | | |
| | | | cross reacting antigen) (CEACAM6), mRNA | | | | |
| 175 | 3400551 | MS4A3 | Homo sapiens membrane-spanning 4- | NM_006138.4 | 44765 | 2.44E−05 | 1 |
| | | | domains, subfamily A, member 3 | | | | |
| | | | (hematopoietic cell-specific) (MS4A3), | | | | |
| | | | transcript variant 1, mRNA | | | | |
| 176 | 4390242 | DEFA1 | Homo sapiens defensin, alpha 1 (DEFA1), | NM_004084.3 | 44468 | 9.03E−08 | 1 |
| | | | mRNA | | | | |
| 177 | 6330376 | CA1 | Homo sapiens carbonic anhydrase I (CA1), | NM_001738.3 | 40459 | 4.93E−01 | 1 |
| | | | transcript variant 2, mRNA | | | | |
| 178 | 830619 | CTSG | Homo sapiens cathepsin G (CTSG), mRNA | NM_001911.2 | 39180 | 1.30E−01 | 1 |
| 179 | 4060066 | ITGA2B | Homo sapiens integrin, alpha 2b (platelet | NM_000419.3 | 37796 | 3.61E−08 | 1 |
| | | | glycoprotein IIb of IIb/IIIa complex, antigen | | | | |
| | | | CD41) (ITGA2B), mRNA | | | | |
| 180 | 4050286 | LOC645671 | PREDICTED: Homo sapiens similar to | XM_928682.1 | 36247 | 6.24E−11 | 1 |
| | | | CG15133-PA (LOC645671), mRNA | | | | |
| 181 | 4560133 | ANXA3 | Homo sapiens annexin A3 (ANXA3), mRNA | NM_005139.2 | 36109 | 3.05E−10 | 1 |
| 182 | 70338 | SP110 | Homo sapiens SP110 nuclear body protein | NM_004510.3 | 35829 | 3.94E−13 | 1 |
| | | | (SP110), transcript variant b, mRNA | | | | |
| 183 | 5900072 | LOC347376 | PREDICTED: Homo sapiens similar to H3 | XM_937928.2 | 35717 | 3.54E−16 | 1 |
| | | | histone, family 3B (LOC347376), mRNA | | | | |
| 184 | 6350364 | PPBP | Homo sapiens pro-platelet basic protein | NM_002704.3 | 35608 | 1.04E−12 | 1 |
| | | | (chemokine (C—X—C motif) ligand 7) (PPBP), | | | | |
| | | | mRNA | | | | |
| 185 | 160348 | RNASE3 | Homo sapiens ribonuclease, RNase A family, | NM_002935.2 | 34612 | 1.32E−03 | 1 |
| | | | 3 (RNASE3), mRNA | | | | |
| 186 | 1190349 | EIF2AK2 | Homo sapiens eukaryotic translation initiation | NM_002759.3 | 34259 | 8.30E−09 | 1 |
| | | | factor 2-alpha kinase 2 (EIF2AK2), transcript | | | | |
| | | | variant 1, mRNA | | | | |
| 187 | 5080398 | TLR1 | Homo sapiens toll-like receptor 1 (TLR1), | NM_003263.3 | 34155 | 2.70E−14 | 1 |
| | | | mRNA | | | | |
| 188 | 2370524 | TNFAIP6 | Homo sapiens tumor necrosis factor, alpha- | NM_007115.3 | 33533 | 1.24E−16 | 1 |
| | | | induced protein 6 (TNFAIP6), mRNA | | | | |
| 189 | 6400736 | CAMP | Homo sapiens cathelicidin antimicrobial | NM_004345.4 | 32959 | 1.42E−04 | 1 |
| | | | peptide (CAMP), mRNA | | | | |
| 190 | 520646 | BLVRB | Homo sapiens biliverdin reductase B (flavin | NM_000713.2 | 31869 | 1.15E−05 | 1 |
| | | | reductase (NADPH)) (BLVRB), mRNA | | | | |
| 191 | 6180161 | LOC389293 | PREDICTED: Homo sapiens similar to HESB | XM_371741.5 | 31103 | 1.43E−07 | 1 |
| | | | like domain containing 2, transcript variant 1 | | | | |
| | | | (LOC389293), mRNA | | | | |
| 192 | 360066 | VPREB3 | Homo sapiens pre-B lymphocyte 3 (VPREB3), | NM_013378.2 | 30363 | 5.19E−14 | −1 |
| | | | mRNA | | | | |
| 193 | 7570079 | IL7R | Homo sapiens interleukin 7 receptor (IL7R), | NM_002185.2 | 29915 | 9.94E−06 | 1 |
| | | | mRNA | | | | |
| 194 | 2340110 | MGC13057 | Homo sapiens chromosome 2 open reading | NM_032321.2 | 28889 | 7.60E−07 | 1 |
| | | | frame 88 (C2orf88), transcript variant 4, | | | | |
| | | | mRNA | | | | |
| 195 | 4120707 | RPL23 | Homo sapiens ribosomal protein L23 (RPL23), | NM_000978.3 | 28873 | 7.89E−03 | 1 |
| | | | mRNA | | | | |
| 196 | 520228 | UBE2H | Homo sapiens ubiquitin-conjugating enzyme | NM_182697.2 | 28757 | 3.39E−08 | 1 |
| | | | E2H (UBE2H), transcript variant 2, mRNA | | | | |
| 197 | 7650678 | FAM46C | Homo sapiens family with sequence similarity | NM_017709.3 | 28674 | 2.30E−03 | 1 |
| | | | 46, member C (FAM46C), mRNA | | | | |
| 198 | 430328 | ERAF | Homo sapiens alpha hemoglobin stabilizing | NM_016633.2 | 28507 | 2.01E−04 | 1 |
| | | | protein (AHSP), mRNA | | | | |
| 199 | 3170241 | FECH | Homo sapiens ferrochelatase (FECH), nuclear | NM_000140.3 | 28394 | 4.30E−02 | 1 |
| | | | gene encoding mitochondrial protein, | | | | |
| | | | transcript variant 2, mRNA | | | | |
| 200 | 6620711 | RSAD2 | Homo sapiens radical S-adenosyl methionine | NM_080657.4 | 28378 | 9.77E+00 | 1 |
| | | | domain containing 2 (RSAD2), mRNA | | | | |
| 201 | 5050075 | FTHL12 | Homo sapiens ferritin, heavy polypeptide-like | NR_002205.1 | 28205 | 9.79E−13 | 1 |
| | | | 12 (FTHL12) on chromosome 9 | | | | |
| 202 | 2680273 | ZFP36L1 | Homo sapiens zinc finger protein 36, C3H | NM_004926.3 | 28122 | 3.02E−12 | 1 |
| | | | type-like 1 (ZFP36L1), transcript variant 1, | | | | |
| | | | mRNA | | | | |
| 203 | 610148 | BPI | Homo sapiens bactericidal/permeability- | NM_001725.2 | 27817 | 1.10E−02 | 1 |
| | | | increasing protein (BPI), mRNA | | | | |
| 204 | 2650440 | FTHL2 | Homo sapiens ferritin, heavy polypeptide-like | NR_002200.1 | 27398 | 6.18E−12 | 1 |
| | | | 2 (FTHL2) on chromosome 1 | | | | |
| 205 | 4210414 | FTHL11 | Homo sapiens ferritin, heavy polypeptide-like | NR_002204.1 | 27394 | 3.18E−11 | 1 |
| | | | 11 (FTHL11) on chromosome 8 | | | | |
| 206 | 4120270 | YOD1 | Homo sapiens YOD1 OTU deubiquinating | NM_018566.3 | 27384 | 3.43E−02 | 1 |
| | | | enzyme 1 homolog (S. cerevisiae ) (YOD1), | | | | |
| | | | mRNA | | | | |
| 207 | 380307 | ACTR3 | Homo sapiens ARP3 actin-related protein 3 | NM_005721.3 | 27380 | 6.56E−15 | 1 |
| | | | homolog (yeast) (ACTR3), mRNA | | | | |
| 208 | 7400097 | TCN1 | Homo sapiens transcobalamin I (vitamin B12 | NM_001062.3 | 27071 | 1.37E−05 | 1 |
| | | | binding protein, R binder family) (TCN1), | | | | |
| | | | mRNA | | | | |
| 209 | 2760463 | LOC389293 | PREDICTED: Homo sapiens similar to HESB | XM_931683.2 | 26694 | 4.89E−05 | 1 |
| | | | like domain containing 2, transcript variant 2 | | | | |
| | | | (LOC389293), mRNA | | | | |
| 210 | 620324 | LOC647673 | PREDICTED: Homo sapiens similar to | XM_936731.1 | 26234 | 5.33E−11 | 1 |
| | | | Translationally-controlled tumor protein | | | | |
| | | | (TCTP) (p23) (Histamine-releasing factor) | | | | |
| | | | (HRF) (Fortilin) (LOC647673), mRNA | | | | |
| 211 | 580307 | KCTD12 | Homo sapiens potassium channel | NM_138444.3 | 26101 | 7.78E−11 | 1 |
| | | | tetramerisation domain containing 12 | | | | |
| | | | (KCTD12), mRNA | | | | |
| 212 | 6450692 | FAM104A | Homo sapiens family with sequence similarity | NM_032837.2 | 25977 | 6.22E−07 | 1 |
| | | | 104, member A (FAM104A), transcript variant | | | | |
| | | | 2, mRNA | | | | |
| 213 | 1260228 | PLSCR1 | Homo sapiens phospholipid scramblase 1 | NM_021105.2 | 25867 | 5.90E−15 | 1 |
| | | | (PLSCR1), mRNA | | | | |
| 214 | 4880717 | ACSL1 | Homo sapiens acyl-CoA synthetase long- | NM_001995.2 | 25230 | 1.29E−09 | 1 |
| | | | chain family member 1 (ACSL1), mRNA | | | | |
| 215 | 3520474 | GYPE | Homo sapiens glycophorin E (MNS blood | NM_002102.3 | 25090 | 1.37E−01 | 1 |
| | | | group) (GYPE), transcript variant 1, mRNA | | | | |
| 216 | 6350446 | BNIP3L | Homo sapiens BCL2/adenovirus E1B 19 kDa | NM_004331.2 | 25025 | 2.07E−01 | 1 |
| | | | interacting protein 3-like (BNIP3L), mRNA | | | | |
| 217 | 4880390 | SNAP23 | Homo sapiens synaptosomal-associated | NM_130798.2 | 24097 | 4.68E−10 | 1 |
| | | | protein, 23 kDa (SNAP23), transcript variant 2, | | | | |
| | | | mRNA | | | | |
| 218 | 6200221 | XK | Homo sapiens X-linked Kx blood group | NM_021083.2 | 24056 | 4.82E−03 | 1 |
| | | | (McLeod syndrome) (XK), mRNA | | | | |
| 219 | 4180564 | LOC388621 | PREDICTED: Homo sapiens similar to | XM_371243.5 | 23938 | 4.34E−06 | 1 |
| | | | ribosomal protein L21 (LOC388621), mRNA | | | | |
| 220 | 7200309 | FAM49B | Homo sapiens family with sequence similarity | NM_016623.4 | 23826 | 1.92E−16 | 1 |
| | | | 49, member B (FAM49B), transcript variant 2, | | | | |
| | | | mRNA | | | | |
| 221 | 1070181 | SUMO2 | #NV | #NV | 23680 | 7.76E−09 | 1 |
| 222 | 1450309 | RNASE2 | Homo sapiens ribonuclease, RNase A family, | NM_002934.2 | 23623 | 2.14E−06 | 1 |
| | | | 2 (liver, eosinophil-derived neurotoxin) | | | | |
| | | | (RNASE2), mRNA | | | | |
| 223 | 6250037 | HBD | Homo sapiens hemoglobin, delta (HBD), | NM_000519.3 | 23567 | 3.24E−06 | 1 |
| | | | mRNA | | | | |
| 224 | 3830138 | OSBPL8 | Homo sapiens oxysterol binding protein-like 8 | NM_020841.4 | 23509 | 1.90E−08 | 1 |
| | | | (OSBPL8), transcript variant 1, mRNA | | | | |
| 225 | 580121 | FTHL8 | Homo sapiens ferritin, heavy polypeptide-like | NR_002203.1 | 23286 | 4.64E−06 | 1 |
| | | | 8 (FTHL8) on chromosome X | | | | |
| 226 | 240600 | LOC389599 | PREDICTED: Homo sapiens similar to | XM_372002.3 | 23285 | 4.48E−04 | 1 |
| | | | amyotrophic lateral sclerosis 2 (juvenile) | | | | |
| | | | chromosome region, candidate 2 | | | | |
| | | | (LOC389599), mRNA | | | | |
| 227 | 5360102 | C20ORF108 | Homo sapiens family with sequence similarity | NM_080821.2 | 22984 | 1.84E−03 | 1 |
| | | | 210, member B (FAM210B), mRNA | | | | |
| 228 | 7160608 | SIAH2 | Homo sapiens siah E3 ubiquitin protein ligase | NM_005067.5 | 22682 | 4.31E−05 | 1 |
| | | | 2 (SIAH2), mRNA | | | | |
| 229 | 1450523 | LRRK2 | PREDICTED: Homo sapiens leucine-rich | XM_930820.1 | 22676 | 1.31E−14 | 1 |
| | | | repeat kinase 2, transcript variant 2 (LRRK2), | | | | |
| | | | mRNA | | | | |
| 230 | 5570484 | HP | Homo sapiens haptoglobin (HP), transcript | NM_005143.3 | 22645 | 1.09E−05 | 1 |
| | | | variant 1, mRNA | | | | |
| 231 | 1770678 | IL4R | #NV | #NV | 22625 | 2.73E−07 | 1 |
| 232 | 7200367 | GNG11 | Homo sapiens guanine nucleotide binding | NM_004126.3 | 22249 | 7.11E−11 | 1 |
| | | | protein (G protein), gamma 11 (GNG11), | | | | |
| | | | mRNA | | | | |
| 233 | 5220477 | IFI27 | Homo sapiens interferon, alpha-inducible | NM_005532.3 | 22117 | 0.029515916 | 1 |
| | | | protein 27 (IFI27), transcript variant 2, mRNA | | | | |
| 234 | 670041 | HS.554324 | full-length cDNA clone CS0DI056YK21 of | CR596519.1 | 22072 | 3.25E−17 | −1 |
| | | | Placenta Cot 25-normalized of Homo sapiens | | | | |
| | | | (human) | | | | |
| 235 | 4210128 | HS.389491 | AW020492 df10f04.y1 Morton Fetal Cochlea | AW020492.2 | 21754 | 9.04E−12 | 1 |
| | | | Homo sapiens cDNA clone IMAGE: 2483071 | | | | |
| | | | 5′, mRNA sequence | | | | |
| 236 | 3180437 | GLRX5 | Homo sapiens glutaredoxin 5 (GLRX5), | NM_016417.2 | 21556 | 2.31E−07 | 1 |
| | | | nuclear gene encoding mitochondrial protein, | | | | |
| | | | mRNA | | | | |
| 237 | 6020196 | GYPB | Homo sapiens glycophorin B (MNS blood | NM_002100.4 | 21346 | 1.54E+00 | 1 |
| | | | group) (GYPB), mRNA | | | | |
| 238 | 3310091 | DEFA3 | Homo sapiens defensin, alpha 3, neutrophil- | NM_005217.3 | 21222 | 7.62E−03 | 1 |
| | | | specific (DEFA3), mRNA | | | | |
| 239 | 2070341 | LOC643313 | PREDICTED: Homo sapiens similar to | XM_933030.1 | 21216 | 1.03E−10 | 1 |
| | | | hypothetical protein LOC284701, transcript | | | | |
| | | | variant 1 (LOC643313), mRNA | | | | |
| 240 | 7320411 | ZDHHC19 | Homo sapiens zinc finger, DHHC-type | NM_144637.2 | 21099 | 0.018970076 | 1 |
| | | | containing 19 (ZDHHC19), mRNA | | | | |
| 241 | 4290692 | CAMK2A | Homo sapiens calcium/calmodulin-dependent | NM_171825.2 | 21066 | 0.014443988 | −1 |
| | | | protein kinase II alpha (CAMK2A), transcript | | | | |
| | | | variant 2, mRNA | | | | |
| 242 | 5290070 | LOC641848 | PREDICTED: Homo sapiens similar to | XM_935588.1 | 21058 | 0.000724407 | 1 |
| | | | ribosomal protein S3a (LOC641848), mRNA | | | | |
| 243 | 6760255 | CYP1B1 | Homo sapiens cytochrome P450, family 1, | NM_000104.3 | 21015 | 2.33E−06 | 1 |
| | | | subfamily B, polypeptide 1 (CYP1B1), mRNA | | | | |
| 244 | 7560072 | PRMT2 | Homo sapiens protein arginine | NM_206962.2 | 20610 | 1.12E−09 | 1 |
| | | | methyltransferase 2 (PRMT2), transcript | | | | |
| | | | variant 1, mRNA | | | | |
| 245 | 6110075 | LOC653778 | PREDICTED: Homo sapiens similar to solute | XM_929667.1 | 20518 | 4.95E−15 | 1 |
| | | | carrier family 25, member 37 (LOC653778), | | | | |
| | | | mRNA | | | | |
| 246 | 4180369 | RIS1 | Homo sapiens transmembrane protein 158 | NM_015444.2 | 20481 | 5.28E−02 | 1 |
| | | | (gene/pseudogene) (TMEM158), mRNA | | | | |
| 247 | 20070 | HS.520591 | AW273831 xv24e03.x1 | AW273831.1 | 20343 | 8.93E−08 | 1 |
| | | | Soares_NFL_T_GBC_S1 Homo sapiens | | | | |
| | | | cDNA clone IMAGE: 2814076 3′, mRNA | | | | |
| | | | sequence | | | | |
| 248 | 5290259 | AZU1 | Homo sapiens azurocidin 1 (AZU1), mRNA | NM_001700.3 | 20124 | 0.000185301 | 1 |
| 249 | 5720450 | MPO | Homo sapiens myeloperoxidase (MPO), | NM_000250.1 | 19940 | 1.25E+00 | 1 |
| | | | nuclear gene encoding mitochondrial protein, | | | | |
| | | | mRNA | | | | |
| 250 | 7040224 | TRIM58 | Homo sapiens tripartite motif containing 58 | NM_015431.3 | 19766 | 1.12E−01 | 1 |
| | | | (TRIM58), mRNA | | | | |
| 251 | 6510202 | CLIC3 | Homo sapiens chloride intracellular channel 3 | NM_004669.2 | 19657 | 3.19E−10 | −1 |
| | | | (CLIC3), mRNA | | | | |
| 252 | 6100176 | IL1R2 | Homo sapiens interleukin 1 receptor, type II | NM_173343.1 | 19587 | 0.000151711 | 1 |
| | | | (IL1R2), transcript variant 2, mRNA | | | | |
| 253 | 3420367 | RPL27A | Homo sapiens ribosomal protein L27a | NM_000990.4 | 19466 | 1.34E−03 | 1 |
| | | | (RPL27A), mRNA | | | | |
| 254 | 7330093 | HLA-DRB1 | Homo sapiens major histocompatibility | NM_002124.3 | 19396 | 0.000574076 | −1 |
| | | | complex, class II, DR beta 1 (HLA-DRB1), | | | | |
| | | | transcript variant 1, mRNA | | | | |
| 255 | 730129 | PTMA | Homo sapiens prothymosin, alpha (PTMA), | NM_002823.4 | 18892 | 8.72E−03 | 1 |
| | | | transcript variant 2, mRNA | | | | |
| 256 | 6280113 | CD164 | Homo sapiens CD164 molecule, sialomucin | NM_006016.4 | 18779 | 3.00E−07 | 1 |
| | | | (CD164), transcript variant 1, mRNA | | | | |
| 257 | 380731 | TUBA4A | Homo sapiens tubulin, alpha 4a (TUBA4A), | NM_006000.1 | 18620 | 2.89E−11 | 1 |
| | | | mRNA | | | | |
| 258 | 650504 | CHPT1 | Homo sapiens choline phosphotransferase 1 | NM_020244.2 | 18506 | 2.00E−06 | 1 |
| | | | (CHPT1), mRNA | | | | |
| 259 | 5700168 | RIOK3 | Homo sapiens RIO kinase 3 (yeast) (RIOK3), | NM_003831.3 | 18431 | 4.19E−03 | 1 |
| | | | mRNA | | | | |
| 260 | 290279 | FCRLA | Homo sapiens Fc receptor-like A (FCRLA), | NM_032738.3 | 18363 | 4.92E−07 | −1 |
| | | | transcript variant 2, mRNA | | | | |
| 261 | 6980168 | LOC641704 | PREDICTED: Homo sapiens similar to | XM_294802.5 | 18297 | 1.24E−07 | 1 |
| | | | hypothetical protein LOC284701, transcript | | | | |
| | | | variant 1 (LOC641704), mRNA | | | | |
| 262 | 290743 | GNLY | Homo sapiens granulysin (GNLY), transcript | NM_006433.3 | 18173 | 7.72E−05 | −1 |
| | | | variant NKG5, mRNA | | | | |
| 263 | 1820110 | SESN3 | Homo sapiens sestrin 3 (SESN3), mRNA | NM_144665.2 | 18158 | 1.33E−05 | 1 |
| 264 | 3440377 | PKN2 | Homo sapiens protein kinase N2 (PKN2), | NM_006256.2 | 18124 | 1.27E−14 | 1 |
| | | | mRNA | | | | |
| 265 | 4890095 | RPL7 | Homo sapiens ribosomal protein L7 (RPL7), | NM_000971.3 | 17949 | 6.35E−03 | 1 |
| | | | mRNA | | | | |
| 266 | 6560114 | RPLP1 | Homo sapiens ribosomal protein, large, P1 | NM_001003.2 | 17892 | 1.36E−01 | 1 |
| | | | (RPLP1), transcript variant 1, mRNA | | | | |
| 267 | 4850192 | CD6 | Homo sapiens CD6 molecule (CD6), transcript | NM_006725.4 | 17833 | 2.95E−13 | −1 |
| | | | variant 1, mRNA | | | | |
| 268 | 1010504 | LOC646463 | PREDICTED: Homo sapiens similar to | XM_929387.2 | 17621 | 2.31E−05 | 1 |
| | | | Ubiquitin-conjugating enzyme E2 H (Ubiquitin- | | | | |
| | | | protein ligase H) (Ubiquitin carrier protein H) | | | | |
| | | | (UBCH2) (E2-20K) (LOC646463), mRNA | | | | |
| 269 | 4260576 | LOC649682 | PREDICTED: Homo sapiens similar to | XM_938755.2 | 17519 | 4.59E+00 | 1 |
| | | | ribosomal protein L31 (LOC653773), mRNA | | | | |
| 270 | 3440392 | FLJ20273 | Homo sapiens RNA binding motif protein 47 | NM_019027.3 | 17362 | 1.13E−13 | 1 |
| | | | (RBM47), transcript variant 2, mRNA | | | | |
| 271 | 7560653 | ALAS2 | Homo sapiens aminolevulinate, delta-, | NM_000032.4 | 16729 | 0.000282052 | 1 |
| | | | synthase 2 (ALAS2), nuclear gene encoding | | | | |
| | | | mitochondrial protein, transcript variant 1, | | | | |
| | | | mRNA | | | | |
| 272 | 6450672 | IGJ | Homo sapiens immunoglobulin J polypeptide, | NM_144646.3 | 16669 | 1.46E+00 | 1 |
| | | | linker protein for immunoglobulin alpha and | | | | |
| | | | mu polypeptides (IGJ), mRNA | | | | |
| 273 | 6940348 | BPGM | Homo sapiens 2,3-bisphosphoglycerate | NM_001724.4 | 16603 | 0.001070662 | 1 |
| | | | mutase (BPGM), transcript variant 1, mRNA | | | | |
| 274 | 3940446 | EVI2A | Homo sapiens ecotropic viral integration site | NM_014210.3 | 16601 | 1.05E−04 | 1 |
| | | | 2A (EVI2A), transcript variant 2, mRNA | | | | |
| 275 | 1170390 | STOM | Homo sapiens stomatin (STOM), transcript | NM_004099.4 | 16272 | 4.77E−09 | 1 |
| | | | variant 1, mRNA | | | | |
| 276 | 4220273 | LOC387753 | PREDICTED: Homo sapiens similar to 60S | XM_370611.5 | 16186 | 1.46E−05 | 1 |
| | | | ribosomal protein L21 (LOC387753), mRNA | | | | |
| 277 | 6550709 | LOC440732 | PREDICTED: Homo sapiens similar to 40S | XM_496441.2 | 16105 | 2.21E−01 | 1 |
| | | | ribosomal protein S7 (S8) (LOC440732), | | | | |
| | | | mRNA | | | | |
| 278 | 4570474 | EPB49 | Homo sapiens erythrocyte membrane protein | NM_001978.2 | 16094 | 1.84E−03 | 1 |
| | | | band 4.9 (dematin) (EPB49), transcript variant | | | | |
| | | | 1, mRNA | | | | |
| 279 | 7650356 | RHOQ | Homo sapiens ras homolog family member Q | NM_012249.3 | 16065 | 1.58E−13 | 1 |
| | | | (RHOQ), mRNA | | | | |
| 280 | 1820491 | PIK3AP1 | Homo sapiens phosphoinositide-3-kinase | NM_152309.2 | 15738 | 1.72E−06 | 1 |
| | | | adaptor protein 1 (PIK3AP1), mRNA | | | | |
| 281 | 770333 | UQCRH | Homo sapiens ubiquinol-cytochrome c | NM_006004.2 | 15719 | 3.60E−06 | 1 |
| | | | reductase hinge protein (UQCRH), nuclear | | | | |
| | | | gene encoding mitochondrial protein, mRNA | | | | |
| 282 | 7200593 | IGF2BP2 | Homo sapiens insulin-like growth factor 2 | NM_006548.4 | 15658 | 2.77E−05 | 1 |
| | | | mRNA binding protein 2 (IGF2BP2), transcript | | | | |
| | | | variant 1, mRNA | | | | |
| 283 | 60091 | C1ORF63 | Homo sapiens chromosome 1 open reading | NM_020317.3 | 15588 | 1.68E−11 | 1 |
| | | | frame 63 (C1orf63), mRNA | | | | |
| 284 | 2690068 | PBEF1 | Homo sapiens pre-B-cell colony enhancing | NM_182790.1 | 15244 | 2.16E−03 | 1 |
| | | | factor 1 (PBEF1), transcript variant 2, mRNA | | | | |
| 285 | 3460477 | RETN | Homo sapiens resistin (RETN), transcript | NM_020415.3 | 15193 | 7.83E+00 | 1 |
| | | | variant 1, mRNA | | | | |
| 286 | 2510133 | SAP30 | Homo sapiens Sin3A-associated protein, | NM_003864.3 | 15126 | 2.42E−07 | 1 |
| | | | 30 kDa (SAP30), mRNA | | | | |
| 287 | 6330133 | LOC648294 | PREDICTED: Homo sapiens similar to 60S | XM_939952.1 | 15002 | 4.50E−05 | 1 |
| | | | ribosomal protein L23a (LOC648294), mRNA | | | | |
| 288 | 6330010 | MCEMP1 | Homo sapiens chromosome 19 open reading | NM_174918.2 | 14885 | 3.24E−01 | 1 |
| | | | frame 59 (C19orf59), mRNA | | | | |
| 289 | 4760767 | ZNF223 | Homo sapiens zinc finger protein 223 | NM_013361.4 | 14704 | 3.52E−06 | 1 |
| | | | (ZNF223), mRNA | | | | |
| 290 | 290360 | UBE2H | Homo sapiens ubiquitin-conjugating enzyme | NM_003344.3 | 14688 | 8.13E−07 | 1 |
| | | | E2H (UBE2H), transcript variant 1, mRNA | | | | |
| 291 | 2060347 | EVL | Homo sapiens Enah/Vasp-like (EVL), mRNA | NM_016337.2 | 14679 | 3.69E−13 | −1 |
| 292 | 2490056 | LOC644972 | PREDICTED: Homo sapiens similar to 40S | XR_001449.2 | 14597 | 1.37E−01 | 1 |
| | | | ribosomal protein S3a (V-fos transformation | | | | |
| | | | effector protein) (LOC644972), mRNA | | | | |
| 293 | 6330221 | GNL3L | Homo sapiens guanine nucleotide binding | NM_019067.5 | 14546 | 2.47E−07 | 1 |
| | | | protein-like 3 (nucleolar)-like (GNL3L), | | | | |
| | | | transcript variant 2, mRNA | | | | |
| 294 | 6370307 | C14ORF45 | Homo sapiens chromosome 14 open reading | NM_025057.2 | 14538 | 4.11E−04 | 1 |
| | | | frame 45 (C14orf45), mRNA | | | | |
| 295 | 6590520 | CAPZA1 | Homo sapiens capping protein (actin filament) | NM_006135.2 | 14518 | 8.55E−01 | 1 |
| | | | muscle Z-line, alpha 1 (CAPZA1), mRNA | | | | |
| 296 | 2900463 | GNLY | Homo sapiens granulysin (GNLY), transcript | NM_012483.2 | 14444 | 1.28E−04 | −1 |
| | | | variant 519, mRNA | | | | |
| 297 | 6620575 | LOC644162 | PREDICTED: Homo sapiens similar to septin | XM_933956.1 | 14400 | 3.60E−07 | 1 |
| | | | 7, transcript variant 4 (LOC644162), mRNA | | | | |
| 298 | 5890019 | WASPIP | Homo sapiens WAS/WASL interacting protein | NM_003387.4 | 14354 | 7.57E−05 | 1 |
| | | | family, member 1 (WIPF1), transcript variant | | | | |
| | | | 1, mRNA | | | | |
| 299 | 7200255 | IFI44L | Homo sapiens interferon-induced protein 44- | NM_006820.2 | 14310 | 0.003680493 | 1 |
| | | | like (IFI44L), mRNA | | | | |
| 300 | 6450747 | LOC441155 | PREDICTED: Homo sapiens similar to Zinc | XM_930970.1 | 13804 | 3.04E−07 | 1 |
| | | | finger CCCH-type domain containing protein | | | | |
| | | | 11A, transcript variant 3 (LOC441155), mRNA | | | | |
| 301 | 6770075 | JAZF1 | Homo sapiens JAZF zinc finger 1 (JAZF1), | NM_175061.3 | 13791 | 6.13E−07 | 1 |
| | | | mRNA | | | | |
| 302 | 4730114 | MYL9 | Homo sapiens myosin, light chain 9, | NM_006097.4 | 13629 | 4.47E−08 | 1 |
| | | | regulatory (MYL9), transcript variant 1, mRNA | | | | |
| 303 | 7210497 | GP1BB | Homo sapiens glycoprotein Ib (platelet), beta | NM_000407.4 | 13573 | 1.96E−07 | 1 |
| | | | polypeptide (GP1BB), mRNA | | | | |
| 304 | 1510523 | PTGES3 | Homo sapiens prostaglandin E synthase 3 | NM_006601.5 | 13534 | 8.34E−12 | 1 |
| | | | (cytosolic) (PTGES3), mRNA | | | | |
| 305 | 3460224 | SLC1A5 | Homo sapiens solute carrier family 1 (neutral | NM_005628.2 | 13395 | 3.77E−03 | 1 |
| | | | amino acid transporter), member 5 (SLC1A5), | | | | |
| | | | transcript variant 1, mRNA | | | | |
| 306 | 6290561 | HLA-DQA1 | PREDICTED: Homo sapiens major | XM_936120.1 | 13211 | 1.55E−05 | −1 |
| | | | histocompatibility complex, class II, DQ alpha | | | | |
| | | | 1, transcript variant 2 (HLA-DQA1), mRNA | | | | |
| 307 | 5890184 | LOC284230 | PREDICTED: Homo sapiens similar to | XM_208185.7 | 13146 | 0.000724095 | 1 |
| | | | mCG7611 (LOC284230), mRNA | | | | |
| 308 | 2600632 | FLJ40722 | PREDICTED: Homo sapiens hypothetical | XM_942096.1 | 13123 | 2.14E−07 | 1 |
| | | | protein FLJ40722, transcript variant 3 | | | | |
| | | | (FLJ40722), mRNA | | | | |
| 309 | 3610296 | NFIC | Homo sapiens nuclear factor I/C (CCAAT- | NM_005597.3 | 13093 | 9.70E−09 | 1 |
| | | | binding transcription factor) (NFIC), transcript | | | | |
| | | | variant 5, mRNA | | | | |
| 310 | 7650025 | DSC2 | Homo sapiens desmocollin 2 (DSC2), | NM_004949.3 | 13074 | 4.58E−04 | 1 |
| | | | transcript variant Dsc2b, mRNA | | | | |
| 311 | 1580450 | LOC643870 | PREDICTED: Homo sapiens similar to | XM_927140.1 | 12973 | 5.83E−03 | 1 |
| | | | Translationally-controlled tumor protein | | | | |
| | | | (TCTP) (p23) (Histamine-releasing factor) | | | | |
| | | | (HRF) (Fortilin) (LOC643870), mRNA | | | | |
| 312 | 1110575 | ABLIM1 | Homo sapiens actin binding LIM protein 1 | NM_006720.3 | 12875 | 3.09E−16 | −1 |
| | | | (ABLIM1), transcript variant 4, mRNA | | | | |
| 313 | 4920408 | LOC644914 | PREDICTED: Homo sapiens similar to H3 | XM_930111.2 | 12748 | 6.43E−12 | 1 |
| | | | histone, family 3B (LOC644914), mRNA | | | | |
| 314 | 4200685 | MYOM2 | Homo sapiens myomesin (M-protein) 2, | NM_003970.2 | 12676 | 0.002711113 | −1 |
| | | | 165 kDa (MYOM2), mRNA | | | | |
| 315 | 840072 | HS.541992 | BG055310 nad45e06.x1 NCI_CGAP_Lu24 | BG055310.1 | 12509 | 1.43E−03 | 1 |
| | | | Homo sapiens cDNA clone IMAGE: 3368531 | | | | |
| | | | 3′, mRNA sequence | | | | |
| 316 | 6590730 | TPM3 | Homo sapiens tropomyosin 3 (TPM3), | NM_153649.3 | 12447 | 2.97E−08 | 1 |
| | | | transcript variant 2, mRNA | | | | |
| 317 | 7330377 | KPNA2 | Homo sapiens karyopherin alpha 2 (RAG | NM_002266.2 | 12392 | 3.37E−13 | 1 |
| | | | cohort 1, importin alpha 1) (KPNA2), mRNA | | | | |
| 318 | 1780270 | EIF1AY | Homo sapiens eukaryotic translation initiation | NM_004681.2 | 12367 | 0.004142354 | 1 |
| | | | factor 1A, Y-linked (EIF1AY), mRNA | | | | |
| 319 | 4150224 | MMP9 | Homo sapiens matrix metallopeptidase 9 | NM_004994.2 | 12317 | 2.89E−01 | 1 |
| | | | (gelatinase B, 92 kDa gelatinase, 92 kDa type | | | | |
| | | | IV collagenase) (MMP9), mRNA | | | | |
| 320 | 3830382 | RAXL1 | Homo sapiens retina and anterior neural fold | NM_032753.3 | 12151 | 3.41E−09 | 1 |
| | | | homeobox 2 (RAX2), mRNA | | | | |
| 321 | 1230358 | OLR1 | Homo sapiens oxidized low density lipoprotein | NM_002543.3 | 12146 | 9.91E−03 | 1 |
| | | | (lectin-like) receptor 1 (OLR1), transcript | | | | |
| | | | variant 1, mRNA | | | | |
| 322 | 5220026 | IFNAR2 | Homo sapiens interferon (alpha, beta and | NM_207585.1 | 12042 | 1.68E−08 | 1 |
| | | | omega) receptor 2 (IFNAR2), transcript variant | | | | |
| | | | 1, mRNA | | | | |
| 323 | 4050195 | HS.99472 | Homo sapiens genomic DNA; cDNA | AL080095.1 | 11930 | 1.88E−08 | 1 |
| | | | DKFZp564O0862 (from clone | | | | |
| | | | DKFZp564O0862) | | | | |
| 324 | 540491 | WSB2 | Homo sapiens WD repeat and SOCS box | NM_018639.3 | 11796 | 1.92E−16 | 1 |
| | | | containing 2 (WSB2), mRNA | | | | |
| 325 | 1780377 | LOC651919 | PREDICTED: Homo sapiens similar to Ras- | XM_941189.1 | 11747 | 8.84E−07 | 1 |
| | | | related C3 botulinum toxin substrate 1 (p21- | | | | |
| | | | Rac1) (LOC651919), mRNA | | | | |
| 326 | 5870221 | IFI44 | Homo sapiens interferon-induced protein 44 | NM_006417.4 | 11633 | 0.000570009 | 1 |
| | | | (IFI44), mRNA | | | | |
| 327 | 4050239 | EPB42 | Homo sapiens erythrocyte membrane protein | NM_000119.2 | 11581 | 2.86E+00 | 1 |
| | | | band 4.2 (EPB42), transcript variant 1, mRNA | | | | |
| 328 | 4900577 | LOC647100 | PREDICTED: Homo sapiens similar to 60S | XM_930115.1 | 11555 | 3.38E−03 | 1 |
| | | | ribosomal protein L38 (LOC647100), mRNA | | | | |
| 329 | 770309 | PLEK2 | Homo sapiens pleckstrin 2 (PLEK2), mRNA | NM_016445.1 | 11554 | 4.04E−04 | 1 |
| 330 | 2260148 | NELL2 | Homo sapiens NEL-like 2 (chicken) (NELL2), | NM_006159.2 | 11364 | 1.76E−11 | −1 |
| | | | transcript variant 2, mRNA | | | | |
| 331 | 6650215 | LYN | Homo sapiens v-yes-1 Yamaguchi sarcoma | NM_002350.3 | 11242 | 1.68E−10 | 1 |
| | | | viral related oncogene homolog (LYN), | | | | |
| | | | transcript variant 1, mRNA | | | | |
| 332 | 5890471 | NCR3 | Homo sapiens natural cytotoxicity triggering | NM_147130.2 | 11217 | 2.85E−12 | −1 |
| | | | receptor 3 (NCR3), transcript variant 1, mRNA | | | | |
| 333 | 3930138 | RAB33B | Homo sapiens RAB33B, member RAS | NM_031296.1 | 11201 | 8.50E−06 | 1 |
| | | | oncogene family (RAB33B), mRNA | | | | |
| 334 | 4210100 | MSL3L1 | Homo sapiens male-specific lethal 3 homolog | NM_006800.3 | 11148 | 7.06E−08 | 1 |
| | | | (Drosophila)(MSL3), transcript variant 3, | | | | |
| | | | mRNA | | | | |
| 335 | 2370121 | CCNY | Homo sapiens cyclin Y (CCNY), transcript | NM_145012.4 | 11123 | 3.79E−08 | 1 |
| | | | variant 1, mRNA | | | | |
| 336 | 160132 | CREB5 | Homo sapiens cAMP responsive element | NM_182898.2 | 11118 | 2.81E−11 | 1 |
| | | | binding protein 5 (CREB5), transcript variant | | | | |
| | | | 1, mRNA | | | | |
| 337 | 2190475 | HSD17B11 | Homo sapiens hydroxysteroid (17-beta) | NM_016245.3 | 11084 | 5.87E−06 | 1 |
| | | | dehydrogenase 11 (HSD17B11), mRNA | | | | |
| 338 | 10767 | SLCO3A1 | Homo sapiens solute carrier organic anion | NM_013272.3 | 10997 | 5.67E−01 | 1 |
| | | | transporter family, member 3A1 (SLCO3A1), | | | | |
| | | | transcript variant 1, mRNA | | | | |
| 339 | 4390093 | LOC440359 | PREDICTED: Homo sapiens similar to muscle | XM_496143.2 | 10983 | 2.13E−01 | 1 |
| | | | Y-box protein YB2 (LOC440359), mRNA | | | | |
| 340 | 5560079 | NLRP12 | Homo sapiens NLR family, pyrin domain | NM_033297.2 | 10889 | 2.59E−09 | 1 |
| | | | containing 12 (NLRP12), transcript variant 1, | | | | |
| | | | mRNA | | | | |
| 341 | 1780477 | TMOD1 | Homo sapiens tropomodulin 1 (TMOD1), | NM_003275.3 | 10795 | 0.000178732 | 1 |
| | | | transcript variant 1, mRNA | | | | |
| 342 | 1470669 | ANKRD33 | Homo sapiens ankyrin repeat domain 33 | NM_182608.3 | 10793 | 1.21E−10 | 1 |
| | | | (ANKRD33), transcript variant 2, mRNA | | | | |
| 343 | 1430762 | IRAK3 | Homo sapiens interleukin-1 receptor- | NM_007199.2 | 10730 | 8.65E−07 | 1 |
| | | | associated kinase 3 (IRAK3), transcript variant | | | | |
| | | | 1, mRNA | | | | |
| 344 | 3400470 | HS.407903 | Homo sapiens mRNA; cDNA | AL049435.1 | 10728 | 1.06E−03 | 1 |
| | | | DKFZp586B0220 (from clone | | | | |
| | | | DKFZp586B0220) | | | | |
| 345 | 1580010 | TRIP12 | Homo sapiens thyroid hormone receptor | NM_004238.1 | 10720 | 4.52E−18 | 1 |
| | | | interactor 12 (TRIP12), mRNA | | | | |
| 346 | 70722 | COX7B | Homo sapiens cytochrome c oxidase subunit | NM_001866.2 | 10720 | 4.05E−03 | 1 |
| | | | VIIb (COX7B), nuclear gene encoding | | | | |
| | | | mitochondrial protein, mRNA | | | | |
| 347 | 4040035 | TUBB1 | Homo sapiens tubulin, beta 1 class VI | NM_030773.3 | 10716 | 8.51E−06 | 1 |
| | | | (TUBB1), mRNA | | | | |
| 348 | 3850524 | CEP27 | Homo sapiens HAUS augmin-like complex, | NM_018097.2 | 10648 | 9.31E−06 | 1 |
| | | | subunit 2 (HAUS2), transcript variant 1, mRNA | | | | |
| 349 | 7400136 | HLA-DMA | Homo sapiens major histocompatibility | NM_006120.3 | 10617 | 5.27E−11 | −1 |
| | | | complex, class II, DM alpha (HLA-DMA), | | | | |
| | | | mRNA | | | | |
| 350 | 20575 | VTI1B | Homo sapiens vesicle transport through | NM_006370.2 | 10611 | 6.20E−03 | −1 |
| | | | interaction with t-SNAREs homolog 1B (yeast) | | | | |
| | | | (VTI1B), mRNA | | | | |
| 351 | 3460661 | DCTN4 | Homo sapiens dynactin 4 (p62) (DCTN4), | NM_016221.3 | 10552 | 4.34E−15 | 1 |
| | | | transcript variant 2, mRNA | | | | |
| 352 | 7000133 | BCL11B | Homo sapiens B-cell CLL/lymphoma 11B (zinc | NM_022898.1 | 10542 | 8.25E−16 | −1 |
| | | | finger protein) (BCL11B), transcript variant 2, | | | | |
| | | | mRNA | | | | |
| 353 | 1690162 | LOC642115 | PREDICTED: Homo sapiens similar to | XM_936258.2 | 10484 | 2.77E−06 | 1 |
| | | | ribosomal protein S8 (LOC642115), mRNA | | | | |
| 354 | 1170400 | C12ORF57 | Homo sapiens chromosome 12 open reading | NM_138425.2 | 10412 | 3.52E−07 | −1 |
| | | | frame 57 (C12orf57), mRNA | | | | |
| 355 | 1190274 | RPL18 | Homo sapiens ribosomal protein L18 (RPL18), | NM_000979.2 | 10396 | 1.46E−09 | −1 |
| | | | mRNA | | | | |
| 356 | 1450184 | C5ORF41 | Homo sapiens CREB3 regulatory factor | NM_153607.2 | 10394 | 7.85E−09 | 1 |
| | | | (CREBRF), transcript variant 1, mRNA | | | | |
| 357 | 1500634 | USF1 | Homo sapiens , upstream transcription factor 1 | NM_007122.3 | 10382 | 1.59E−05 | −1 |
| | | | (USF1), transcript variant 1, mRNA | | | | |
| 358 | 6560274 | VIL2 | Homo sapiens ezrin (EZR), transcript variant | NM_003379.4 | 10378 | 1.26E−11 | −1 |
| | | | 1, mRNA | | | | |
| 359 | 1110670 | LOC647908 | PREDICTED: Homo sapiens similar to RAS | XM_938419.1 | 10371 | 9.02E−06 | 1 |
| | | | related protein 1b isoform 1 (LOC647908), | | | | |
| | | | mRNA | | | | |
| 360 | 60482 | IFI16 | Homo sapiens interferon, gamma-inducible | NM_005531.2 | 10336 | 3.90E−13 | 1 |
| | | | protein 16 (IFI16), transcript variant 2, mRNA | | | | |
| 361 | 6350372 | LOC643287 | PREDICTED: Homo sapiens similar to | XM_928075.2 | 10327 | 2.30E−06 | 1 |
| | | | prothymosin alpha, transcript variant 1 | | | | |
| | | | (LOC643287), mRNA | | | | |

TABLE 4

| Probe ID | GeneSymbol |
|---|---|
| 6200563 | ZNF654 |
| 430382 | UBE2G1 |
| 5900156 | TUBA1B |
| 2370524 | TNFAIP6 |
| 5720681 | TIPARP |
| 3460189 | STXBP5 |
| 2100035 | STK17B |
| 3460674 | SRPK1 |
| 5090477 | PIP4K2B |
| 2000390 | PFTK1 |
| 270717 | PELI2 |
| 3780689 | NT5C3 |
| 7200681 | NA |
| 5490064 | NA |
| 4060138 | NA |
| 3830390 | NA |
| 7150634 | MY09A |
| 6550520 | LYSMD2 |
| 3830341 | LYRM1 |
| 2570703 | LOC400566 |
| 1820598 | ICAM2 |
| 610563 | HMGB2 |
| 4730195 | HIST1H4H |
| 5860400 | HIST1H2AE |
| 6180427 | GPR160 |
| 3610504 | GNE |
| 520332 | GALT |
| 5860500 | EIF2C3 |
| 4280332 | DDX24 |
| 3140039 | CYB5R4 |
| 5290452 | CPEB3 |

TABLE 5

| GeneSymbol | probe set | F statistic | p-value |
|---|---|---|---|
| | 1820255 | 14.1185 | 5.49E−06 |
| | 4050195 | 10.1948 | 0.000112435 |
| | 5860196 | 9.90952 | 0.000141309 |
| | 4050270 | 9.3253 | 0.000226572 |
| | 4260102 | 9.07091 | 0.000278766 |
| | 4920142 | 8.54205 | 0.000430427 |
| | 670041 | 8.52692 | 0.000435839 |
| | 6980274 | 8.47291 | 0.000455735 |
| | 3990639 | 8.45296 | 0.000463322 |
| | 4730577 | 8.18449 | 0.000578904 |
| | 2450497 | 7.93996 | 0.000709867 |
| | 1690504 | 7.77255 | 0.000816714 |
| | 4280240 | 7.66009 | 0.00089762 |
| | 5860682 | 7.64396 | 0.000909879 |
| | 4060017 | 7.47457 | 0.0010495 |
| | 1660451 | 7.38896 | 0.00112822 |
| | 5560093 | 7.34668 | 0.00116931 |
| | 3940020 | 7.34348 | 0.00117248 |
| | 6550333 | 7.21047 | 0.00131242 |
| | 1570703 | 7.15098 | 0.00138044 |
| | 4150402 | 7.12535 | 0.00141086 |
| | 1110358 | 7.05133 | 0.00150259 |
| | 4010296 | 6.91861 | 0.00168268 |
| | 2450343 | 6.84874 | 0.00178623 |
| | 2850762 | 6.84117 | 0.00179784 |
| | 4070280 | 6.77187 | 0.00190772 |
| | 650343 | 6.6981 | 0.00203228 |
| | 1990113 | 6.67761 | 0.00206835 |
| | 5130154 | 6.62206 | 0.00216943 |
| | 6840408 | 6.58787 | 0.00223416 |
| | 5390187 | 6.58213 | 0.00224521 |
| | 6940246 | 6.53962 | 0.00232886 |
| | 5270450 | 6.48382 | 0.00244355 |
| | 5420148 | 6.47773 | 0.00245641 |
| | 3930632 | 6.40425 | 0.0026172 |
| | 3940719 | 6.3459 | 0.00275252 |
| | 1940341 | 6.33404 | 0.00278088 |
| | 4610129 | 6.32188 | 0.00281029 |
| | 2060220 | 6.30442 | 0.00285305 |
| | 3450187 | 6.28972 | 0.00288959 |
| | 6060400 | 6.21906 | 0.00307198 |
| | 4050692 | 6.19827 | 0.00312787 |
| | 4560463 | 6.19294 | 0.00314233 |
| | 540390 | 6.18482 | 0.00316455 |
| | 650014 | 6.15899 | 0.00323628 |
| | 4010025 | 6.15102 | 0.00325875 |
| | 6860743 | 6.12387 | 0.00333651 |
| | 4730088 | 6.0908 | 0.00343378 |
| | 5570632 | 6.07838 | 0.00347105 |
| | 4880373 | 6.05754 | 0.00353452 |
| | 2120356 | 6.04802 | 0.00356394 |
| | 240653 | 5.98976 | 0.00374938 |
| | 2100482 | 5.9589 | 0.00385156 |
| | 6220706 | 5.95393 | 0.00386829 |
| | 2000474 | 5.95307 | 0.00387119 |
| | 6040326 | 5.90341 | 0.00404257 |
| | 5360605 | 5.89899 | 0.00405818 |
| | 6650482 | 5.89178 | 0.00408381 |
| | 6510452 | 5.87022 | 0.00416141 |
| | 5130747 | 5.80679 | 0.00439863 |
| | 4900471 | 5.79219 | 0.00445516 |
| | 1500433 | 5.78948 | 0.00446577 |
| | 6660056 | 5.74704 | 0.00463477 |
| | 4560543 | 5.72865 | 0.00471003 |
| | 3520040 | 5.71127 | 0.0047823 |
| | 7210719 | 5.71103 | 0.00478332 |
| | 1410678 | 5.70918 | 0.00479109 |
| | 4900441 | 5.67343 | 0.00494362 |
| | 5130553 | 5.67112 | 0.00495368 |
| | 5960307 | 5.65178 | 0.00503844 |
| | 2510324 | 5.64786 | 0.0050558 |
| | 3800524 | 5.64519 | 0.00506767 |
| | 4560451 | 5.6406 | 0.00508814 |
| | 4200148 | 5.62095 | 0.00517666 |
| | 5050112 | 5.60435 | 0.00525268 |
| | 5810521 | 5.60106 | 0.0052679 |
| | 3390333 | 5.60078 | 0.0052692 |
| | 5560736 | 5.59779 | 0.00528302 |
| | 2810537 | 5.57818 | 0.00537487 |
| | 3180750 | 5.56903 | 0.00541829 |
| | 1070131 | 5.55883 | 0.00546706 |
| | 4670195 | 5.55044 | 0.00550756 |
| | 3370646 | 5.5398 | 0.00555938 |
| | 4780386 | 5.53607 | 0.00557761 |
| | 7650553 | 5.52949 | 0.00561002 |
| | 650431 | 5.52747 | 6.00562002 |
| | 3850020 | 5.49933 | 0.00576095 |
| | 6650576 | 5.48985 | 0.0058092 |
| | 3800192 | 5.48005 | 0.00585956 |
| | 520154 | 5.47519 | 0.0058847 |
| | 2060026 | 5.46904 | 0.00591666 |
| | 4220367 | 5.46097 | 0.00595889 |
| | 5550270 | 5.45728 | 0.00597832 |
| | 4810327 | 5.45721 | 0.0059787 |
| | 5340338 | 5.45264 | 0.00600282 |
| | 6420370 | 5.44511 | 0.0060428 |
| | 4220523 | 5.41643 | 0.00619754 |
| | 5670735 | 5.40263 | 0.00627348 |
| | 4540088 | 5.39708 | 0.00630423 |
| | 7570725 | 5.38871 | 0.00635101 |
| | 1050128 | 5.38517 | 0.00637087 |
| | 6420541 | 5.35224 | 0.0065588 |
| | 3890491 | 5.31733 | 0.00676428 |
| | 4220450 | 5.30399 | 0.00684454 |
| | 2450424 | 5.30056 | 0.00686532 |
| | 2190451 | 5.29585 | 0.00689401 |
| | 5550634 | 5.29177 | 0.00691888 |
| | 150711 | 5.27243 | 0.00703831 |
| | 3850367 | 5.26624 | 0.00707693 |
| | 510653 | 5.22629 | 0.00733174 |
| | 2640441 | 5.22571 | 0.00733549 |
| | 630372 | 5.21527 | 0.00740365 |
| | 840139 | 5.2066 | 0.00746074 |
| | 830482 | 5.1925 | 0.00755456 |
| | 7320041 | 5.18344 | 0.00761544 |
| | 1570424 | 5.17193 | 0.0076936 |
| | 830167 | 5.14976 | 0.00784637 |
| | 2650520 | 5.1314 | 0.00797525 |
| | 5130204 | 5.13014 | 0.0079842 |
| | 5090647 | 5.12576 | 0.00801528 |
| | 4900064 | 5.11547 | 0.00808884 |
| | 4900497 | 5.11362 | 0.0081021 |
| | 6560292 | 5.10836 | 0.00814009 |
| | 5670445 | 5.07695 | 0.00837036 |
| | 50242 | 5.0629 | 0.00847556 |
| | 2650243 | 5.04917 | 0.00857968 |
| | 4490612 | 5.03315 | 0.00870279 |
| | 2710291 | 5.01493 | 0.00884494 |
| | 6370592 | 5.00692 | 0.00890826 |
| | 6400047 | 4.99624 | 0.00899332 |
| | 6280682 | 4.97761 | 0.00914377 |
| | 5420487 | 4.97558 | 0.00916028 |
| AAAS | 870088 | 5.22423 | 0.00734515 |
| AARS | 2490747 | 5.42843 | 0.00613228 |
| ABCA1 | 6110088 | 8.32635 | 0.000514553 |
| ABCC5 | 7610097 | 5.30928 | 0.00681258 |
| ABLIM1 | 1110575 | 6.03632 | 0.00360039 |
| ACO2 | 10068 | 5.1771 | 0.0076584 |
| ACOT1 | 7510224 | 5.55523 | 0.00548442 |
| ACOX1 | 4010048 | 8.48072 | 0.000452803 |
| ACSS1 | 5090047 | 6.15103 | 0.00325872 |
| ADA | 3400328 | 5.2374 | 0.00725998 |
| ADAR | 1410358 | 6.02935 | 0.00362232 |
| ADCK2 | 2630524 | 5.25902 | 0.00712234 |
| ADCY4 | 4230653 | 6.54626 | 0.00231559 |
| AGTPBP1 | 4860132 | 5.10886 | 0.00813646 |
| AHSA1 | 3990192 | 6.40918 | 0.00260606 |
| AKR1A1 | 1300768 | 6.29116 | 0.00288598 |
| AKR1B1 | 5890327 | 6.36525 | 0.00270686 |
| ALB | 2710427 | 6.2086 | 0.00309997 |
| ALDH16A1 | 4050411 | 5.68965 | 0.0048738 |
| ALDH8A1 | 2340358 | 5.11504 | 0.0080919 |
| ALG8 | 4810431 | 5.57112 | 0.00540833 |
| ALG9 | 2120681 | 6.61337 | 0.0021857 |
| ALKBH5 | 2100221 | 5.93864 | 0.0039202 |
| ANAPC5 | 7380288 | 8.15472 | 0.000593428 |
| ANKDD1A | 3290296 | 8.43338 | 0.000470888 |
| ANKHD1 | 540338 | 5.03817 | 0.00866398 |
| ANKK1 | 5910091 | 8.94699 | 0.000308507 |
| ANKMY1 | 4850541 | 6.47239 | 0.00246776 |
| ANKRD13 | 10543 | 10.3662 | 9.81E−05 |
| ANKRD17 | 2600097 | 5.89269 | 0.00408056 |
| ANKRD32 | 3120358 | 5.03412 | 0.00869527 |
| ANXA7 | 6770403 | 5.39807 | 0.00629876 |
| AP1S1 | 6270301 | 10.5654 | 8.37E−05 |
| AP1S1 | 2650075 | 9.66784 | 0.000171672 |
| AP1S1 | 7050072 | 5.5133 | 0.0056905 |
| AP3M1 | 6590278 | 10.2741 | 0.000105538 |
| APAF1 | 6350452 | 8.11784 | 0.000611935 |
| APEX1 | 1190647 | 5.35486 | 0.00654365 |
| APH1A | 2940224 | 8.65896 | 0.000390858 |
| APOBEC3F | 4070132 | 6.68502 | 0.00205524 |
| APOL3 | 5670274 | 5.63688 | 0.00510477 |
| APRT | 650358 | 5.28658 | 0.00695075 |
| AQP9 | 6770564 | 7.13272 | 0.00140204 |
| ARFIP1 | 6480647 | 5.60559 | 0.00524699 |
| ARHGAP17 | 1710100 | 5.18768 | 0.00758689 |
| ARHGAP21 | 3370487 | 5.5288 | 0.00561343 |
| ARID1A | 150148 | 5.28184 | 0.00697992 |
| ARID2 | 3850347 | 7.07364 | 0.00147432 |
| ARIH2 | 5390669 | 7.12379 | 0.00141273 |
| ARL6IP6 | 2710722 | 5.81679 | 0.00436035 |
| ARPC5 | 3930243 | 5.92342 | 0.00397259 |
| ARPC5L | 1770279 | 5.11671 | 0.00807995 |
| ARPP-19 | 2600008 | 6.84895 | 0.00178591 |
| ARPP-21 | 840762 | 5.70693 | 0.00480054 |
| ARRDC4 | 7040187 | 11.1023 | 5.48E−05 |
| ASB8 | 4280114 | 7.51678 | 0.00101277 |
| ASXL2 | 60750 | 5.09956 | 0.00820393 |
| ATIC | 6110768 | 9.0383 | 0.000286296 |
| ATP10B | 2630484 | 9.80095 | 0.000154203 |
| ATP11B | 540053 | 6.66493 | 0.00209099 |
| ATP1A1 | 1240440 | 5.42734 | 0.00613818 |
| ATP5A1 | 3130300 | 5.72996 | 0.00470464 |
| ATP5G2 | 6350360 | 5.25137 | 0.00717071 |
| ATP5I | 4570095 | 5.66902 | 0.00496281 |
| ATP7A | 1110259 | 5.74309 | 0.00465082 |
| ATXN1 | 3310470 | 6.06884 | 0.00349998 |
| B4GALT5 | 6980070 | 8.78232 | 0.000353132 |
| B4GALT7 | 6100220 | 6.51419 | 0.00238043 |
| BAZ1A | 4920204 | 6.08814 | 0.00344174 |
| BCL10 | 6290343 | 5.12073 | 0.00805115 |
| BCL11B | 7000133 | 7.56123 | 0.000975516 |
| BCL6 | 4640044 | 8.39922 | 0.000484396 |
| BIRC3 | 5390504 | 9.64575 | 0.000174761 |
| BLR1 | 1440291 | 5.74509 | 0.00464269 |
| BTBD10 | 4860296 | 6.23031 | 0.00304217 |
| BTN3A2 | 4920577 | 5.88592 | 0.00410478 |
| C10orf42 | 6480717 | 5.4839 | 0.00583975 |
| C10orf63 | 6580445 | 8.7534 | 0.000361627 |
| C11orf53 | 1240278 | 6.26539 | 0.00295111 |
| C12orf49 | 4280056 | 7.83599 | 0.000774407 |
| C14orf130 | 1690543 | 5.03059 | 0.00872262 |
| C14orf138 | 5360132 | 5.0372 | 0.00867146 |
| C14orf32 | 2750162 | 5.56789 | 0.00542371 |
| C16orf30 | 4210647 | 7.36075 | 0.00115547 |
| C18orf17 | 2070241 | 6.70539 | 0.00201962 |
| C1orf117 | 3170070 | 5.66336 | 0.00498751 |
| C1orf119 | 2680671 | 6.66674 | 0.00208774 |
| C1orf151 | 6380358 | 5.78235 | 0.00449372 |
| C1orf55 | 2680739 | 6.58853 | 0.00223289 |
| C1orf93 | 6250338 | 5.12037 | 0.00805373 |
| C20orf3 | 3610634 | 5.32484 | 0.00671954 |
| C20orf4 | 3140022 | 5.04173 | 0.00863662 |
| C20orf42 | 2350209 | 6.49994 | 0.00240983 |
| C21orf2 | 610653 | 5.08616 | 0.0083022 |
| C21orf33 | 5960301 | 5.72653 | 0.00471878 |
| C2orf25 | 5090204 | 5.67239 | 0.00494816 |
| C2orf28 | 6220487 | 6.63835 | 0.00213927 |
| C3orf37 | 2710544 | 8.24931 | 0.000548537 |
| C6orf108 | 7160164 | 5.11554 | 0.00808831 |
| C6orf149 | 2630181 | 7.52935 | 0.00100209 |
| C6orf150 | 4850370 | 6.2728 | 0.00293222 |
| C6orf66 | 7150601 | 6.20336 | 0.00311408 |
| C6orf72 | 4560474 | 5.17458 | 0.00767554 |
| C8orf1 | 6330471 | 6.32648 | 0.00279912 |
| C8orf33 | 3830278 | 6.16979 | 0.00320608 |
| C9orf10OS | 2100215 | 6.21688 | 0.00307778 |
| C9orf23 | 4200332 | 5.50703 | 0.00572203 |
| C9orf66 | 2710458 | 5.29041 | 0.00692723 |
| CA4 | 3990296 | 5.7511 | 0.00461833 |
| CA5B | 7560162 | 6.64643 | 0.00212447 |
| CACHD1 | 3140142 | 6.30178 | 0.00285957 |
| CALM1 | 1780035 | 5.95878 | 0.00385197 |
| CALU | 1430243 | 5.1151 | 0.00809149 |
| CAMKV | 4610619 | 5.55584 | 0.00548147 |
| CANX | 2230360 | 5.16219 | 0.00776036 |
| CASC2 | 1230753 | 7.35586 | 0.00116026 |
| CASC4 | 6960044 | 5.08649 | 0.00829973 |
| CASP4 | 3610048 | 6.04984 | 0.00355829 |
| CCDC28A | 1050253 | 5.6573 | 0.00501411 |
| CCDC64 | 3420343 | 6.51108 | 0.00238681 |
| CCDC71 | 4850484 | 7.74444 | 0.000836209 |
| CCNA1 | 1660309 | 6.32497 | 0.00280278 |
| CCPG1 | 6960707 | 4.98721 | 0.00906594 |
| CCR2 | 3800270 | 9.70036 | 0.000167225 |
| CCT7 | 7150017 | 6.6909 | 0.00204489 |
| CCT7 | 5050390 | 5.64899 | 0.00505079 |
| CD40LG | 6270128 | 9.49339 | 0.000197683 |
| CD44 | 4060605 | 8.78787 | 0.000351524 |
| CD44 | 1410189 | 6.06527 | 0.00351085 |
| CD55 | 10025 | 5.40211 | 0.00627632 |
| CD58 | 4150161 | 7.26541 | 0.00125265 |
| CD59 | 4040672 | 5.02136 | 0.00879453 |
| CD6 | 4850192 | 7.40781 | 0.00111038 |
| CD9 | 5340246 | 9.38153 | 0.000216455 |
| CD96 | 2100333 | 6.42674 | 0.00256687 |
| CDADC1 | 6660671 | 6.24842 | 0.0029948 |
| CDC25B | 460754 | 7.48683 | 0.00103869 |
| CDCA4 | 2640278 | 5.11739 | 0.00807509 |
| CDK2AP1 | 60575 | 5.49794 | 0.005768 |
| CDK4 | 5270500 | 6.35263 | 0.00273655 |
| CDK5RAP1 | 1440601 | 8.0435 | 0.000651058 |
| CDK5RAP2 | 7000600 | 5.05632 | 0.00852528 |
| CDK9 | 60468 | 5.35508 | 0.00654237 |
| CDS1 | 1240739 | 5.61 | 0.00522667 |
| CEACAM1 | 1780152 | 7.69898 | 0.00086875 |
| CECR1 | 5560280 | 5.86424 | 0.00418323 |
| CENTB2 | 6040152 | 7.16437 | 0.00136482 |
| CEP350 | 6290719 | 7.72495 | 0.000850005 |
| CHIC2 | 3440431 | 7.11256 | 0.00142628 |
| CHMP7 | 5550746 | 7.13009 | 0.00140518 |
| CKAP4 | 6770348 | 6.27439 | 0.00292818 |
| CKAP5 | 2650164 | 5.55448 | 0.00548804 |
| CKLF | 2000551 | 6.07946 | 0.00346781 |
| CLEC4D | 3990328 | 5.49923 | 0.00576145 |
| CLEC4E | 940754 | 6.5595 | 0.00228934 |
| CMTM6 | 2070152 | 8.24662 | 0.000549761 |
| CNTNAP1 | 6350017 | 5.85848 | 0.00420433 |
| CNTNAP2 | 5890273 | 7.11747 | 0.00142034 |
| COG2 | 2810767 | 5.47603 | 0.00588037 |
| COL11A1 | 2750070 | 6.8737 | 0.00174851 |
| COPS7B | 5310050 | 7.49393 | 0.00103249 |
| COPZ1 | 6650403 | 5.66714 | 0.00497101 |
| COQ6 | 7050543 | 7.86382 | 0.000756562 |
| CPA3 | 1400762 | 10.7566 | 7.20E−05 |
| CPA5 | 2030300 | 5.99082 | 0.0037459 |
| CPD | 6590553 | 7.39642 | 0.00112113 |
| CPEB3 | 5290452 | 9.76158 | 0.000159172 |
| CPEB4 | 1690360 | 5.91218 | 0.00401174 |
| CR1 | 610687 | 5.08424 | 0.00831634 |
| CREB5 | 160132 | 8.00844 | 0.000670388 |
| CRELD1 | 10338 | 5.8767 | 0.00413796 |
| CRNKL1 | 1430441 | 6.61176 | 0.00218871 |
| CROCC | 2970440 | 5.32901 | 0.00669484 |
| CRY2 | 1450082 | 5.64809 | 0.00505479 |
| CSNK1A1 | 4850092 | 7.76326 | 0.000823103 |
| CTBS | 110706 | 10.055 | 0.000125747 |
| CUGBP2 | 6110672 | 6.00801 | 0.00369023 |
| CUTA | 2690609 | 7.95362 | 0.000701805 |
| CYB5R4 | 3140039 | 11.3518 | 4.51E−05 |
| CYBASC3 | 1090048 | 6.00055 | 0.00371429 |
| CYP4F3 | 650164 | 9.98926 | 0.000132547 |
| CYSLTR1 | 4810204 | 10.482 | 8.94E−05 |
| DAZAP2 | 1740735 | 5.10312 | 0.00817801 |
| DCXR | 1410369 | 6.73417 | 0.00197038 |
| DDEF1 | 2760349 | 5.18007 | 0.00763827 |
| DDX21 | 6280474 | 5.0642 | 0.00846576 |
| DDX24 | 4280332 | 10.1077 | 0.000120548 |
| DDX39 | 160240 | 5.7799 | 0.00450336 |
| DEGS1 | 6510209 | 7.43248 | 0.00108747 |
| DERPC | 4290673 | 5.20676 | 0.00745972 |
| DFFA | 3520192 | 7.75351 | 0.000829862 |
| DGKA | 6550390 | 4.99306 | 0.00901882 |
| DHPS | 1850541 | 5.524 | 0.00563717 |
| DHPS | 1990390 | 5.00253 | 0.00894309 |
| DHRS9 | 6220450 | 9.8431 | 0.00014906 |
| DICER1 | 6510575 | 6.83755 | 0.00180341 |
| DIP2B | 3990671 | 7.04466 | 0.00151115 |
| DIRC2 | 6020575 | 7.58839 | 0.000953451 |
| DKFZP586D0919 | 4540301 | 7.77622 | 0.000814203 |
| DLX5 | 3360139 | 6.96721 | 0.0016143 |
| DNAJB1 | 2360092 | 6.13066 | 0.00331687 |
| DNAJC3 | 2760064 | 6.87348 | 0.00174883 |
| DNHD1 | 6110142 | 5.77563 | 0.00452023 |
| DPH2 | 2900524 | 6.59316 | 0.00222401 |
| DREV1 | 460142 | 5.28085 | 0.00698604 |
| DSC2 | 7650025 | 6.13809 | 0.00329555 |
| DSTN | 1340689 | 5.10841 | 0.00813973 |
| DTX3L | 2850100 | 7.97473 | 0.000689534 |
| E2F1 | 3940338 | 5.70275 | 0.00481815 |
| ECH1 | 770458 | 5.32501 | 0.00671855 |
| ECHS1 | 3840022 | 7.16026 | 0.00136961 |
| EEF2 | 2750626 | 4.97769 | 0.00914309 |
| EEF2K | 6280343 | 9.13925 | 0.000263638 |
| EFHC2 | 6270129 | 5.45153 | 0.00600869 |
| EIF2AK2 | 1190349 | 5.38063 | 0.00639646 |
| EIF2B1 | 2760563 | 8.06675 | 0.000638551 |
| EIF2C3 | 5860500 | 8.1139 | 0.000613945 |
| EIF3S2 | 7320576 | 7.05567 | 0.00149705 |
| EIF3S4 | 6290431 | 6.62095 | 0.00217151 |
| EIF3S6IP | 4180142 | 7.01892 | 0.00154466 |
| EIF3S7 | 2970468 | 5.19624 | 0.00752953 |
| EIF4A1 | 2970768 | 5.53526 | 0.0055816 |
| EIF4E3 | 1110600 | 12.366 | 2.06E−05 |
| ELF1 | 4250382 | 6.77049 | 0.00190998 |
| EML2 | 2030450 | 5.87838 | 0.00413188 |
| EPB41L2 | 1030189 | 5.59415 | 0.00529995 |
| EPC1 | 2680010 | 8.23987 | 0.000552857 |
| ERO1L | 5270563 | 5.6931 | 0.00485912 |
| EWSR1 | 4780743 | 6.62882 | 0.00215686 |
| EXOC6 | 1940543 | 5.496 | 0.00577784 |
| EXOC7 | 2260520 | 5.56387 | 0.00544292 |
| EXOSC10 | 5490142 | 7.26988 | 0.00124792 |
| EXOSC10 | 5130142 | 5.14398 | 0.0078867 |
| FAIM3 | 2760092 | 5.864 | 0.00418412 |
| FAM102B | 4390468 | 5.23116 | 0.00730015 |
| FAM38A | 2710253 | 4.98385 | 0.00909311 |
| FAM62A | 1110215 | 8.94141 | 0.00030992 |
| FAM91A1 | 4890681 | 5.95153 | 0.00387639 |
| FARS2 | 130403 | 7.40804 | 0.00111017 |
| FBXL13 | 5050653 | 4.97912 | 0.00913145 |
| FBXL15 | 3930687 | 5.67534 | 0.00493537 |
| FBXL20 | 3710484 | 5.53534 | 0.00558121 |
| FBXL5 | 2070377 | 6.25879 | 0.00296803 |
| FBXO11 | 990474 | 5.80831 | 0.0043928 |
| FBXO11 | 6180497 | 5.58782 | 0.00532953 |
| FBXO21 | 3400372 | 6.09855 | 0.00341072 |
| FBXO28 | 3870754 | 7.85148 | 0.000764424 |
| FCRL3 | 4590646 | 5.10631 | 0.00815493 |
| FEZ1 | 360343 | 5.56591 | 0.00543317 |
| FHL1 | 2320475 | 5.4267 | 0.00614167 |
| FLJ10099 | 7100291 | 6.79933 | 0.00186338 |
| FLJ10379 | 5080056 | 5.88741 | 0.00409942 |
| FLJ11795 | 4670056 | 5.4947 | 0.00578447 |
| FLJ20186 | 6940612 | 6.89405 | 0.00171836 |
| FLJ21127 | 3840221 | 5.18932 | 0.00757585 |
| FLJ32028 | 7650379 | 10.0375 | 0.000127518 |
| FLJ32154 | 3940368 | 6.8509 | 0.00178294 |
| FLJ33641 | 5340128 | 5.50724 | 0.00572096 |
| FLJ33790 | 620215 | 5.15728 | 0.00779422 |
| FLJ36268 | 2350372 | 5.37856 | 0.00640813 |
| FLJ38379 | 3870240 | 5.2842 | 0.00696541 |
| FLRT2 | 540358 | 6.58309 | 0.00224336 |
| FN3KRP | 6590386 | 9.18191 | 0.000254624 |
| FNBP1 | 1190470 | 5.4243 | 0.0061547 |
| FNBP1L | 2480255 | 5.58258 | 0.00535414 |
| FNDC3B | 870148 | 6.2997 | 0.00286474 |
| FOXO1A | 270754 | 5.28562 | 0.00695665 |
| FPR1 | 10343 | 5.01605 | 0.00883613 |
| FPRL1 | 3140114 | 5.70404 | 0.00481271 |
| FXYD5 | 6760487 | 5.05011 | 0.00857247 |
| FZD7 | 110343 | 5.83314 | 0.00429847 |
| GABARAPL1 | 2630154 | 8.59363 | 0.000412487 |
| GAGE8 | 6960450 | 5.66586 | 0.0049766 |
| GALNTL5 | 5670747 | 5.81459 | 0.00436875 |
| GALT | 520332 | 11.5689 | 3.81E−05 |
| GATA2 | 3990553 | 5.36069 | 0.00651006 |
| GCA | 940348 | 6.10606 | 0.00338854 |
| GCN5L2 | 130451 | 5.7371 | 0.00467531 |
| GEMIN4 | 4200538 | 6.79701 | 0.00186708 |
| GGA2 | 6270364 | 5.71835 | 0.00475272 |
| GIMAP5 | 6960746 | 8.71348 | 0.000373699 |
| GIMAP6 | 6590523 | 7.40118 | 0.00111662 |
| GIMAP8 | 4540487 | 5.07895 | 0.00835551 |
| GLTSCR2 | 3170092 | 6.54567 | 0.00231676 |
| GNA13 | 2340445 | 5.6436 | 0.00507475 |
| GNAI3 | 5810598 | 5.41555 | 0.00620237 |
| GNAQ | 4760095 | 6.48838 | 0.00243396 |
| GNB1 | 240554 | 5.80351 | 0.00441127 |
| GNB4 | 2470653 | 5.49845 | 0.00576541 |
| GNE | 3610504 | 11.7262 | 3.37E−05 |
| GOLGA3 | 7000041 | 5.16389 | 0.00774865 |
| GOT2 | 1440546 | 7.32571 | 0.00119026 |
| GPBAR1 | 5960035 | 7.31518 | 0.00120092 |
| GPR137B | 7150364 | 5.89303 | 0.00407938 |
| GPR160 | 6180427 | 8.92995 | 0.000312842 |
| GSTM3 | 3940386 | 5.02242 | 0.00878623 |
| GSTM4 | 1030070 | 5.43911 | 0.00607485 |
| GSTP1 | 5420538 | 5.19398 | 0.00754464 |
| GTDC1 | 1990450 | 7.09942 | 0.00144231 |
| H3F3A | 5890307 | 7.44334 | 0.00107754 |
| HAPLN3 | 5360674 | 6.28792 | 0.00289409 |
| HBP1 | 5890494 | 7.1897 | 0.00133577 |
| HDAC1 | 6940242 | 7.58873 | 0.000953175 |
| HELZ | 6290377 | 5.7103 | 0.00478638 |
| HIAT1 | 4060494 | 6.84822 | 0.00178702 |
| HIF1A | 2850288 | 7.36898 | 0.00114746 |
| HIST1H2AC | 4890192 | 7.57549 | 0.00096387 |
| HIST1H2AE | 5860400 | 10.1512 | 0.000116423 |
| HIST1H2BG | 630091 | 5.71961 | 0.0047475 |
| HIST1H2BJ | 5360504 | 5.23354 | 0.00728478 |
| HIST1H3D | 7380241 | 5.50599 | 0.00572724 |
| HIST1H4E | 1780113 | 5.11766 | 0.00807315 |
| HIST1H4H | 4730195 | 11.8236 | 3.12E−05 |
| HIST1H4K | 520097 | 5.95759 | 0.00385597 |
| HIST2H2AA | 1030039 | 5.80666 | 0.00439915 |
| HIST2H2AC | 5860075 | 5.64664 | 0.00506121 |
| HIST2H2BE | 2630451 | 8.31244 | 0.000520526 |
| HLA-DPA1 | 6480500 | 7.56623 | 0.000971415 |
| HLA-DQA1 | 6290561 | 7.38736 | 0.00112975 |
| HLA-DRA | 2680370 | 6.59614 | 0.00221832 |
| HMFN0839 | 7610546 | 6.3451 | 0.00275441 |
| HMG20A | 1710491 | 6.54741 | 0.0023133 |
| HMGB2 | 610563 | 8.05345 | 0.000645674 |
| HNRPF | 2060471 | 5.20123 | 0.00749634 |
| HNRPM | 6270021 | 7.61749 | 0.00093038 |
| HNRPU | 2710026 | 5.97508 | 0.00379761 |
| HPD | 5090554 | 5.83958 | 0.00427435 |
| HSD17B8 | 3130019 | 5.05429 | 0.00854066 |
| HSDL2 | 1340382 | 5.19002 | 0.00757116 |
| HSP90AB1 | 5130082 | 6.59784 | 0.00221508 |
| HSPA1L | 780255 | 6.9121 | 0.00169206 |
| HSPA8 | 1690189 | 7.92263 | 0.000720225 |
| HSPA8 | 6350376 | 5.50264 | 0.00574415 |
| HSPA9B | 4560497 | 5.0627 | 0.00847704 |
| HSPC159 | 4150768 | 6.57979 | 0.00224973 |
| HTATIP2 | 6620674 | 5.3885 | 0.00635215 |
| IBRDC2 | 5910037 | 5.87604 | 0.00414033 |
| ICA1 | 650735 | 5.22617 | 0.00733249 |
| ICAM2 | 1820598 | 8.63677 | 0.000398072 |
| IDH3B | 7380170 | 6.14118 | 0.0032867 |
| IFRD1 | 3780243 | 5.83685 | 0.00428454 |
| IGF2BP3 | 3360433 | 8.47558 | 0.000454733 |
| IGSF8 | 5690576 | 5.08579 | 0.00830487 |
| IL18R1 | 1500328 | 6.13016 | 0.00331834 |
| IL18RAP | 5130475 | 6.18189 | 0.0031726 |
| IL1RAP | 2360398 | 5.59758 | 0.00528402 |
| IL2RB | 1170307 | 6.34396 | 0.00275714 |
| ILF2 | 7400431 | 5.21115 | 0.00743074 |
| ILF3 | 2070494 | 6.00434 | 0.00370205 |
| IMP3 | 1780348 | 8.07729 | 0.000632967 |
| IMP4 | 6380598 | 8.31444 | 0.000519664 |
| IMPDH2 | 3400504 | 6.2529 | 0.0029832 |
| IPO7 | 510746 | 5.09435 | 0.00824198 |
| IRAK3 | 1430762 | 6.40092 | 0.00262472 |
| IRS2 | 6980095 | 5.24759 | 0.00719472 |
| ITCH | 7400369 | 5.17556 | 0.00766886 |
| ITGAM | 6660709 | 5.09625 | 0.0082281 |
| ITGAX | 1240603 | 9.72094 | 0.000164472 |
| ITM2B | 2760358 | 5.41472 | 0.00620692 |
| ITPR2 | 2850377 | 8.07145 | 0.000636057 |
| IVNS1ABP | 2100519 | 5.605 | 0.00524969 |
| JMJD1B | 4120681 | 4.981 | 0.00911615 |
| K-ALPHA-1 | 5900156 | 10.1324 | 0.000118184 |
| KARS | 5900414 | 7.19714 | 0.00132736 |
| KBTBD7 | 2030747 | 6.73079 | 0.0019761 |
| KCMF1 | 4730747 | 4.99997 | 0.00896348 |
| KCNJ15 | 3390458 | 5.79194 | 0.00445614 |
| KCTD17 | 7650605 | 5.68191 | 0.004907 |
| KIAA0174 | 3520168 | 6.5086 | 0.00239193 |
| KIAA0195 | 2190673 | 7.74333 | 0.000836989 |
| KIAA0232 | 1260156 | 5.7984 | 0.00443104 |
| KIAA0692 | 3830390 | 7.83562 | 0.000774646 |
| KIAA0701 | 4060056 | 6.08938 | 0.00343802 |
| KIAA0703 | 1300332 | 6.84384 | 0.00179373 |
| KIAA0859 | 5310754 | 8.57354 | 0.000419383 |
| KIAA0888 | 2490730 | 5.39891 | 0.00629409 |
| KIAA1267 | 2320280 | 5.01711 | 0.00882783 |
| KIAA1344 | 5260674 | 5.96177 | 0.00384194 |
| KIAA1600 | 1770598 | 5.1338 | 0.00795827 |
| KIAA1618 | 7200681 | 9.992 | 0.000132256 |
| KIAA1914 | 4040309 | 6.13932 | 0.00329203 |
| KIAA1961 | 940132 | 8.63697 | 0.000398007 |
| KIF1B | 610465 | 7.07252 | 0.00147572 |
| KLHL8 | 1010754 | 6.18385 | 0.00316722 |
| KPNA4 | 6560377 | 5.20852 | 0.00744804 |
| KREMEN1 | 1440612 | 6.52578 | 0.00235679 |
| KRTAP19-1 | 2710292 | 5.4295 | 0.00612654 |
| KRTAP19-6 | 1450561 | 5.05057 | 0.00856896 |
| L3MBTL2 | 3120301 | 8.99707 | 0.000296114 |
| L3MBTL3 | 5820025 | 5.72882 | 0.00470934 |
| LAMP2 | 3290162 | 10.6146 | 8.05E−05 |
| LARS | 1470762 | 5.0467 | 0.0085985 |
| LAS1L | 1010612 | 5.56266 | 0.00544873 |
| LAX1 | 7000768 | 7.67889 | 0.000883543 |
| LCK | 2230661 | 6.76237 | 0.00192332 |
| LFNG | 3890095 | 5.73123 | 0.0046994 |
| LGR6 | 4760364 | 5.44188 | 0.00606002 |
| LMBRD1 | 4590301 | 5.22412 | 0.00734584 |
| LMNB2 | 5550343 | 5.02955 | 0.00873068 |
| LOC133993 | 4200451 | 5.30894 | 0.00681462 |
| LOC153222 | 1450184 | 8.57901 | 0.000417494 |
| LOC284701 | 20068 | 6.06749 | 0.00350407 |
| LOC285636 | 7610168 | 5.30429 | 0.00684273 |
| LOC343384 | 840347 | 5.51636 | 0.00567521 |
| LOC348645 | 5910682 | 8.05779 | 0.000643342 |
| LOC374395 | 840730 | 6.20241 | 0.00311666 |
| LOC387841 | 3800253 | 5.25844 | 0.00712597 |
| LOC387867 | 1820692 | 5.38172 | 0.00639027 |
| LOC389833 | 6960328 | 6.89012 | 0.00172414 |
| LOC390378 | 6020341 | 5.50622 | 0.0057261 |
| LOC392364 | 770452 | 5.80681 | 0.00439858 |
| LOC400566 | 2570703 | 14.1926 | 5.20E−06 |
| LOC400566 | 3520685 | 7.68717 | 0.000877415 |
| LOC400793 | 3930221 | 5.06119 | 0.00848848 |
| LOC401284 | 3370402 | 8.65103 | 0.000393422 |
| LOC401957 | 2900019 | 5.74489 | 0.00464349 |
| LOC440261 | 3990465 | 6.76775 | 0.00191447 |
| LOC440503 | 940450 | 5.7466 | 0.00463656 |
| LOC441097 | 3800382 | 5.00666 | 0.00891032 |
| LOC51035 | 6480386 | 5.17889 | 0.00764629 |
| LOC51149 | 2750035 | 5.58142 | 0.00535958 |
| LOC57149 | 3830341 | 10.0669 | 0.000124551 |
| LOC642196 | 2030544 | 6.51072 | 0.00238756 |
| LOC642267 | 2970278 | 5.64252 | 0.00507955 |
| LOC642718 | 1940307 | 5.35587 | 0.00653784 |
| LOC642780 | 1580746 | 6.41772 | 0.00258692 |
| LOC642816 | 6110537 | 8.12818 | 0.000606685 |
| LOC642816 | 160458 | 5.29963 | 0.00687096 |
| LOC643060 | 2350121 | 5.3473 | 0.00658753 |
| LOC643300 | 770369 | 5.40606 | 0.00625448 |
| LOC643401 | 2370341 | 7.67203 | 0.000888651 |
| LOC643707 | 3130524 | 5.65439 | 0.00502693 |
| LOC644474 | 4850711 | 8.29745 | 0.000527041 |
| LOC644838 | 2260575 | 6.2576 | 0.00297107 |
| LOC645232 | 4570730 | 7.89079 | 0.000739672 |
| LOC646144 | 2750152 | 5.28085 | 0.00698607 |
| LOC646200 | 6650086 | 5.48605 | 0.00582867 |
| LOC646836 | 5910343 | 5.98222 | 0.00377408 |
| LOC646920 | 1470014 | 5.58907 | 0.00532368 |
| LOC647649 | 2810674 | 5.77173 | 0.00453567 |
| LOC647649 | 2600102 | 4.99758 | 0.00898262 |
| LOC647784 | 7050196 | 5.40878 | 0.00623948 |
| LOC647841 | 1450209 | 7.07418 | 0.00147364 |
| LOC648732 | 7210044 | 5.27582 | 0.00701725 |
| LOC649242 | 3440040 | 5.19455 | 0.00754085 |
| LOC649379 | 540131 | 5.1876 | 0.00758746 |
| LOC649461 | 4150711 | 5.1957 | 0.00753314 |
| LOC650058 | 940706 | 5.29434 | 0.00690319 |
| LOC650557 | 4590563 | 5.59735 | 0.00528509 |
| LOC650849 | 1780543 | 5.84948 | 0.00423752 |
| LOC651076 | 4220138 | 8.28662 | 0.000531802 |
| LOC651131 | 2470092 | 5.05558 | 0.00853092 |
| LOC652025 | 2320309 | 5.13843 | 0.00792564 |
| LOC652219 | 5670121 | 6.92696 | 0.00167072 |
| LOC652455 | 4060138 | 8.99784 | 0.000295927 |
| LOC652458 | 3390725 | 7.11098 | 0.0014282 |
| LOC652578 | 3180192 | 7.76558 | 0.000821499 |
| LOC652759 | 6520333 | 9.81806 | 0.000152094 |
| LOC653063 | 6370463 | 5.34234 | 0.00661643 |
| LOC653181 | 2850274 | 5.43219 | 0.00611199 |
| LOC653492 | 7100092 | 6.10568 | 0.00338965 |
| LOC653518 | 3800082 | 5.52039 | 0.00565514 |
| LOC653610 | 5670544 | 5.39034 | 0.00634187 |
| LOC653832 | 2260446 | 7.31857 | 0.00119748 |
| LOC654123 | 3170491 | 7.01523 | 0.00154953 |
| LOC654123 | 7160477 | 6.54947 | 0.0023092 |
| LOC654123 | 5220112 | 5.91646 | 0.00399679 |
| LOC654126 | 730148 | 6.81786 | 0.00183406 |
| LOC88523 | 3140246 | 6.58819 | 0.00223354 |
| LOC90355 | 10477 | 5.07193 | 0.00840782 |
| LPGAT1 | 870403 | 5.22463 | 0.00734255 |
| LPXN | 4060131 | 7.81314 | 0.000789378 |
| LRDD | 5050307 | 5.63142 | 0.00512928 |
| LRMP | 20553 | 5.26766 | 0.00706808 |
| LRRC42 | 2490397 | 5.32931 | 0.00669307 |
| LRRC4B | 2230019 | 5.35857 | 0.00652225 |
| LRRC8C | 3870102 | 5.62491 | 0.00515871 |
| LRRK2 | 1450523 | 5.57077 | 0.00540998 |
| LRRN1 | 3360156 | 5.28974 | 0.00693133 |
| LRRTM1 | 6980609 | 9.20039 | 0.000250818 |
| LSM4 | 4830563 | 6.26955 | 0.0029405 |
| LUM | 1780215 | 6.16715 | 0.00321345 |
| LXN | 3850669 | 5.5038 | 0.00573829 |
| LY9 | 450037 | 8.33037 | 0.00051284 |
| LY9 | 5310136 | 5.61446 | 0.00520625 |
| LYCAT | 2320241 | 5.8673 | 0.00417206 |
| LYSMD2 | 6550520 | 8.76911 | 0.000356986 |
| LZTR1 | 3140093 | 8.08612 | 0.000628324 |
| MAGED1 | 6480170 | 6.20557 | 0.00310812 |
| MAMDC1 | 670376 | 5.64168 | 0.00508328 |
| MAN1A1 | 4010110 | 5.16872 | 0.00771553 |
| MAN2A2 | 4570612 | 5.60761 | 0.00523765 |
| MAP2K3 | 4150632 | 5.06337 | 0.00847199 |
| MAP2K4 | 6350309 | 7.24078 | 0.00127909 |
| MAP3K4 | 7320594 | 5.47076 | 0.00590772 |
| MAP4K1 | 3420630 | 6.36405 | 0.00270966 |
| MAP7 | 60255 | 7.57404 | 0.000965048 |
| MAPK14 | 6280427 | 5.07097 | 0.00841495 |
| MAX | 1010102 | 5.99342 | 0.00373743 |
| MBP | 2600520 | 6.53858 | 0.00233096 |
| MCM3AP | 5390131 | 8.57304 | 0.000419553 |
| MFNG | 1710286 | 7.46419 | 0.00105873 |
| MGC15619 | 6400563 | 5.92712 | 0.0039598 |
| MGC17624 | 20224 | 6.06151 | 0.00352235 |
| MGC2474 | 4150435 | 5.28563 | 0.0069566 |
| MGC32020 | 7150189 | 5.29166 | 0.00691959 |
| MGC33887 | 1470332 | 5.25207 | 0.00716624 |
| MGC35048 | 7050240 | 5.3186 | 0.00675672 |
| MGC39518 | 5910730 | 5.39515 | 0.006315 |
| MGC57346 | 1340338 | 6.68821 | 0.00204961 |
| MIER1 | 5360575 | 6.61331 | 0.0021858 |
| MIF4GD | 6520241 | 6.53173 | 0.00234475 |
| MIZF | 3400176 | 5.09125 | 0.00826474 |
| MLL2 | 6510349 | 5.16978 | 0.00770831 |
| MLL5 | 4230253 | 9.58735 | 0.000183206 |
| MLLT6 | 2630719 | 6.59288 | 0.00222454 |
| MLR2 | 70022 | 11.0153 | 5.87E−05 |
| MMD | 360671 | 5.48456 | 0.00583633 |
| MME | 240608 | 5.78848 | 0.00446967 |
| MMP9 | 4150224 | 5.11823 | 0.00806906 |
| MNDA | 6380228 | 5.45004 | 0.00601659 |
| MORC2 | 3360364 | 8.66234 | 0.000389771 |
| MRPL34 | 6660253 | 5.09937 | 0.00820532 |
| MRPL44 | 7560014 | 6.03043 | 0.00361891 |
| MRPL49 | 1030692 | 6.7503 | 0.00194331 |
| MRPL9 | 2070131 | 5.46907 | 0.00591651 |
| MRPS26 | 4830435 | 6.08864 | 0.00344022 |
| MS4A2 | 6770427 | 5.60327 | 0.00525768 |
| MSRB3 | 4850414 | 7.72221 | 0.000851962 |
| MTMR11 | 7320195 | 5.19043 | 0.00756841 |
| MTPN | 4880670 | 5.80233 | 0.00441584 |
| MUM1 | 6040259 | 5.02876 | 0.00873684 |
| MUSTN1 | 1260487 | 5.02225 | 0.00878755 |
| MXD1 | 2260239 | 8.31951 | 0.000517481 |
| MYL9 | 4730114 | 5.37621 | 0.00642147 |
| MYLIP | 6370209 | 5.89208 | 0.00408274 |
| MYLK | 6350608 | 10.4778 | 8.97E−05 |
| MYO9A | 7150634 | 9.90968 | 0.000141291 |
| NAP1L4 | 2600286 | 5.30322 | 0.00684918 |
| NBN | 1030398 | 6.47215 | 0.00246826 |
| NCOA1 | 6760121 | 5.29553 | 0.00689592 |
| NDUFA1 | 150132 | 5.18493 | 0.00760541 |
| NDUFA10 | 6480603 | 7.21575 | 0.00130656 |
| NFE2L2 | 6580075 | 7.13461 | 0.00139978 |
| NFIL3 | 6100228 | 5.54302 | 0.00554365 |
| NFKB1 | 4810181 | 7.2131 | 0.00130949 |
| NIPA2 | 270093 | 5.35835 | 0.00652352 |
| NMNAT2 | 1580348 | 7.79232 | 0.000803283 |
| NOLA1 | 1430309 | 5.2716 | 0.00704349 |
| NOLA2 | 1510224 | 5.09396 | 0.00824483 |
| NOSIP | 380685 | 6.3308 | 0.00278869 |
| NOVA1 | 5490133 | 5.25178 | 0.0071681 |
| NPAL3 | 520360 | 5.23525 | 0.00727376 |
| NR2C2 | 5810326 | 6.02484 | 0.00363656 |
| NRBF2 | 5670133 | 5.00611 | 0.00891468 |
| NSUN5 | 5310270 | 5.39767 | 0.00630099 |
| NSUN5C | 2710711 | 5.94789 | 0.00388873 |
| NT5C2 | 520647 | 5.21729 | 0.00739044 |
| NT5C3 | 3780689 | 13.8534 | 6.69E−06 |
| NUBPL | 3840131 | 5.61263 | 0.00521463 |
| NUFIP2 | 5260091 | 6.42347 | 0.00257413 |
| NUMB | 7210692 | 5.65077 | 0.00504289 |
| NUP153 | 1050711 | 5.56317 | 0.00544627 |
| NUP205 | 2750521 | 7.4387 | 0.00108177 |
| NUP210 | 6020500 | 5.6771 | 0.00492774 |
| NUP214 | 730180 | 5.53384 | 0.00558861 |
| NUP43 | 670487 | 5.47064 | 0.00590833 |
| NUP62 | 4760543 | 9.01365 | 0.000292124 |
| NUP85 | 2510132 | 5.30155 | 0.00685933 |
| NUP93 | 50164 | 6.08068 | 0.0034641 |
| OGFOD1 | 2750242 | 5.34015 | 0.00662925 |
| OPLAH | 5820348 | 7.25434 | 0.00126447 |
| OR2D2 | 1500176 | 7.23711 | 0.00128309 |
| OSBP | 6760441 | 9.34991 | 0.000222086 |
| OSTM1 | 6860376 | 5.32043 | 0.00674579 |
| OTUD1 | 5490064 | 8.75379 | 0.000361512 |
| P15RS | 4390768 | 5.23825 | 0.00725449 |
| P2RX4 | 2060332 | 5.08025 | 0.00834588 |
| P2RY11 | 7330487 | 5.78673 | 0.00447653 |
| P2RY2 | 5900446 | 5.10762 | 0.00814544 |
| PABPC4 | 6550142 | 6.71407 | 0.00200464 |
| PADI2 | 6110133 | 5.85279 | 0.00422528 |
| PADI4 | 5310653 | 7.07126 | 0.00147731 |
| PAK2 | 2060279 | 10.9714 | 6.07E−05 |
| PAK2 | 4060722 | 6.38767 | 0.00265494 |
| PBEF1 | 3800243 | 6.95946 | 0.00162501 |
| PCDHGB7 | 1190139 | 5.09031 | 0.00827161 |
| PCNT2 | 2480082 | 5.33655 | 0.00665035 |
| PCSK2 | 7150273 | 5.93808 | 0.00392212 |
| PCSK7 | 1400270 | 6.42419 | 0.00257251 |
| PDCD11 | 7160296 | 8.17861 | 0.000581747 |
| PDE5A | 6940524 | 10.1285 | 0.000118562 |
| PDLIM5 | 520730 | 7.30039 | 0.00121606 |
| PDLIM7 | 2680682 | 5.1749 | 0.00767336 |
| PDZD8 | 5720398 | 7.77266 | 0.000816636 |
| PELI1 | 1780672 | 6.67833 | 0.00206706 |
| PELI2 | 270717 | 9.87636 | 0.000145125 |
| PEX19 | 1450414 | 6.11524 | 0.0033616 |
| PFTK1 | 2000390 | 8.68448 | 0.000382729 |
| PGK1 | 6980129 | 7.83973 | 0.000771983 |
| PGM2 | 2710528 | 5.6691 | 0.00496244 |
| PHF10 | 4260053 | 5.51189 | 0.00569759 |
| PHF15 | 3420735 | 6.81634 | 0.00183644 |
| PHF19 | 4540082 | 8.35459 | 0.000502643 |
| PHF20L1 | 430246 | 5.08044 | 0.00834448 |
| PHTF1 | 1070189 | 4.97601 | 0.00915682 |
| PIGR | 6940333 | 5.51367 | 0.00568866 |
| PIP5K2B | 5090477 | 12.1358 | 2.45E−05 |
| PITPNB | 2100615 | 5.9563 | 0.00386031 |
| PLOD2 | 3710228 | 5.08258 | 0.00832865 |
| PLP2 | 2320717 | 6.91779 | 0.00168386 |
| PLSCR1 | 1260228 | 6.81029 | 0.00184597 |
| PMS2CL | 5560484 | 5.15595 | 0.0078034 |
| PMVK | 3460242 | 5.34356 | 0.00660932 |
| PNPO | 3780220 | 5.54949 | 0.00551216 |
| POLE3 | 4260154 | 5.23073 | 0.00730295 |
| POMT1 | 4880681 | 5.2558 | 0.00714264 |
| PPBP | 6350364 | 7.50401 | 0.00102374 |
| PPP1R16B | 6040196 | 5.18776 | 0.00758634 |
| PPP2R5A | 650767 | 8.71872 | 0.000372093 |
| PPP4R1 | 610408 | 5.77944 | 0.00450515 |
| PPP6C | 4040278 | 5.15807 | 0.00778875 |
| PPRC1 | 20647 | 7.34854 | 0.00116747 |
| PRG1 | 650541 | 6.42167 | 0.00257811 |
| PRKAR1A | 4260035 | 5.05363 | 0.00854571 |
| PRKCB1 | 4070215 | 7.50526 | 0.00102266 |
| PRO0149 | 4230463 | 6.1469 | 0.00327043 |
| PROSC | 3990176 | 8.25426 | 0.000546284 |
| PRPF8 | 4590082 | 5.72624 | 0.00472001 |
| PRPS2 | 360685 | 5.56784 | 0.00542396 |
| PRR3 | 7000408 | 7.18471 | 0.00134144 |
| PRRG4 | 6980100 | 5.24229 | 0.00722857 |
| PRSS12 | 1580168 | 9.32773 | 0.000226126 |
| PRSS15 | 1820341 | 5.02946 | 0.00873134 |
| PRUNE | 4560039 | 11.4293 | 4.24E−05 |
| PSD3 | 650059 | 5.60351 | 0.00525656 |
| PSMD2 | 5720497 | 6.50393 | 0.00240156 |
| PSRC2 | 5700164 | 6.32396 | 0.00280522 |
| PTEN | 1500717 | 5.95378 | 0.00386878 |
| PTPLAD1 | 1110110 | 6.42377 | 0.00257344 |
| PTPN1 | 2760603 | 6.12264 | 0.00334007 |
| PTPRC | 2570379 | 5.50824 | 0.00571592 |
| PUM2 | 2490037 | 7.03768 | 0.00152017 |
| PURA | 2360367 | 6.13054 | 0.00331722 |
| QKI | 6660097 | 10.4177 | 9.41E−05 |
| QPCT | 4780672 | 6.79636 | 0.00186812 |
| RAB22A | 6400372 | 6.32792 | 0.00279564 |
| RAB31 | 7570603 | 9.73654 | 0.000162417 |
| RAB3GAP2 | 6400292 | 7.04517 | 0.00151049 |
| RAB9B | 1580626 | 7.42679 | 0.00109271 |
| RAD50 | 7100059 | 5.58997 | 0.00531948 |
| RAG2 | 150100 | 5.03447 | 0.00869253 |
| RAMP2 | 6620612 | 6.77163 | 0.00190812 |
| RANGAP1 | 3710189 | 5.68851 | 0.0048787 |
| RAP1A | 3060692 | 5.33052 | 0.0066859 |
| RARRES3 | 5720458 | 4.98807 | 0.00905894 |
| RARS | 7150739 | 5.13801 | 0.00792862 |
| RASSF3 | 7160494 | 6.25654 | 0.00297381 |
| RBBP5 | 1740133 | 5.94426 | 0.00390105 |
| RBM14 | 2970332 | 6.40973 | 0.00260482 |
| RBM21 | 360402 | 5.68763 | 0.00488246 |
| RBM4 | 620722 | 7.14981 | 0.00138182 |
| RBM4 | 3990072 | 5.3392 | 0.0066348 |
| RBMX | 5690673 | 6.09306 | 0.00342704 |
| RCC2 | 510450 | 11.1079 | 5.46E−05 |
| REPS1 | 3420725 | 5.11173 | 0.00811578 |
| REPS2 | 6590349 | 5.40985 | 0.0062336 |
| RFC5 | 730592 | 5.05912 | 0.00850412 |
| RFFL | 5870551 | 6.14532 | 0.00327494 |
| RFWD2 | 3870543 | 5.3497 | 0.00657357 |
| RFX4 | 3120181 | 5.07575 | 0.00837933 |
| RFX5 | 2640373 | 5.78564 | 0.00448078 |
| RINT-1 | 50709 | 6.25339 | 0.00298192 |
| RIPK2 | 5690093 | 6.77601 | 0.00190097 |
| RNASEL | 4180079 | 5.13886 | 0.00792264 |
| RNF122 | 5900333 | 5.2518 | 0.00716798 |
| RNF13 | 4280047 | 7.83493 | 0.000775091 |
| RNF149 | 10082 | 6.99312 | 0.00157901 |
| RNF38 | 6220022 | 8.75023 | 0.000362573 |
| ROCK2 | 50521 | 7.68464 | 0.000879281 |
| RPAP1 | 6860243 | 6.53879 | 0.00233053 |
| RPL8 | 6380148 | 5.57074 | 0.00541014 |
| RPS5 | 4280326 | 5.00491 | 0.00892418 |
| RPS6KA5 | 2030482 | 9.75937 | 0.000159455 |
| RPUSD3 | 1990673 | 6.11708 | 0.00335624 |
| RRM2B | 5390100 | 6.63373 | 0.00214778 |
| RSBN1L | 6420692 | 8.16086 | 0.000590399 |
| RTN3 | 4280463 | 5.50049 | 0.00575505 |
| RUNX1 | 2100427 | 7.98415 | 0.000684129 |
| RUTBC3 | 2690576 | 7.20305 | 0.00132072 |
| RUVBL1 | 3520082 | 9.37607 | 0.000217416 |
| RUVBL1 | 2750408 | 6.86356 | 0.00176373 |
| S100A8 | 6280576 | 6.21243 | 0.00308969 |
| S100P | 2640609 | 5.18716 | 0.00759042 |
| SAE1 | 5690008 | 5.52614 | 0.00562656 |
| SAMHD1 | 7320047 | 8.26185 | 0.000542851 |
| SAMM50 | 990273 | 6.25095 | 0.00298823 |
| SAMSN1 | 150632 | 7.20813 | 0.00131504 |
| SAP30 | 2510133 | 6.81248 | 0.00184252 |
| SCAMPS | 3370687 | 6.69057 | 0.00204547 |
| SDCCAG3 | 1340731 | 6.01652 | 0.00366301 |
| SDHD | 6650754 | 5.08243 | 0.00832975 |
| SEC31L1 | 6040037 | 7.76422 | 0.00082244 |
| SEC31L2 | 4010673 | 5.57648 | 0.0053829 |
| SEH1L | 430142 | 7.53442 | 0.000997814 |
| SEL1L | 4280661 | 5.5022 | 0.00574641 |
| SERPINC1 | 7400240 | 6.84944 | 0.00178516 |
| SF3B3 | 160682 | 5.74597 | 0.00463913 |
| SFRS15 | 7320273 | 5.89798 | 0.00406178 |
| SHMT2 | 2710278 | 6.02934 | 0.00362234 |
| SIAHBP1 | 4900053 | 9.06693 | 0.000279673 |
| SIGIRR | 7380328 | 6.31133 | 0.00283606 |
| SIPA1L2 | 3370605 | 5.96247 | 0.00383959 |
| SIRPB2 | 6280754 | 7.50861 | 0.00101977 |
| SLC10A5 | 840332 | 5.5181 | 0.00566652 |
| SLC11A1 | 1430292 | 8.23133 | 0.000556792 |
| SLC17A5 | 1570543 | 6.16491 | 0.00321972 |
| SLC22A4 | 2710397 | 6.02835 | 0.00362547 |
| SLC24A5 | 1660392 | 5.38386 | 0.00637822 |
| SLC25A25 | 130113 | 8.24117 | 0.00055226 |
| SLC25A3 | 4050398 | 6.23269 | 0.00303589 |
| SLC25A5 | 7550537 | 10.638 | 7.90E−05 |
| SLC27A2 | 6110328 | 6.18193 | 0.0031725 |
| SLC2A11 | 2750091 | 6.6354 | 0.00214469 |
| SLC36A1 | 7100136 | 8.76079 | 0.000359438 |
| SLC36A4 | 2350195 | 5.00374 | 0.00893352 |
| SLC37A3 | 2230008 | 4.99148 | 0.00903155 |
| SLC39A1 | 2630400 | 5.44178 | 0.00606057 |
| SLC40A1 | 840427 | 6.31551 | 0.00282581 |
| SLC7A6 | 2480402 | 5.78558 | 0.00448103 |
| SLC9A3R1 | 6060324 | 6.16182 | 0.00322836 |
| SMAD3 | 5130767 | 6.04884 | 0.00356138 |
| SMAP1 | 4040747 | 5.04835 | 0.00858592 |
| SMARCA3 | 7380576 | 5.29651 | 0.00688997 |
| SMC1L1 | 1500040 | 5.89165 | 0.00408427 |
| SMCHD1 | 5700136 | 6.32478 | 0.00280324 |
| SMOC1 | 7100685 | 6.5461 | 0.00231591 |
| SNRP70 | 2070468 | 5.18375 | 0.00761339 |
| SOD1 | 2120324 | 5.66781 | 0.00496808 |
| SPAG9 | 4290477 | 8.74933 | 0.000362839 |
| SPAG9 | 380541 | 6.49845 | 0.00241294 |
| SPAST | 4830082 | 6.1523 | 0.00325515 |
| SPATA20 | 4120133 | 7.02394 | 0.00153806 |
| SPTBN1 | 4480091 | 6.5738 | 0.00226136 |
| SRP68 | 6020402 | 7.87443 | 0.000749867 |
| SRPK1 | 3460674 | 11.5415 | 3.89E−05 |
| SRRM1 | 290707 | 7.63769 | 0.000914697 |
| SSR2 | 2650240 | 5.6578 | 0.00501189 |
| SSX2 | 4120088 | 5.46733 | 0.00592563 |
| STK17B | 2100035 | 9.32157 | 0.00022726 |
| STK25 | 1820142 | 5.31426 | 0.00678266 |
| STK4 | 2680209 | 7.26555 | 0.00125251 |
| STX3A | 3290192 | 5.22428 | 0.0073448 |
| STXBP5 | 3460189 | 9.56076 | 0.000187189 |
| SVIL | 4280373 | 8.94209 | 0.000309748 |
| SYT17 | 730725 | 5.49774 | 0.00576898 |
| TACC1 | 1050605 | 6.76996 | 0.00191084 |
| TAF15 | 5960128 | 9.53807 | 0.000190658 |
| TAF1C | 3850025 | 5.70477 | 0.00480966 |
| TARSL1 | 6480328 | 6.05332 | 0.00354754 |
| TDRD7 | 7200682 | 6.13568 | 0.00330244 |
| TFEC | 990377 | 5.20654 | 0.00746112 |
| TFF3 | 5550224 | 4.99123 | 0.00903355 |
| TGIF2 | 4850438 | 5.97846 | 0.00378645 |
| THBD | 5490348 | 6.35169 | 0.00273877 |
| TIPARP | 5720681 | 8.71399 | 0.000373542 |
| TLE4 | 6290170 | 7.77483 | 0.000815154 |
| TLN2 | 6520086 | 6.18316 | 0.00316913 |
| TLR4 | 4390615 | 6.55034 | 0.00230748 |
| TLR8 | 6550307 | 6.25081 | 0.0029886 |
| TLR8 | 510338 | 5.21895 | 0.00737959 |
| TM6SF1 | 10541 | 9.23945 | 0.000242963 |
| TM9SF2 | 2810110 | 5.11562 | 0.00808776 |
| TMCC3 | 2650152 | 6.73706 | 0.00196549 |
| TMCO3 | 3130091 | 5.70812 | 0.00479554 |
| TMED2 | 7560445 | 6.6213 | 0.00217084 |
| TMED7 | 4230504 | 6.34151 | 0.00276298 |
| TMEM109 | 4880364 | 6.8448 | 0.00179226 |
| TMEM127 | 670079 | 5.17027 | 0.00770495 |
| TMEM49 | 4010358 | 5.21371 | 0.00741391 |
| TMEM87B | 7320669 | 6.27742 | 0.00292052 |
| TMEM99 | 1090041 | 5.36207 | 0.00650214 |
| TNFAIP6 | 2370524 | 19.2839 | 1.41E−07 |
| TNFRSF10A | 4150739 | 5.42505 | 0.00615059 |
| TNFRSF10B | 6450767 | 6.74205 | 0.0019571 |
| TNFSF13B | 460608 | 7.80431 | 0.000795241 |
| TNFSF4 | 1440341 | 7.43781 | 0.00108258 |
| TNRC6B | 2750386 | 6.77666 | 0.00189992 |
| TOR1AIP1 | 3180041 | 8.12925 | 0.000606148 |
| TRA16 | 6650541 | 5.54381 | 0.00553978 |
| TRAF3IP3 | 4640528 | 6.8101 | 0.00184628 |
| TRAP1 | 160736 | 7.4632 | 0.00105962 |
| TRAPPC6A | 1980424 | 5.75576 | 0.00459954 |
| TRFP | 460524 | 5.09703 | 0.00822237 |
| TRIADS | 2190524 | 5.81043 | 0.00438465 |
| TRIB1 | 2710044 | 5.41006 | 0.00623249 |
| TRIM25 | 2850576 | 5.54455 | 0.00553618 |
| TSP50 | 5690037 | 10.1995 | 0.000112016 |
| TSPAN2 | 1770131 | 10.7267 | 7.37E−05 |
| TTC4 | 7100504 | 5.24019 | 0.00724201 |
| TXNDC13 | 1940259 | 6.28066 | 0.00291234 |
| TXNDC14 | 380315 | 5.8647 | 0.00418154 |
| TXNRD1 | 7050372 | 5.15744 | 0.00779313 |
| TYRP1 | 3360491 | 5.46528 | 0.00593633 |
| UBC | 4780609 | 5.79074 | 0.00446082 |
| UBE2C | 2450603 | 6.82453 | 0.00182361 |
| UBE2G1 | 430382 | 12.9455 | 1.32E−05 |
| UBE2G2 | 1440382 | 6.41917 | 0.00258368 |
| UBE2I | 460273 | 6.89336 | 0.00171937 |
| UBE2J1 | 3840446 | 5.22299 | 0.00735319 |
| UBE2Z | 2510639 | 9.21474 | 0.000247902 |
| UBE2Z | 7160767 | 8.12999 | 0.000605771 |
| UBL3 | 130609 | 6.5499 | 0.00230835 |
| UBUCP1 | 1500202 | 6.45134 | 0.00251296 |
| UBQLN3 | 7100392 | 6.00542 | 0.00369856 |
| UBQLN4 | 990224 | 5.79508 | 0.00444393 |
| UGCGL1 | 7210372 | 13.0538 | 1.22E−05 |
| UIP1 | 1070377 | 5.01976 | 0.00880705 |
| UMPS | 3990196 | 6.2988 | 0.00286697 |
| UNC84B | 5570750 | 5.13179 | 0.00797253 |
| USP10 | 1980021 | 5.78018 | 0.00450224 |
| USP3 | 7570112 | 5.50183 | 0.00574825 |
| USP37 | 6840646 | 5.38618 | 0.00636519 |
| USP52 | 1740576 | 6.06563 | 0.00350976 |
| USP8 | 270750 | 6.47431 | 0.00246367 |
| USP9X | 2680064 | 7.11503 | 0.00142329 |
| UTX | 270731 | 6.79812 | 0.00186531 |
| VCL | 6840039 | 5.52267 | 0.00564376 |
| VDAC2 | 1770379 | 7.37165 | 0.00114487 |
| VPREB3 | 360066 | 5.35198 | 0.00656035 |
| VPS11 | 6330634 | 5.1734 | 0.00768355 |
| VPS13A | 6900392 | 5.69895 | 0.00483424 |
| WDFY3 | 5360349 | 5.3433 | 0.00661081 |
| WDR54 | 5700403 | 7.0291 | 0.00153132 |
| WDR6 | 5900021 | 5.61681 | 0.00519549 |
| WHSC1 | 7100520 | 9.59956 | 0.000181408 |
| WNT3A | 270402 | 7.20424 | 0.00131938 |
| WRB | 6420138 | 5.939 | 0.00391897 |
| WSB1 | 5260673 | 5.23028 | 0.00730589 |
| WWP2 | 1190100 | 6.28449 | 0.00290269 |
| XPO4 | 870370 | 7.93163 | 0.000714824 |
| XPO5 | 3130711 | 6.49421 | 0.00242177 |
| XPR1 | 5910093 | 5.2559 | 0.007142 |
| XTP3TPA | 1430156 | 6.17176 | 0.00320062 |
| YARS2 | 1010341 | 5.217 | 0.00739229 |
| YIPF4 | 5290289 | 8.3261 | 0.000514659 |
| YTHDF3 | 1400484 | 5.00651 | 0.00891149 |
| YWHAZ | 7210056 | 6.06452 | 0.00351313 |
| ZBTB24 | 6660689 | 6.16942 | 0.00320714 |
| ZBTB34 | 4070286 | 5.63575 | 0.00510983 |
| ZBTB40 | 6380687 | 5.44163 | 0.00606134 |
| ZBTB9 | 3290019 | 6.02095 | 0.00364891 |
| ZFP91 | 2450064 | 9.33947 | 0.000223977 |
| ZFYVE20 | 4050273 | 5.15079 | 0.0078392 |
| ZMPSTE24 | 5490408 | 5.82224 | 0.00433961 |
| ZMYM6 | 7380274 | 8.67458 | 0.000385862 |
| ZNF161 | 6960390 | 6.82473 | 0.00182331 |
| ZNF200 | 6290458 | 9.18631 | 0.000253711 |
| ZNF207 | 4230373 | 5.54984 | 0.00551046 |
| ZNF268 | 6020132 | 5.41912 | 0.00618285 |
| ZNF313 | 4490747 | 6.13144 | 0.00331463 |
| ZNF416 | 6250047 | 5.63131 | 0.0051298 |
| ZNF589 | 3170468 | 6.04231 | 0.00358169 |
| ZNF599 | 150360 | 5.5351 | 0.00558241 |
| ZNF654 | 6200563 | 9.10959 | 0.000270097 |
| ZNF654 | 2060370 | 5.56102 | 0.00545658 |
| ZNF740 | 4920575 | 7.9771 | 0.000688171 |

TABLE 6

| Probe set | Gene name |
|---|---|
| 3780689 | NT5C3 |
| 3830341 | LYRM1 |
| 2370524 | TNFAIP6 |
| 7200681 | XM_941239.1 |
| AUC = 0.8, n = 4 | |
| 3780689 | NT5C3 |
| 1110600 | EIF4E3 |
| 430382 | UBE2G1 |
| 2370524 | TNFAIP6 |
| 3830341 | LYRM1 |
| 1770131 | TSPAN2 |
| 3360433 | IGF2BP3 |
| 2570703 | LOC400566 |
| 3460674 | SRPK1 |
| AUC = 0.82, n = 9 | |
| 3780689 | NT5C3 |
| 1110600 | EIF4E3 |
| 430382 | UBE2G1 |
| 2370524 | TNFAIP6 |
| 3830341 | LYRM1 |
| 1770131 | TSPAN2 |
| 3360433 | IGF2BP3 |
| 2570703 | LOC400566 |
| 3460674 | SRPK1 |
| 6180427 | GPR160 |
| AUC = 0.85, n = 10 | |
| 3780689 | NT5C3 |
| 3830341 | LYRM1 |
| 2370524 | TNFAIP6 |
| 7200681 | XM_941239.1 |
| 6180427 | GPR160 |
| 6200563 | ZNF654 |
| 3140039 | CYB5R4 |
| 430382 | UBE2G1 |
| 5490064 | OTUD |
| 4290477 | SPAG9 |
| 6550520 | LYSMD2 |
| 3460189 | STXBP5 |
| 4280332 | DDX24 |
| AUC = 0.9, n = 13 | |
| 3780689 | NT5C3 |
| 3830341 | LYRM1 |
| 2370524 | TNFAIP6 |
| 7200681 | NA |
| 6180427 | GPR160 |
| 6200563 | ZNF654 |
| 3140039 | CYB5R4 |
| 430382 | UBE2G1 |
| 5490064 | NA |
| 4290477 | NA |
| 6550520 | LYSMD2 |
| 3460189 | STXBP5 |
| 4280332 | DDX24 |
| 2570703 | LOC400566 |
| 3610504 | GNE |
| 270717 | PELI2 |
| 3180041 | TOR1AIP1 |
| 520332 | GALT |
| 2850100 | DTX3L |
| 4730195 | HIST1H4H |
| 3360433 | IGF2BP3 |
| 6420692 | RSBN1L |
| 6220450 | DHRS9 |
| 4060138 | NA |
| 7040187 | ARRDC4 |
| 5860500 | EIF2C3 |
| 460608 | TNFSF13B |
| 5860400 | HIST1H2AE |
| 3460674 | SRPK1 |
| AUC = 0.9, n = 29 | |

TABLE 7

| Probe set | Gene name |
|---|---|
| 8960440 | DEFA4 |
| 10279 | S100A12 |
| 990097 | CEACAM8 |
| 2370524 | TNFAIP6 |
| AUC = 0.81, n = 4 | |
| 6960440 | DEFA4 |
| 10279 | S100A12 |
| 990097 | CEACAM8 |
| 1090427 | LOC653600 |
| 1580259 | LOC389787 |
| 6960554 | LCN2 |
| 4390242 | DEFA1 |
| 3780689 | NT5C3 |
| 2370524 | TNFAIP6 |
| AUC = 0.84, n = 9 | |
| 6960440 | DEFA4 |
| 10279 | S100A12 |
| 990097 | CEACAM8 |
| 1090427 | LOC653600 |
| 1580259 | LOC389787 |
| 6960554 | LCN2 |
| 4390242 | DEFA1 |
| 3460674 | SRPK1 |
| 3780689 | NT5C3 |
| 2370524 | TNFAIP6 |
| AUC = 0.86, n = 10 | |
| 6960440 | DEFA4 |
| 10279 | S100A12 |
| 990097 | CEACAM8 |
| 1090427 | LOC653600 |
| 1580259 | LOC389787 |
| 6960554 | LCN2 |
| 4390242 | DEFA1 |
| 6330376 | CA1 |
| 6350364 | PPBP |
| 4250035 | RAP1GAP |
| 3460674 | SRPK1 |
| 3780689 | NT5C3 |
| 2370524 | TNFAIP6 |
| AUC = 0.91, n = 13 | |
| 6960440 | DEFA4 |
| 10279 | S100A12 |
| 990097 | CEACAM8 |
| 1090427 | LOC653600 |
| 1580259 | LOC389787 |
| 6960554 | LCN2 |
| 4390242 | DEFA1 |
| 6330376 | CA1 |
| 6350364 | PPBP |
| 4250035 | RAP1GAP |
| 4060066 | ITGA2B |
| 5900072 | LOC347376 |
| 6400736 | CAMP |
| 1470554 | ELA2 |
| 6980537 | HS.291319 |
| 6860754 | ARG1 |
| 2810040 | APOBEC3A |
| 1190349 | EIF2AK2 |
| 5080398 | TLR1 |
| 3140039 | CYB5R4 |
| 3180041 | TOR1AIP1 |
| 4730195 | HIST1H4H |
| 460608 | TNFSF13B |
| 3460189 | STXBP5 |
| 3610504 | GNE |
| 4280332 | DDX24 |
| 3460674 | SRPK1 |
| 3780689 | NT5C3 |
| 2370524 | TNFAIP6 |
| AUC = 0.91, n = 29 | |

TABLE 8

| Probe set | gene name |
|---|---|
| 6960440 | DEFA4 |
| 10279 | S100A12 |
| 990097 | CEACAM8 |
| 1090427 | LOC653600 |
| AUC = 0.82, n = 4 | |
| 6960440 | DEFA4 |
| 10279 | S100A12 |
| 990097 | CEACAM8 |
| 1090427 | LOC653600 |
| 1580259 | LOC389787 |
| 6960554 | LCN2 |
| 4390242 | DEFA1 |
| 6330376 | CA1 |
| 6350364 | PPBP |
| AUC = 0.85, n = 9 | |
| 6960440 | DEFA4 |
| 10279 | S100A12 |
| 990097 | CEACAM8 |
| 1090427 | LOC653600 |
| 1580259 | LOC389787 |
| 6960554 | LCN2 |
| 4390242 | DEFA1 |
| 6330376 | CA1 |
| 6350364 | PPBP |
| 4250035 | RAP1GAP |
| AUC = 0.89, n = 10 | |
| 6960440 | DEFA4 |
| 10279 | S100A12 |
| 990097 | CEACAM8 |
| 1090427 | LOC653600 |
| 1580259 | LOC389787 |
| 6960554 | LCN2 |
| 4390242 | DEFA1 |
| 6330376 | CA1 |
| 6350364 | PPBP |
| 4250035 | RAP1GAP |
| 4060066 | ITGA2B |
| 5900072 | LOC347376 |
| 6400736 | CAMP |
| AUC = 0.93, n = 13 | |
| 6960440 | DEFA4 |
| 10279 | S100A12 |
| 990097 | CEACAM8 |
| 1090427 | LOC653600 |
| 1580259 | LOC389787 |
| 6960554 | LCN2 |
| 4390242 | DEFA1 |
| 6330376 | CA1 |
| 6350364 | PPBP |
| 4250035 | RAP1GAP |
| 4060066 | ITGA2B |
| 5900072 | LOC347376 |
| 6400736 | CAMP |
| 1470554 | ELA2 |
| 6980537 | HS.291319 |
| 6860754 | ARG1 |
| 2810040 | APOBEC3A |
| 1190349 | EIF2AK2 |
| 5080398 | TLR1 |
| 2680273 | ZFP36L1 |
| 520646 | BLVRB |
| 2340110 | MGC13057 |
| 4120707 | RPL23 |
| 7650678 | FAM46C |
| 430328 | ERAF |
| 5050075 | FTHL12 |
| 2650440 | FTHL2 |
| 6450692 | FAM104A |
| 4880717 | ACSL1 |
| AUC = 0.94, n = 29 | |

### Examples

### Material and Methods

### Cases and Controls

Lung cancer cases and controls were recruited at the University Hospital Cologne and the Lung Clinic Merheim, Cologne, Germany. Prevalent lung cancer cases and controls were recruited in two hospitals in Cologne, Germany (University Hospital Cologne, Lung Clinic Merheim) within two genetic-epidemiological case control trials (Lung Cancer Study (LuCS) and Cologne Smoking Study (CoSmoS)). A case was defined by the pathological diagnosis of non-small-cell lung cancer or small-cell lung cancer by histology or cytology. A control was defined by the absence of lung cancer at any time-point of the patient's history. Individuals were not accepted as controls if they actually suffered from a cancer of the upper respiratory tract, the upper gastrointestinal tract or the urogenital system, since smoking represents a risk factor for the development of these cancer entities. An individual was not accepted for the control group if the reason for admission was an acute exacerbation of a chronic obstructive pulmonary disease or an acute cardiovascular event (heart attack, cerebral ischemia). These exclusion criteria were due to the simultaneous analysis of risk factors for acute cardiovascular events in this epidemiological study.

Lung cancer cases were primarily recruited in the Department of Haematology and Oncology (Department I for Internal Medicine, University Hospital Cologne) and in the Department of Thoracic Surgery (Lung Clinic Merheim). In order to recruit individuals with comparable comorbidity, the inventors used in-patient controls that were primarily recruited in the Department of Dermatology and Venerology and in the Department of Orthopaedics and Trauma Surgery at the University Hospital Cologne. Comorbidity of cases and controls was assessed using the medical records of the patients without performing additional examinations. Overall, the median age in this study was 65.74 years for the lung cancer patients and 63.92 years for the controls, respectively.

Initially, PAXgene stabilized blood samples from two independent groups of prevalent lung cancer cases and controls (prevalent groups; PG1: n=84, PG2: n=24) were used to establish and validate a lung cancer specific classifier. Blood was taken prior chemotherapy in all patients. Matching was performed for age (+/−5 years), gender and pack years (+/−5) (Tables 1 and 2). An additional prevalent group of cases and controls (PG3, n=43) was built without matching and used for further validation of the classifier. Analyses were approved by the local ethics committee and all probands gave informed consent (Tables 1 and 2). Overall, in the group of controls, the inventors recruited 12 individuals suffering from advanced chronic obstructive lung disease as typically seen in a population of heavily smoking adults. Other diseases such as hypertension (n=28) or cardiac diseases (n=6) were observed in the control group. The inventors further included patients with other malignancies (n=13) (skin=10, prostate=2, brain=1). The mean age was 60 for the individuals without lung cancer and 62 for those with lung cancer, respectively (T test: p=0.12).

Blood Collection and cRNA Synthesis and Array Hybridization

2.5 ml blood were drawn into PAXgene vials. After RNA isolation biotin labeled cRNA preparation was performed using the Ambion® Illumina RNA amplification kit (Ambion, UK) and Biotin-16-UTP (10 mmol/1; Roche Molecular Biochemicals) or Illumina® TotalPrep RNA Amplification Kit (Ambion, UK). 1.5 μg of biotin labeled cRNA was hybridized to Sentrix® whole genome bead chips WG6 version 2, (Illumina, USA) and scanned on the Illumina® BeadStation 500×. For data collection, the inventors used Illumina® BeadStudio 3.1.1.0 software. Data are available at http://www.ncbi.nlm.nih.gov/geo/GSE12771).

### Quality Control

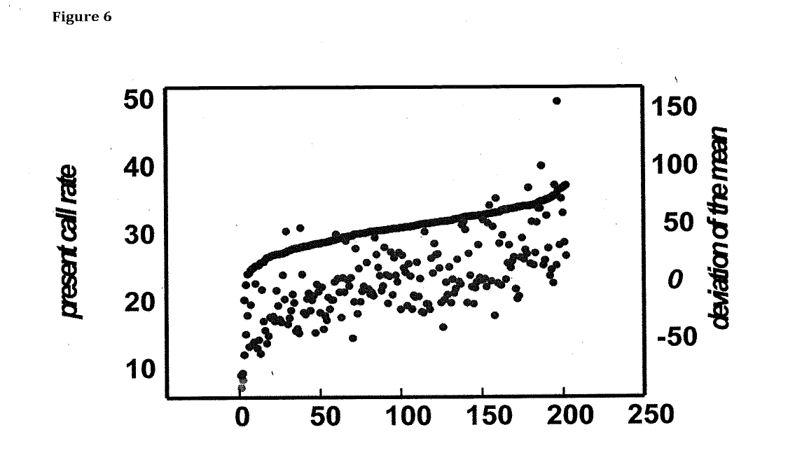

For RNA quality control, the ratio of the OD at wavelengths of 260 nm and 280 nm was calculated and only samples with an OD between 1.85 and 2.1 were further processed. To determine the quality of cRNA, a semi-quantitative RT-PCR amplifying a 5′prime and a 3′prime product of the β-actin gene was used as previously described (Zander T, Yunes J A, Cardoso A A, Nadler L M. Rapid, reliable and inexpensive quality assessment of biotinylated cRNA. Braz J Med Biol Res 2006; 39: 589-93). Quality of RNA expression data was controlled by different separate tools. First, the inventors performed quality control by visual inspection of the distribution of raw expression values. Therefore, the inventors constructed pairwise scatterplots of expression values from all arrays (R-project Vs 2.8.0) (Team RDC. R: A language and environment for statistical computing. R Foundation for Statistical Computing, 2006.). For data derived from an array of good quality a high correlation of expression values is expected leading to a cloud of dots along the diagonal. Secondly, the inventors calculated the present call rate. Finally, the inventors performed quantitative quality control. Here, the absolute deviation of the mean expression values of each array from the overall mean was determined (R-project Vs 2.8.0) (Team RDC. R: A language and environment for statistical computing. R Foundation for Statistical Computing, 2006). In short, the mean expression value for each array was calculated. Next the mean of these mean expression values (overall mean) was taken and the deviation of each array mean from the overall mean was determined (analogous to probe outlier detection used by Affymetrix before expression value calculation) (Affymetrix. Statistical algorithms description document. 2002; http://www.affymetrix.com/support/technical/whitepapers/sadd_whitepaper.pdf). Arrays were only included in the study, if all three quality control methods confirmed sufficient quality, Two samples did not pass these quality controls (e.g. FIG. 6).

### Classification Algorithm

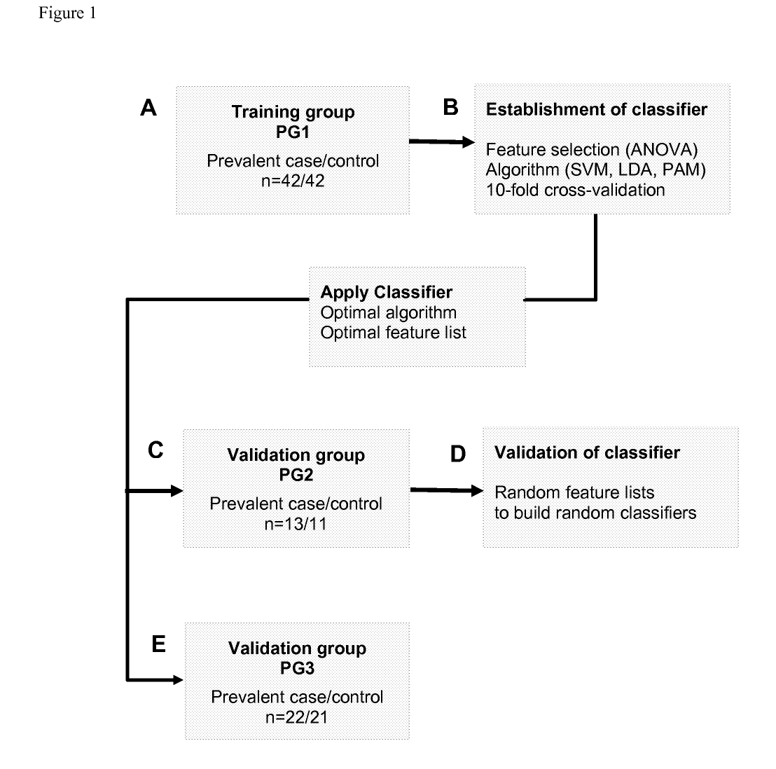

Expression values were independently quantile normalized. A classifier for lung cancer was built using the following machine learning algorithms: support vector machine (SVM), linear discrimination analysis (LDA), and prediction analysis for microarrays (PAM) using a 10-fold cross-validation design as described below. A schematic view of this approach is depicted in FIG. 1. Eighty-four samples were used in the training set (FIG. 1A). In the 10-fold cross-validation, the inventors randomly split the training group 10 times in a ratio 9:1. Differentially expressed transcripts between non-small-cell lung cancer, small-cell lung cancer and controls were identified using F-statistics (ANOVA) for each data set splitting in the larger data set split. Thirty six different feature lists were obtained as input for the classifier by sequentially increasing the cut-off value for the F-statistics (p=0.00001, p=0.00002, p=0.00003=0.08, p=0.09, p=0.1). The maximum feature size was restricted to 5 times the sample size to control for overfitting in this step (FIG. 1B) (Allison D B, Cui X, Page G P, Sabripour M. Microarray data analysis: from disarray to consolidation and consensus. Nat Rev Genet 2006; 7: 55-65). These selected features were used as input for each of the three machine learning algorithms (LDA, PAM, SVM). The optimal cut-off of the F-statistics and the optimal classification algorithm were selected according to the mean area under the receiver operator curve in this 10-fold cross-validation design in the training group (FIG. 1B). The inventors subsequently built a classifier using this cut-off value of the F-statistics and the selected algorithm in the whole prevalent training group (PG1). To further control for overfitting (Lee S. Mistakes in validating the accuracy of a prediction classifier in high-dimensional but small-sample microarray data. Stat Methods Med Res 2008; 17: 635-42), the classifier was validated in an independent group of matched cases and controls (PG2) (FIG. 1C). The area under the receiver operator curve was used to measure the quality of the classifier. Sensitivity and specificity were calculated at the maximum Youden-index (sensitivity+specificity−1) within the SVM probability range from 0.1-0.9. In addition, the inventors analyzed the single SVM probabilities for each case. To test the specificity of the classifier the whole analysis was repeated thousand times using random feature sets of equal size (FIG. 1D). A second validation group (PG3) was additionally used (FIG. 1E).

### Computational Data Analysis:

### Cross-Validation:

For 10-fold cross-validation the whole initial training group (PG1) was split 10 times in a ratio of 9:1 into an internal cross-validation training and validation group. Each sample was used only once for each internal validation group. As the number of samples is discrete, the inventors generated 6 internal validation sets with 8 samples and 4 validation sets with 9 samples. The calculation of the F-statistics was performed separately for each internal data set splitting. Based on the identified differentially expressed genes a classifier was built for each internal data set splitting and applied to the remaining internal validation group. For each internal validation group the given SVM scores of samples were used to build a receiver operator curve and calculate the area under this curve (AUC). After separate calculation of 10 AUCs the mean of these 10 AUCs was calculated. This mean AUC was used as read-out for the quality of the classifier. The settings of the best classifier as defined by the maximum mean AUC was used to then build a classifier on the whole training group and apply this classifier to an external independent validation group (PG2).

To avoid artificial optimization due to data set splitting into training (PG1) and independent validation group (PG2), the inventors performed the above described procedure of 10-fold cross-validation in 10 distinct random data-set splitting of a merged data-set from PG1 and PG2. For this random data-set splitting each sample was taken only once for the validation group. The whole test procedure described above was performed for each new data set splitting into test and validation group. For each of these data-set splittings into training and validation group the AUC of the classifier in the validation group was calculated. Finally, the mean and the standard deviation of these 10 AUCs were calculated.

A priori the optimal set of genes for the classifier is not known. The inventors used F-statistics to identify differentially expressed genes. This F-statistics was calculated separately for each single data-set in each cross-validation (n=100). In the next step, the inventors obtained 36 different lists of genes from each F-statistic by step-wise increase of the cut-off for the p-value of the F-statistic (p=0.00001, p=0.00002, p=0.00003 . . .-. . . p=0.08, p=0.09, p=0.1). Two rules were used to choose the optimal set of genes. (i) The optimal set of genes should lead to the maximum AUC. (ii) The number of genes involved in the classifier should be as low as possible to avoid overfitting.

To underline the specificity of the lung cancer specific transcripts extracted, the inventors performed a permutation analysis using 1000 randomly chosen feature lists of the same length as used for the classifiers.

### Algorithms for Classification:

The inventors used three different machine learning algorithms (support vector machine (SVM), linear discrimination analysis (LDA), and prediction analysis for microarrays (PAM)) for classification. All three machine learning algorithms were used as implemented in R. The following settings were used for these algorithms:

SVM: SVM is a well-established machine learning algorithm for distinction between two groups. Using the Kernel function it allows the identification of an optimal hypergeometric plane. scale=default, leading to an internal scaling of the x and y variable to 0 and unit variance; type=C-classification; kernel=linear; probability=true, allowing for probability predictions.

LDA: prior=default, no indication of prior probability of class membership was used leading to a probability equally to the class distribution in the training set; no additional argument was indicated.

PAMR: nfold=10, a 10-fold cross-validation was used; folds=default, a balanced random cross-validation was used; no further argument was added.

### Datamining:

To investigate gene ontology of transcripts used for the classifier, the inventors performed GeneTrail analysis for over- and underexpressed genes (Backes C, Keller A, Kuentzer J, et al. GeneTrail—advanced gene set enrichment analysis. Nucleic Acids Res 2007; 35: W186-92). To this end, the inventors analyzed the enrichment in genes in the classifier, compared to all genes present on the whole array. The inventors analyzed under-respectively over-expressed genes using the hypergeometric test with a minimum of 2 genes per category.

In addition, the inventors performed datamining by Gene Set Enrichment Analysis (GSEA) (Subramanian A, Tamayo P, Mootha V K, et al. Gene set enrichment analysis: a knowledge-based approach for interpreting genome-wide expression profiles, Proc Natl Acad Sci USA 2005; 102: 15545-50). As indicated, the inventors compared the respective list of genes obtained in the inventors' expression profiling experiment with datasets deposited in the Molecular Signatures Database (MSigDB). The power of the gene set analysis is derived from its focus on groups of genes that share common biological functions. In GSEA an overlap between predefined lists of genes and the newly identified genes can be identified using a running sum statistics that leads to attribution of a score. The significance of this score is tested using a permutation design, which is adapted for multiple testing (Subramanian A, Tamayo P, Mootha V K, et al. Gene set enrichment analysis: a knowledge-based approach for interpreting genome-wide expression profiles. Proc Natl Acad Sci USA 2005; 102: 15545-50). Groups of genes, called gene sets were deposited in the MSigDB database and ordered in different biological dimensions such as cancer modules, canonical pathways, miRNA targets, GO-terms etc. (http://www.broadinstitute.org/gsea/msigdb/index.jsp). In the analysis, the inventors focused on canonical pathways and cancer modules. The cancer modules integrated into the MSigDB are derived from a compendium of 1975 different published microarrays spanning several different tumor entities (Segal E, Friedman N, Koller D, Regev A. A module map showing conditional activity of expression modules in cancer. Nat Genet 2004; 36: 1090-8).] The gene sets used for the canonical pathway analysis were derived from several different pathway databases such as KEGG, Biocarta etc (http://www.broadinstitute.org/gsea/msigdb/collection_details.jsp#CP).

### Results

### Expression Profiling-Based Detection of Prevalent Lung Cancer

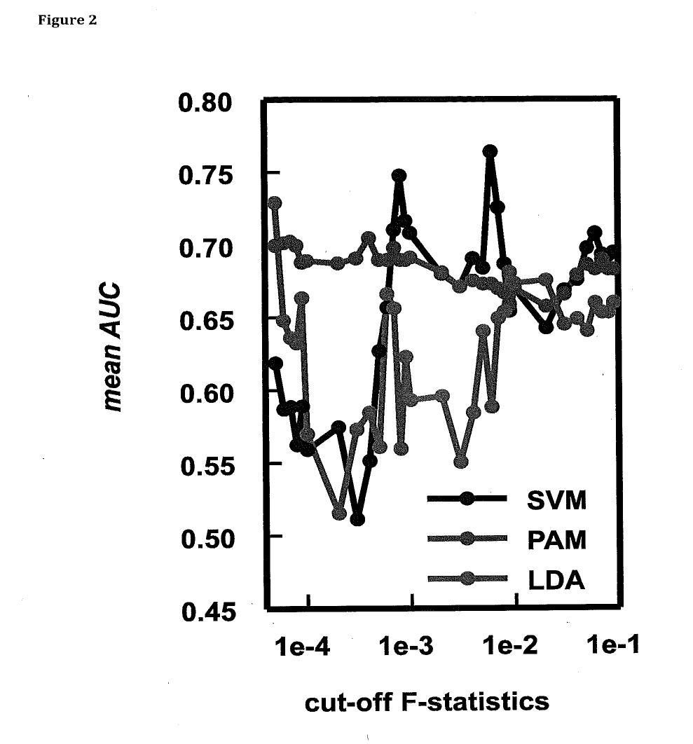

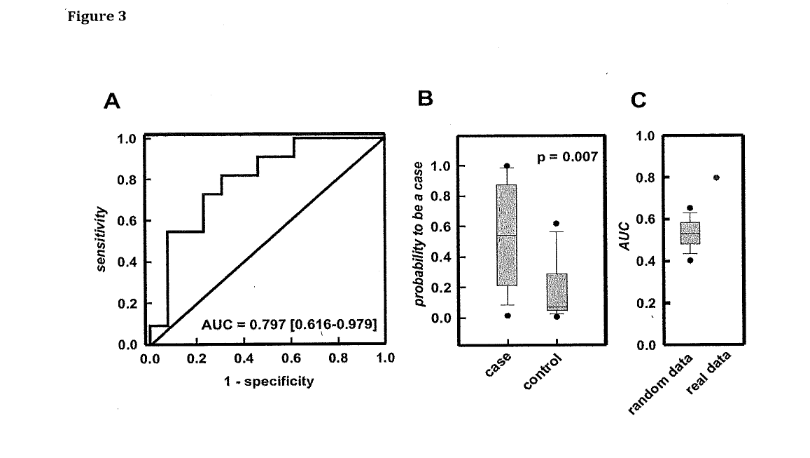

In the first case-control group of lung cancer patients (PG1) the highest accuracy for diagnosing prevalent lung cancer from blood-based transcription profiles was reached using a support vector machine (SVM-) based algorithm (FIG. 2). The highest mean AUC values in this 10-fold cross-validation were 0.747 (+/−0.206 standard deviations (std)) with a cut-off value for the F-statistic of 0.0008 and 0.763 (+/−0.189 std) with a cut-off for the F-statistic of 0.006, respectively. (FIG. 2). The inventors subsequently used a cut-off of 0.0008 to control for overfitting. Using this cut-off value, the inventors selected 161 transcripts as best performing feature set in the whole PG1 data set (Table 3) and used SVM to build a classifier. The inventors then used these transcripts and the same SVM model to classify samples from an independent validation case-control group (PG2). When using this classifier to build a receiver operator curve, the inventors calculated the AUC for the diagnostic test to be 0.797 [95% confidence interval (CI)=0.616-0.979] (FIG. 3A). In the PG2 validation cohort, the sensitivity for diagnosis of lung cancer was calculated to be 0.82 and the specificity 0.69 at the point of the maximum Youden index. Given the continuous nature of the SVM score additional use can be made from this score e.g. to increase specificity which might be useful depending on the potential application. E.g. using a cut-off of the SVM score of >0.9 leads to a specificity of 91% reducing the number of false positives by 27%.

In addition, the inventors observed a significant difference between the SVM scores of lung cancer cases and respective controls in the validation group (p=0.007, T test) (FIG. 3B). To underline the specificity of this test, the inventors used 1000 random lists each comprising 161 transcripts to build the classifier in PG1 and apply it to PG2. The mean AUC obtained by these random lists was 0.53 and not a single permutation (AUC range 0.31 to 0.78) reached the AUC of 0.797 of the lung cancer classifier (FIG. 3C). This translated into a p-value of less than 0.001 for the permutation test confirming the specificity of the lung cancer classifier.

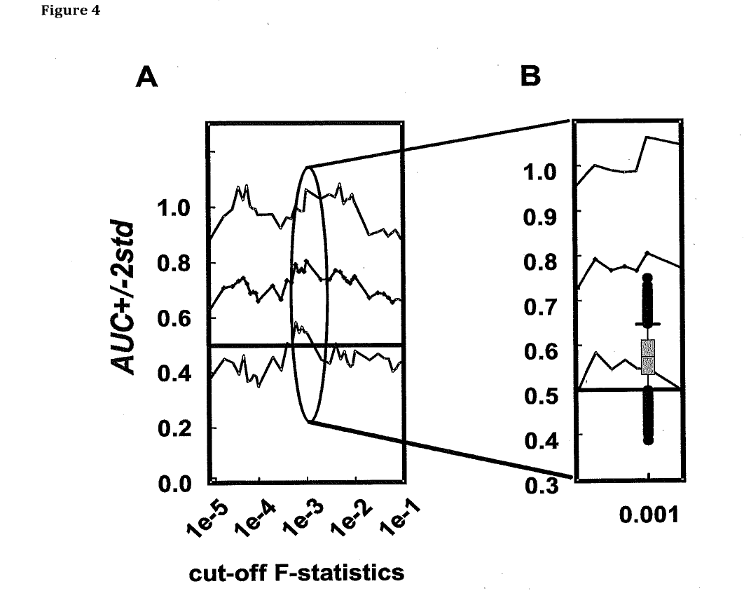

Next, the inventors excluded that the high AUC of the lung cancer classifier might be due to the elected splitting of the groups PG1 and PG2 into test and validation cohort. To this end, the inventors performed 10 random data set splittings of the merged PG1 and PG2 data sets and repeated the analysis for each data set splitting independently. For cut-off values of the F-statistics from 0.0006-0.001 the mean AUC of the 10 data set splittings was significantly above the expected random AUC of 0.5 (>2 standard deviations) (FIG. 4A), demonstrating that the results obtained were not due to specific splitting of the data set. The specificity of these findings is highlighted by the fact that none of the 1000 random feature lists of equal size led to an AUC as high as the mean AUC obtained by disease specific transcripts (FIG. 4B). To further underline the stability of the extracted feature list the differential expression of the extracted features was analyzed in each of the 10 random data set splittings in the merged PG1 and PG2 data set. 45% of all the transcripts of the initially extracted transcripts were differentially expressed in at least one random data set splitting at a p-value below 0.0008 in the F-statistics with 19.3% demonstrating a p-value below 0.0008 in all data set splittings (Table 4). Furthermore, 97% of the transcripts selected demonstrated a significant differential expression in all other dataset splitting, whereas only 7.6% of all random features were significantly different between the cases and controls at a p-value of below 0.05.

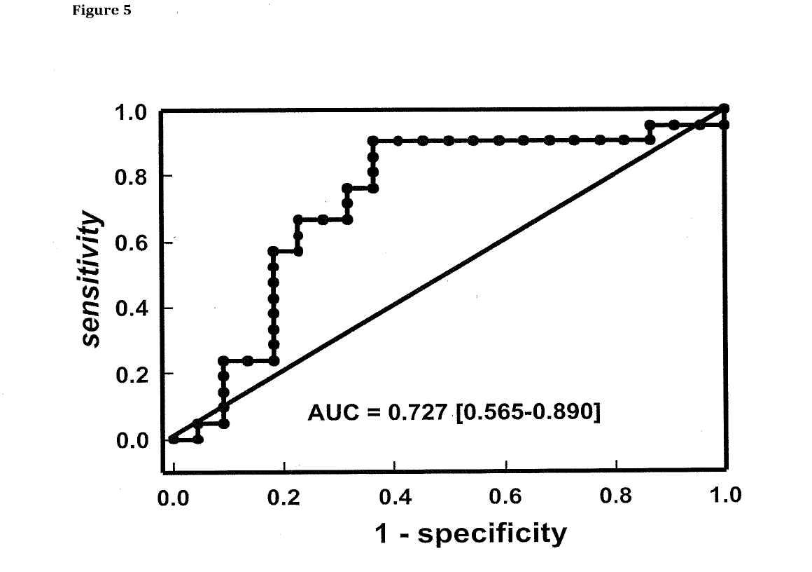

Additionally, the inventors tested the classifier built in PG1 in a third group of unmatched prevalent cases and controls (PG3). The AUC determined for this group was 0.727 [95% CI=0.565-0.890]. Thus, the performance of the classifier is independent of the presence of matched controls in the data set analyzed, further supporting the validity of these findings (FIG. 5).

In addition, the inventors generalized the results from the previous analysis by automation of the random re-division of samples into training group (PG1) and validation groups (PG2 and PG3). This automated process and evaluation for effective classifiers in the specific grouping was repeated 10.000 times. Genes/transcripts were ranked by the frequency of their appearance in these random groupings. The top 200 RNAs are listed in Table 3b.

Combinations of RNAs from Table 3 and combinations of RNAs from Tables 3 and 3b are differentiated by clinical utility: Table 3 only combinations are selected, trained and validated on different sets with defined clinical properties, while Table 3b extends the gene/transcript selection with a generalization of the results across all samples. A combination of genes/transcripts from Tables 3 and 3b (or of Table 3b alone) of technically appropriate size is an optimal candidate for validation in a new set of samples or a prospective study.

Therefore, one aspect of the invention pertains to a method for the detection of lung cancer in a human subject based on RNA from a blood sample obtained from said subject, comprising: measuring the abundance of at least 4 RNAs in the sample, that are chosen from the RNAs listed in table 3b, and concluding based on the measured abundance whether the subject has lung cancer. Another aspect of the invention pertains to a microarray, comprising a solid support and a set of oligonucleotide probes, the set containing from 5 to about 3,000 probes, and including at least 4 probes for detecting an RNA selected from Table 3b. Another aspect of the invention pertains to the use of a microarray for detection of lung cancer in a human subject based on RNA from a blood sample, comprising measuring the abundance of at least 4 RNAs listed in table 3b, wherein the microarray comprises at least 4 probes for measuring the abundance of each of at least 4 RNAs. Another aspect of the invention pertains to a kit for the detection of lung cancer in a human subject based on RNA obtained from a blood sample, comprising means for measuring the abundance of at least 4 RNAs that are chosen from the RNAs listed in table 3b, preferably comprising means for exclusively measuring the abundance of RNAs that are chosen from table 3b. Another aspect of the invention pertains to the use of a kit as mentioned above for the detection of lung cancer in a human subject based on RNA from a blood sample, comprising means for measuring the abundance of at least 4 RNAs that are chosen from the RNAs listed in table 3b, comprising measuring the abundance of at least 4 RNAs in a blood sample from a human subject, wherein the at least 4 RNAs are chosen from the RNAs listed in table 3b, and concluding based on the measured abundance whether the subject has lung cancer. Another aspect of the invention pertains to a method for preparing an RNA expression profile that is indicative of the presence or absence of lung cancer in a subject, comprising isolating RNA from a blood sample obtained from the subject, and determining the abundance of from 4 to about 3000 RNAs, including at least 4 RNAs selected from Table 3b.

### Mining of Expression Profiles

To analyze the biological significance of the differentially expressed transcripts different strategies were used. First, the inventors used GeneTrail (Backes C, Keller A, Kuentzer J, et al. GeneTrail—advanced gene set enrichment analysis. Nucleic Acids Res 2007; 35: W186-92) to analyze an enrichment in GO-terms of the genes specific for lung cancer in the inventors' study (n=161) (Table 3). The inventors observed 10 GO categories demonstrating a significant (p-value FDR corrected <0.05) enrichment of genes in this classifier (GO:0002634: regulation of germinal center formation; GO:0043231: intracellular membrane-bounded organelle, GO:0000166: nucleotide binding, GO:0043227: membrane-bounded organelle, GO:0042100: B cell proliferation, GO:0002377: immunoglobulin production, GO:0046580: negative regulation of Ras protein signal transduction, GO:0002467, GO:0051058: germinal center formation, GO:0017076: purine nucleotide binding). Six of these GO categories are part of the biological subtree comprising 4 categories of genes associated with the immune system. (GO:0002634, GO:0042100, GO:0002377, GO:0051058) These data indicate an impact of immune cells to the genes involved in the classifier.

Second, the inventors analyzed the 1000 transcripts most significantly changed within the dataset between NSCLC, SCLC and controls (Table 5). The inventors computed overlaps between these annotated transcripts and the gene set collection deposited in the Molecular Signature Database focusing on the canonical pathways. The pathway gene sets are curated sets of genes from several pathway databases (http://www.broadinstitute.org/gsea/msigdb/collection_details.jsp#CP). These pathways point to potential biological functions the group of genes is involved in. Of the 1000 transcripts differentially expressed in the inventors' study, 776 were present in the Molecular Signature Database. When calculating the overlap between the inventors' lung cancer specific gene set and the canonical pathways gene set, the inventors observed 11 canonical pathway gene sets with significant (corrected p-value<0.05, p<2.9*10⁻⁵ uncorrected) overlap 4 of which can be partly attributed to interaction of immune cells (HSA04060 cytokine cytokine receptor interaction (uncorrected p-value=5.11×10⁻⁸), HSA04010 MAPK signaling pathway (uncorrected p-value=6×10⁻⁷), HSA01430 cell communication (uncorrected p-value=7.8 10⁻⁷), HSA04510 focal adhesion (uncorrected p-value=2.9*10⁻⁵). These data further underline an enrichment of immune associated genes in the lung cancer specific expression profile.

Third, the inventors performed a gene set enrichment analysis (Subramanian A, Tamayo P, Mootha V K, et al. Gene set enrichment analysis: a knowledge-based approach for interpreting genome-wide expression profiles. Proc Natl Acad Sci USA 2005; 102: 15545-50; Segal E, Friedman N, Koller D, Regev A. A module map showing conditional activity of expression modules in cancer. Nat Genet 2004; 36: 1090-8) with a focus on cancer modules which comprise groups of genes participating in biological processes related to cancer. Initially, the power of such modules has been demonstrated exemplarily for single genes such as cyclin D1 or PGC-1alpha (Lamb J, Ramaswamy S, Ford H L, et al. A mechanism of cyclin D1 action encoded in the patterns of gene expression in human cancer. Cell 2003; 114: 323-34; Mootha V K, Lindgren C M, Eriksson K F, et al. PGC-1alpha-responsive genes involved in oxidative phosphorylation are coordinately downregulated in human diabetes. Nat Genet 2003; 34: 267-73) and a more comprehensive view on such modules has been introduced recently (Segal E, Friedman N, Koller D, Regev A. A module map showing conditional activity of expression modules in cancer. Nat Genet 2004; 36: 1090-8). This comprehensive collection of modules allows the identification of similarities across different tumor entities such as the common ability of a tumor to metastasize to the bone e.g. in subsets of breast, lung and prostate cancer (Segal E, Friedman N, Koller D, Regev A. A module map showing conditional activity of expression modules in cancer. Nat Genet 2004; 36: 1090-8). Overall 456 such modules are described in the database spanning several biological processes such as metabolism, transcription, cell cycle and others. For this analysis, the inventors explored only those genies, which were identified to be discriminative between cases and controls in the inventors' data set independent of the data set splitting (n=31) (Table 4). Within this set of 31 genes the inventors observed a significant enrichment of the genes related to modules 543, 552, 168, 222, 421. Interestingly, these specific modules are also mainly observed in lung cancer samples in the original sample collection of 1975 samples. Although the lung cancer samples account only for 13% of the deposited samples the above mentioned modules are preferentially present in these lung cancer samples (average 8.6 samples). In contrast, in non-lung cancer samples accounting for 87% of the deposited samples these modules were rarely observed (average 3.6) (Segal E, Friedman N, Koller D, Regev A. A module map showing conditional activity of expression modules in cancer. Nat Genet 2004; 36: 1090-8). This indicates that genes differentially expressed in peripheral blood between lung cancer cases and controls in the inventors' study are part of biologically cooperating genes that are also differentially expressed in primary lung cancer but not in other cancer entities. Many of the genes within these cancer modules have phosphotransferase activity (GNE, GALT, SRPK1, PFTK1, STK17B, PIP4K2B) and are involved in cell signaling. To underline this specificity for lung cancer of the genes extracted in the analysis, the inventors further calculated the overlap between the inventors' extracted gene set (n=161) and the genes differentially expressed in blood of patients with renal cell cancer (Twine N C, Stover J A, Marshall B, et al. Disease-associated expression profiles in peripheral blood mononuclear cells from patients with advanced renal cell carcinoma. Cancer Res 2003; 63: 6069-75). Only CD9 was present in both gene sets. Similarly no overlap was observed between the inventors' gene set that was used for classification of samples (n=161) and blood based expression profiles for melanoma (Critchley-Thorne R J, Yan N, Nacu S, Weber J, Holmes S P, Lee P P. Down-regulation of the interferon signaling pathway in T lymphocytes from patients with metastatic melanoma. PLoS Med 2007; 4: e176), breast (Sharma P, Sahni N S, Tibshirani R, et al. Early detection of breast cancer based on gene-expression patterns in peripheral blood cells. Breast Cancer Res 2005; 7: R634-44) and bladder (Osman I, Bajorin D F, Sun T T, et al. Novel blood biomarkers of human urinary bladder cancer. Clin Cancer Res 2006; 12: 3374-80). In summary, these data point to a lung cancer specific gene set present in the inventors' classifier.

Using RNA-stabilized whole blood from smokers in three independent cohorts of lung cancer patients and controls, the inventors present a gene expression based classifier that can be used to discriminate between lung cancer cases and controls. Applying a classical 10-fold cross-validation approach to a first cohort of patients (PG1), the inventors determined a lung cancer specific classifier. This classifier was successfully applied to two independent cohorts (PG2 and PG3). Extensive permutation analysis as well as random feature set controls and random data set splittings further showed the specificity of the lung cancer classifier.

Overall, the inventors' data demonstrate the feasibility and utility of a diagnostic test for lung cancer based on RNA-stabilized whole blood in smoking patients, in particular with a high degree of comorbidity.

1. A method for the detection of lung cancer in a human subject based on RNA from a blood sample obtained from said subject, comprising:
Measuring the abundance of at least 4 RNAs in the sample, that are chosen from the RNAs listed in table 3 or in table 3b, and
Concluding based on the measured abundance whether the subject has lung cancer.

2. The method of claim 1, wherein the abundance of at least 9 RNAs, of at least 10 RNAs, of at least 13 RNAs, of at least 29 RNAs that are chosen from the RNAs listed in table 3 or in table 3b is measured.

3. The method of claim 1, wherein the abundance of at least the 161 RNAs of table 3 is measured.

4. The method of claim 1, wherein the measuring of RNA abundance is performed using a microarray, a real-time polymerase chain reaction or sequencing.

5. The method of claim 1, wherein the decision whether the subject has lung cancer comprises the step of training a classification algorithm on a training set of cases and controls, and applying it to measured RNA abundance.

6. The method of claim 1, wherein the classification method is a random forest method, a support vector machine (SVM), or a K-nearest neighbor method (K-NN), such as a 3-nearest neighbor method (3-NN).

7. The method of claim 1, wherein the RNA is mRNA, cDNA, micro RNA, small nuclear RNA, unspliced RNA, or its fragments.

8. The method of claim 1, wherein the abundance of at least 1 RNA in the sample is measured that is chosen from the RNAs listed in table 3b together with measuring the abundance of at least 4 RNAs in the sample, that are chosen from the RNAs listed in table 3.

9. Use of a method of claim 1 for detection of lung cancer in a human subject based on RNA from a blood sample.

10. A microarray, comprising a solid support and a set of oligonucleotide probes, the set containing from 5 to about 3,000 probes, and including at least 4 probes for detecting an RNA selected from table 3, preferably also including at least one probe for detecting an RNA selected from table 3b, or including at least 4 probes for detecting an RNA selected from table 3b.

11. Use of a microarray for detection of lung cancer in a human subject based on RNA from a blood sample, comprising measuring the abundance of at least 4 RNAs listed in table 3, wherein the microarray comprises at least 4 probes for measuring the abundance of each of at least 4 RNAs, preferably also comprising measuring the abundance of at least 1 RNA listed in table 3b, wherein the microarray preferably also comprises at least one probe for measuring the abundance of the at least 1 RNA of table 3b, or comprising measuring the abundance of at least 4 RNAs listed in table 3b, wherein the microarray comprises at least 4 probes for measuring the abundance of each of at least 4 RNAs.

12. A kit for the detection of lung cancer in a human subject based on RNA obtained from a blood sample, comprising means for measuring the abundance of at least 4 RNAs that are chosen from the RNAs listed in table 3 or in table 3b, preferably comprising means for exclusively measuring the abundance of RNAs that are chosen from table 3 or from table 3b, respectively.

13. The kit of claim 12, comprising means for measuring the abundance of at least 1 RNA that is chosen from the RNAs listed in table 3b together with means for measuring the abundance of at least 4 RNAs that are chosen from the RNAs listed in table 3, preferably comprising means for exclusively measuring the abundance of RNAs that are chosen from table 3 and of the at least one RNA that is chosen from table 3b.

14. Use of a kit of claim 12 for the detection of lung cancer in a human subject based on RNA from a blood sample, comprising means for measuring the abundance of at least 4 RNAs that are chosen from the RNAs listed in table 3 or in table 3b, comprising
Measuring the abundance of at least 4 RNAs in a blood sample from a human subject, wherein the at least 4 RNAs are chosen from the RNAs listed in table 3 or in table 3b, and
Concluding based on the measured abundance whether the subject has lung cancer.

15. Use of a kit of claim 13, comprising
Measuring the abundance of at least 4 RNAs in a blood sample from a human subject, wherein the at least 4 RNAs are chosen from the RNAs listed in table 3,
Measuring the abundance of at least 1 RNA in the blood sample, wherein the at least 1 RNA is chosen from the RNAs listed in table 3b, and
Concluding based on the measured abundance whether the subject has lung cancer.

16. A method for preparing an RNA expression profile that is indicative of the presence or absence of lung cancer in a subject, comprising:
Isolating RNA from a blood sample obtained from the subject, and
Determining the abundance of from 4 to about 3000 RNAs, including at least 4 RNAs selected from table 3, and preferably including at least 1 RNA selected from table 3b, or including at least 4 RNAs selected from table 3b.
